(19) United States
(12) Patent Application Publication
(10) Pub. No.: US 2008/0171062 A1
(43) Pub. Date: Jul. 17, 2008

(54) Recombinant HBsAg virus-like particles containing polyepitopes of interest, their production and use

(76) Inventors: Monica Sala-Schaeffer, Paris (FR); Marie Michel, Raely Les Hateaux (FR); Simon Wain-Hobson, Montigny-le-Bretonneux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/889,217

(22) Filed: Aug. 9, 2007

Related U.S. Application Data
(60) Provisional application No. 60/837,909, filed on Aug. 16, 2006.

(51) Int. Cl. A61K 39/29; C12N 15/63; C12N 1/19; C12N 1/15; C12N 15/51; C12N 1/21; C12N 5/10; C12P 21/00; C07K 14/02

(52) U.S. Cl. 424/204.1; 435/320.1; 435/254.2; 435/254.11; 435/419; 435/325; 435/69.1; 530/350; 536/23.72; 435/252.3

(57) ABSTRACT

The hepatitis B surface antigen (HBsAg) can assemble into sub-virion virus like particles (VLPs). By fusing immunogenic peptides to the amino-terminus of HBsAg, several bivalent vaccines have been developed. In one example, an optimized HIV-1 polyepitope-HBsAg recombinant protein assembled into VLPs and was efficiently secreted. DNA immunization in mice resulted in the induction of humoral neutralising response against the carrier and enhanced levels of HIV-1 specific CD8⁺ T lymphocytes activation.

### CROSS-REFERENCE TO RELATED APPLICATIONS

This application is based on and claims the benefit of U.S. Provisional Application No. 60/837,909, filed Aug. 16, 2006 (Attorney Docket No. 03495.6115) The entire disclosure of this application is relied upon and incorporated by reference herein.

### FIELD OF THE INVENTION

This invention relates to recombinant hepatitis B surface antigen (HBsAg) virus-like particles (VLPs) and to their production and to their use in therapeutic applications. The recombinant HBsAg virus-like particles contain heterologous polyepitopes fused to the middle (M) envelope protein. The invention also relates to heterologous polyepitopes and to polynucleotide encoding the heterologous polyepitopes. The HBsAg virus-like particles are particularly useful in immunogenic compositions and as vaccines.

### BACKGROUND OF THE INVENTION

Many viral structural proteins have the intrinsic ability to assemble into virus-like particles (VLPs) independently of nucleic acids. VLPs can elicit potent anti-viral humoral and cellular immune responses directed against viruses they derive from (10, 24, 36, 37). They are efficiently taken up, rapidly internalised, and processed by antigen presenting cells (APCs) of myeloid origin, leading to MHC class I-associated antigen cross-presentation (1, 17, 33-35, 38). Indeed, MHC class I cross-presentation of VLP epitopes by APCs can be exploited to induce anti-viral CD8+ cytotoxic T lymphocyte (CTL) responses. VLPs are powerful antigen delivery systems, the most developed examples being the hepatitis B surface antigen (HBsAg) (Li H Z, Gang H Y, Sun Q M, Liu X, Ma Y B, Sun M S, et al. Production in Pichia pastoris and characterization of genetic engineered chimeric HBV/HEV virus-like particles. Chin Med Sci J 2004; 19(2):78-83. Pumpens P, Razanskas R, Pushko P, Renhof R, Gusars I, Skrastina D, et al. Evaluation of HBs, HBc, and frCP virus-like particles for expression of human papillomavirus 16 E7 oncoprotein epitopes. Intervirology 2002; 45(1):24-32.) Yang H J, Chen M, Cheng T, He S Z, Li S W, Guan B Q, et al. Expression and immunoactivity of chimeric particulate antigens of receptor binding site-core antigen of hepatitis B virus. World J Gastroenterol 2005; 11(4):492-97), the yeast Ty retrotransposon structural protein “a” (Tya) (Roth J F. The yeast Ty virus-like particles. Yeast 2000; 16(9):785-95), the VP2 capsid protein of porcine parvovirus (PPV) (Sedlik C, Saron M, Sarraseca J, Casal I, Leclerc C. Recombinant parvovirus-like particles as an antigen carrier: a novel nonreplicative exogenous antigen to elicit protective antiviral cytotoxic T cells. Proc Natl Acad Sci USA 1997; 94(14):7503-8), and the papillomavirus capsid L1 protein (Buck C B, Pastrana D V, Lowy D R, Schiller J T. Generation of HPV pseudovirions using transfection and their use in neutralization assays. Methods Mol Med 2005; 119:445-62). The generation of recombinant VLPs bearing relevant antigens opens up the way to the development of bivalent vaccine candidates (19, 21, 30).

The native forms of hepatitis B surface antigen (HBSAg) are the three envelope proteins of hepatitis B virus (HBV), known as the large (L), the middle (M) and the small (S, otherwise known as the major) envelope proteins. The HBV envelope gene encoding the HBV envelope proteins carrying the surface antigen determinants has a single open reading frame (orf) containing three in frame ATG start codons that divide the gene into three coding regions known as preS1, preS2 and S (proceeding in a 5′ to 3′ direction). The three different-sized envelope proteins are encoded by distinct regions of the orf as a result of two different mRNA transcripts: L by preS1+preS2+S regions, M by preS2+S regions and S by S region. A bicistronic mRNA encodes both M and S envelope proteins, with preS2 translation initiation codon less efficient than the S region one (14).

HBsAg carries all the information necessary for membrane translocation, particle assembly, and secretion from mammalian cells (5). Substitutions within HBsAg that impair VLPs assembly are generally characterized by HBsAg accumulation in the endoplasmic reticulum (ER) and Golgi apparatus (8).

By fusing foreign DNA to the HBV envelope gene, HBsAg has been used as carrier for a wide panel of antigens (12, 19, 21, 27, 30). In a notable example, a series of 13 HIV-1 epitopes restricted by the HLA-A*0201 class I allele, which is present at ˜15-30% of Black, Caucasian, and Oriental populations, was incorporated into the preS2 region as a polyepitope (polHIV-1) fused to HBsAg. Although the study reported the induction of HIV-1 specific CTL responses by DNA vaccination (12) of humanised HLA-A*0201 transgenic mice (11), it was not shown whether the recombinant HBsAg actually formed VLPs.

The polHIV-1 polyepitope was characterised by a number of traits that might prevent VLPs assembly and impinge on immunogenicity. Firstly, the epitopes were fused directly head-to-tail, which could possibly induce silencing by immunodominant-epitopes (40). Secondly, the presence of basic, amide, or small residues as first residue carboxy-terminal (C1-) to an epitope, which has been demonstrated to enhance immunogenicity, was not taken into account (20). Finally, the polyepitope was remarkably hydrophobic on a par with membrane spanning peptides. There were five cysteine and four methionine codons, one of which must be considered as the equivalent of an efficient translation initiation codon. This latter feature contrasts with the characteristics of all mammalian preS1 and preS2 coding regions, i.e., a generally hydrophilic profile and the complete absence of cysteine codons and methionine codons apart from those used to initiate preS1 and preS2 translation. By impairing VLPs assembly, such features may impact on efficient antigen cross-presentation and immune response against the HBsAg carrier.

Thus, there exists a need in the art for recombinant polyepitopes such as polyepitopes from pathogens as HIV, suitable for, among other things, insertion into the preS2 region of the M envelope protein compatible with VLPs formation. Recombinant VLPs secretion should result in the induction of robust neutralising anti-HBsAg humoral and cellular immune responses and the induction of polyepitope-specific CD4⁺ and/or CD8⁺T lymphocytes so that the VLPs can be employed in therapeutic applications.

### SUMMARY OF THE INVENTION

A previous HLA.A2.1-restricted HIV-1 polyepitope was constructed with the aim of triggering an antiviral cellular immune response (12). It has been discovered by inventors of the present patent application that fused to the M envelope protein, this polyepitope impairs the secretion of virus-like particles (VLPs). This invention involves the design of polyepitopes, such as the polHIV-1.opt polyepitope of the invention, in which secretion of HBsAg VLPs containing polyepitopes is rescued. In a preferred embodiment of the invention, HLA.A2.1- and HLA.B7-restricted HIV-1 polyepitopes have been designed, and positively tested by the present inventors for preservation of recombinant HBsAg VLPs secretion.

Thus, in one aspect, this invention concerns: i) the optimization parameters employed in the design of MHC class I-restricted polyepitopes to be produced as fusion protein at the surface of VLP; ii) the constructions obtained assembling the nucleic acids encoding new polypepitopes to expression vectors for optimal expression of recombinant VLPs; and iii) optimized polyepitopes and polynucleotides encoding them.

In particular, this invention aids in fulfilling the needs in the art by providing an expression vector for the production of virus-like particles comprising fusion proteins and S proteins of hepatitis B virus (HBV). The proteins are encoded by the preS2+S regions and S region of the HBV genome, respectively. The expression vector comprises a polynucleotide that encodes a polypeptide comprising a heterologous polyepitopic sequence of interest, wherein epitopes in the polyepitopic sequence are in head to tail position. The polynucleotide sequence is positioned in the preS2 region downstream of the preS2 ATG codon. The polynucleotide sequence is free of codons for cysteine and contains as few codon for methionine as possible. Polynucleotides encoding tetra-amino acid spacers between the head to tail epitopes in the polyepitopic sequence each comprise, for example, an arginine (R) residue placed in the epitope C₁-position directly linked to a sequence of three different amino acids independently selected from alanine (A), threonine (T), lysine (K), and aspartic acid (D). The preS2 translation initiation codon and S translation initiation codon are preserved so that S protein and the fusion protein comprised of M protein and the polypeptide comprising the polyepitopic sequence are translated. The S proteins and the fusion proteins assemble into virus-like particles after expression of the vector in a host cell. The polyepitopic sequence of interest can be from a pathogen, such as human immunodeficiency virus. In a preferred embodiment, the polynucleotide sequence is free of methionine codons. In another preferred embodiment, the polynucleotide sequence encodes polHIV-1.opt.

This invention also provides a host cell comprising a vector of the invention.

In addition, this invention provides a method of producing virus-like particles. The method comprises providing a host cell of the invention, and expressing the fusion protein and the S protein under conditions in which the proteins assemble into virus-like particles, which are released from the host cell into extracellular space.

Further, this invention provides virus-like particles comprising fusion proteins and S proteins of hepatitis B virus, wherein the proteins are encoded by modified-preS2+S regions and S region, respectively, of the HBV genome. A polypeptide is fused in-frame in the M protein downstream of the preS2 translation initiation methionine residue. The polypeptide is free of cysteine residues and contains 0 or 1 methionine residues. The polypeptide comprises a polyepitopic sequence of interest, wherein epitopes in the polyepitopic sequence are in head to tail position. Tetra-amino acid spacers between the head to tail epitopes in the polypeptide sequence each comprise, for example, an arginine (R) residue placed in the epitope C₁-position followed by three different amino acids independently selected from alanine (A), threonine (T), lysine (K), and aspartic acid (D). The S proteins and the fusion proteins are assembled into the virus-like particles.

A composition of the invention comprises the virus-like particles and a pharmaceutically acceptable carrier therefor.

This invention further provides a method for optimizing the immunogenicity of a polyepitopic sequence of interest for incorporation in a virus-like particle. The method comprises providing a polynucleotide sequence encoding a polyepitopic sequence of interest, wherein the polyepitopic sequence is comprised of epitopes in head-to-tail position. Codons for cysteine and the codons for methionine are removed from the polynucleotide sequence if the epitopes contain cysteine and methionine. Polynucleotides encoding tetra-amino acid spacers are provided between the epitopes in the polyepitopic sequence. Each spacer comprises, for example, an arginine residue placed in the epitope C₁-position directly linked to a sequence of three different amino acids independently selected from alanine, threonine, lysine, and aspartic acid. In a preferred embodiment, the method further comprises optimizing codon usage in the polyepitopic sequence based on preferred codon usage patterns in the human genome. This invention also provides a polynucleotide sequence obtained according to the method, and an expression vector comprising the polynucleotide sequence.

In addition, this invention provides a polyepitopic sequence encoded by the polynucleotide, and virus-like particles comprising the polyepitopic sequence. The virus-like particles can comprise, as a carrier for the polyepitopic sequence, a VLP chosen, for example, from HBsAg, HBc, frCP, HBV/HEV chimeras, yeast Ty, HPV, HCV, and parvovirus.

A fusion protein according to the invention comprises the polyepitopic sequence positioned within the preS2 region of an M protein of HBV. A preferred polyepitopic amino acid molecule is selected from polHIV-1.opt, pol1A2, pol2A2, pol1B7, and pol2B7.

Also, this invention provides an expression vector for the production of virus-like particles comprising fusion proteins and S proteins of hepatitis B virus (HBV). The proteins are encoded by the preS2+S regions and S region of the HBV genome, respectively. The expression vector comprises a polynucleotide sequence that encodes a polypeptide comprising a polyepitopic sequence. Epitopes in the polyepitopic sequence are in head to tail position. The polynucleotide sequence is positioned in the preS2 region downstream of the preS2 ATG codon, and the polynucleotide sequence is free of codons for cysteine and contains 0 or 1 codon for methionine apart from a methionine codon necessary to initiate preS2 translation. Polynucleotides encoding tetra-amino acid spacers between the head to tail epitopes in the polyepitopic sequence each comprises an amino acid residue placed in the epitope C₁-position directly linked to a sequence of three different amino acid residues. The amino acid residues are independently selected from alanine (A), threonine (T), lysine (K), aspartic acid (D), serine (S), glutamine (Q), asparagine (N), and histidine (H). Translation from preS2 and S ATG codons is preserved so that hepatitis B S protein and a fusion protein comprised of M protein and the polypeptide comprising the polyepitopic sequence are expressed, such that the HBsAg proteins and the fusion protein assemble into virus-like particles after expression of the vector in a host cell.

In another embodiment, virus-like particles comprise fusion protein and HBsAg proteins of hepatitis B virus, wherein the proteins are encoded by preS2+S region and the S region, respectively, of the HBV genome. A polypeptide is fused in-frame in the M protein downstream of the preS2 initiation methionine residue, wherein the polypeptide is free of cysteine residues and contains 0 or 1 methionine residues apart from methionine at the initiation site of preS2 translation, and wherein the polypeptide comprises a polyepitopic sequence of interest. Epitopes in the polyepitopic sequence are in head to tail position. Tetra-amino acid spacers between the head to tail epitopes in the polypeptide sequence each comprises an amino acid residue placed in the epitope C₁-position directly linked to a sequence of three different amino acid residues. The amino acid residues are independently selected from alanine (A), threonine (T), lysine (K), aspartic acid (D), serine (S), glutamine (Q), asparagine (N), and histidine (H). The HBsAg proteins and the fusion proteins are assembled into the virus-like particles.

Virus-like particles comprising the polyepitopic sequence are also provided, as is a fusion protein comprising the polyepitopic sequence positioned within the preS2 region of an M protein of HBV. A preferred polyepitopic amino acid molecule is selected from polHIV-1.opt, pol1A2, pol2A2, pol1B7, and pol2B7.

Also provided is a bacteria carrying the recombinant vector ppolHIV-1.opt (CNCM I-3547), pGA1xFlagMpol.opt (CNCM I-3544), pGA3xFlagMpol.opt (CNCM I-3546), pGA1xFlagM.pol1A2 (CNCM I-3579), pGA1xFlagM.pol2A2 (CNCM I-3580), pGA1xFlagM.pol1B7 CNCM (I-3581), or pGA1xFlagM.pol2B7 (CNCM I-3582).

In summary, the recombinant expression vector comprises a polynucleotide that encodes a polyepitope, i.e., a polypeptide comprising a polyepitopic sequence of interest. Epitopes in the polyepitopic sequence are in head to tail position. The polynucleotide is positioned in the preS2 region downstream of the preS2 ATG start codon, and the polynucleotide is free of codons for cysteine and contains as few codon for methionine as possible, insofar as they do no disturb the translation efficiency of the preS2 and S ATG start codons, the best being zero. Translation from preS2 and S ATG start codons is preserved so that the S envelope (HBsAg) protein and a fusion protein comprised of M protein and the fused in frame polypeptide comprising the heterologous polyepitopic sequence are produced. The HBsAg proteins and the fusion proteins assemble into virus-like particles after expression of the vector in an eukaryotic host cell.

The method of producing the virus-like particles of the invention comprises providing an eukaryotic host cell comprising a vector of the invention, and expressing the fusion protein and the S envelope (HBsAg) protein under conditions in which the proteins assemble into virus-like particles, which are released from the host cell into the extracellular space.

The virus-like particles comprising fusion proteins and S envelope (HBsAg) proteins, which are encoded by preS2+S regions and the S region, respectively, of the HBV envelope gene. A polypeptide is fused in-frame within the preS2 region of the M envelope protein. The polypeptide is free of cysteine residues and contains as few methionine residues as possible, insofar as they do no disturb the translation efficiency of the preS2 and S ATG start codons

The method of the invention optimizes the sequence of a polyepitope of interest, for example, a pathogen or a tumor polyepitope, for production in a virus-like particle.

The optimized polyepitopic sequences and polynucleotides encoding the optimized polyepitopic sequences, as well as fusion proteins containing the optimized polyepitopic sequences, are useful for the production of virus-like particles.

### BRIEF DESCRIPTION OF THE DRAWINGS

This invention will be described with reference to the drawings in which:

FIG. 1 relates to the two polyepitopes: polHIV-1 and polHIV-1.opt. (A) Schematic representation of recombinant HBsAg proteins: pre-S2: portion of the HBV pre-S2 protein conserved in the pCMV-B10 construct (12); HIV-1 polyepitope:amino acid sequences detailed in (B) and (C); V3 loop: envelope V3 loop of the MN HIV-1 isolate; HBsAg: hepatitis B virus surface antigen (otherwise identified herein as S envelope protein). The two ATG codons indicate the translation initiation methionines of fusion and HBsAg proteins, respectively. (B) Amino acid sequence of the polHIV-1 polyepitope. (C) Amino acid sequence of the polHIV-1.opt polyepitope. Spacers are underlined. From (D) the ppolHIV-1 plasmid, (E) the ppolHIV-1.opt plasmid, and (F) the HBV ayw isolate (accession number U95551): hydropathy profiles of the amino acid sequences from the preS2 ATG start codon to the HBsAg stop codon. Positive values correspond to hydrophobicity and negative to hydrophilicity.

FIG. 2 shows rescue of the VLPs secretion by the optimized polHIV-1.opt polyepitope. Mean values of samples in triplicate are given. (A) Detection of HBsAg antigenic units in VLPs by Monolisa Kit. Cut-off value was 0.1 ng/ml. (B) Anti-V3 loop ELISA analysis. Data are given as relative optical density values multiplied by 10³. Cut-off value was 15, determined as OD values corresponding to wells with the medium alone. (C) Detection of HBsAg antigenic units in VLPs by Monolisa Kit. 1:1 and 3:1 ratios correspond to the relative molar proportions of ppolHIV-1.opt and pCMV-S2.S plasmids in 2 μg of total DNA used for cotransfection. HBsAg ng/ml values are in log₁₀ scale. Cut-off value was 0.1 ng/ml.

FIG. 3 is a confocal immunofluorescence analysis obtained from SW480 cells transiently transfected by (A) ppolHIV-1, (B) ppolHIV-1.opt or (C) pCMV-basic plasmids. Each image corresponds to a plane projection of 16-20 focal plans. In green: Golgi staining; in red HBsAg staining.

FIG. 4 shows humoral immune responses in mice and INF-γ secretion in vitro assays. (A and B) Anti-HBsAg conformational IgGs ELISA assays on sera from (A) HHD transgenic mice (black spot) and (B) HLA-A*0201/HLA-DR1 double transgenic mice (grey diamond). Horizontal continuous lines correspond to cut-off values which result from mean values obtained from HHD and HLA-A*0201/HLA-DR1 naive mice, respectively. Positive values are boxed, and mean values of positive data are given as horizontal lines in the boxes. (C and D) INF-γ secretion is estimated as the percentage of INF-γ secreting (C) CD4+ T cells (values are in log₁₀ scale), and (D) CD8⁺ T cells on total lymphocytes from immunized mice. Secretion percentages corresponding to the irrelevant peptides were subtracted from values obtained with the relevant peptides. *: differences among values are statistically significant (p≦0.05).

FIG. 5 is an alignment by Clustalw 1.83 of L proteins from HBVs infecting a wide range of animals. Cysteine residues are highlighted in red.

FIG. 6 is a juxtaposition of relevant hydropathy profiles: (A) profile of the amino acid sequence (preS2 region, V3 loop and polyepitope) upstream the HBsAg ATG start codon in the ppolHIV.opt construction; (B and C) superposition of the profiles of the pre-S1/pre-S2 peptides of different hepatitis B viruses: (B) human (D12980, M12906, D00220, X77309 and M32138), gibbon (AAL84829), chimpanzee (AAG4196 and BAB12583), orang-outan (AF193864 and AF193863), and woolly monkey (AA07456); (C) woodchuck (86062931, 8918452, 88101359), and ground-squirrel (84267998).

FIG. 7 depicts the cloned in frame nucleic acid sequence and the deduced amino acid sequence of the polHIV-1.opt polyepitope of the invention.

FIG. 8 is the hydropathy profile of the in frame polHIV1.opt polyepitope of FIG. 7 by DNA Strider™ 1.2.

FIG. 9 depicts the nucleic acid-sequence and the restriction enzyme sequence of a polylinker sequence used in a control plasmid designated pCMV-basic.

FIG. 10 relates to polHIV-1.opt epitope. FIG. 10(A) depicts the nucleotide sequence for polHIV-1.opt. FIG. 10(B) depicts the amino acid sequence of polHIV-1.opt. Epitope numbers are indicated above the sequence. FIG. 10(C) is a hydropathy profile of polHIV-1.opt by DNA Strider™ 1.2.

FIGS. 11(A), 11(B), 11(C), and 11(D) depict the amino acid sequence and hydropathy profile for optimized polyepitopes designated pol1A2, pol2A2, pol1B7, and pol2B7, respectively.

FIG. 12(A) is the nucleic acid sequence from preS2 to HBsAg ATG start codons in the pGA1xFlag-Mpol.opt construction.

FIG. 12(B) is the nucleic acid sequence from preS2 to HBsAg ATG start codons in the pGA3xFlag-Mpol.opt construction.

FIG. 13(A) is the hydropathy profile for the polyepitopic sequence encoded by the nucleic acid sequence of FIG. 12(A).

FIG. 13(B) is the hydropathy profile for the polyepitopic sequence encoded by the nucleic acid sequence of FIG. 12(B).

FIG. 14 is pGA1xFlag-Mpol.opt nucleic acid sequence.

FIG. 15 is pGA3xFlag-Mpol.opt nucleic acid sequence.

In FIGS. 14 and 15, nucleic acid sequences in bold correspond to the following polHIV-1.opt polyepitope amino acid sequence:

YLKEPVHGVRAKTYLNAWVKVVRDTAVLDVGDAYFSVRAKTYLVKLWYQL
RADTRLYNTVATLRTKALLDTGADDTVRAKTLLWKGEGAVRTDAYIYQYM
DDLR

FIG. 16 is pGA1xFlag-M.pol1A2 nucleic acid sequence (in bold: pol1A2 polyepitope).

FIG. 17 is pGA1xFlag-M.pol2A2 nucleic acid sequence (in bold: pol2A2 polyepitope).

FIG. 18 is pGA3xFlag-M.pol1A2 nucleic acid sequence (in bold: pol1A2 polyepitope).

FIG. 19 is pGA3xFlag-M.pol2A2 nucleic acid sequence (in bold: pol2A2 polyepitope).

FIG. 20 is pGA1xFlag-M.pol1B7 nucleic acid sequence (in bold: pol1B7 polyepitope).

FIG. 21 is pGA1xFlag-M.pol2B7 nucleic acid sequence (in bold: pol2B7 polyepitope).

FIG. 22 is pGA3xFlag-M.pol1B7 nucleic acid sequence (in bold: pol1B7 polyepitope).

FIG. 23 is pGA3xFlag-M.pol2B7 nucleic acid sequence (in bold: pol2B7 polyepitope).

FIG. 24 depicts the secretion kinetics corresponding to pGA1xFlag-Mpol.opt and pGA3xFlag-Mpol.opt.

FIG. 25 depicts the secretion kinetics corresponding to pGA1xFlag-Mpol1.A2 and pGA1xFlag-Mpol2.A2.

FIG. 26 depicts the secretion kinetics corresponding to pGA3xFlag-Mpol1.A2 and pGA3xFlag-Mpol2.A2.

FIG. 27 depicts the secretion kinetics corresponding to pGA1xFlag-Mpol1.B7 and pGA1xFlag-Mpol2.B7.

FIG. 28 depicts the secretion kinetics corresponding to pGA3xFlag-Mpol1.B7 and pGA3xFlag-Mpol2.B7.

FIG. 29 provides examples (out of 7⁷) of possible polHIV-1.opt epitope permutations:polyepitope amino acid sequences and corresponding hydropathy profiles (epitope order in the polyepitope is indicated in the polyepitope number as indicated in FIG. 10(B)).

FIG. 30 A: is a schematic representation of the ppolHIV1.opt vector. B depicts the complete nucleotide sequence of ppolHIV1.opt (in bold: nucleic acid sequence corresponding to polHIV1.opt polyepitope).

### DETAILED DESCRIPTION OF THE INVENTION

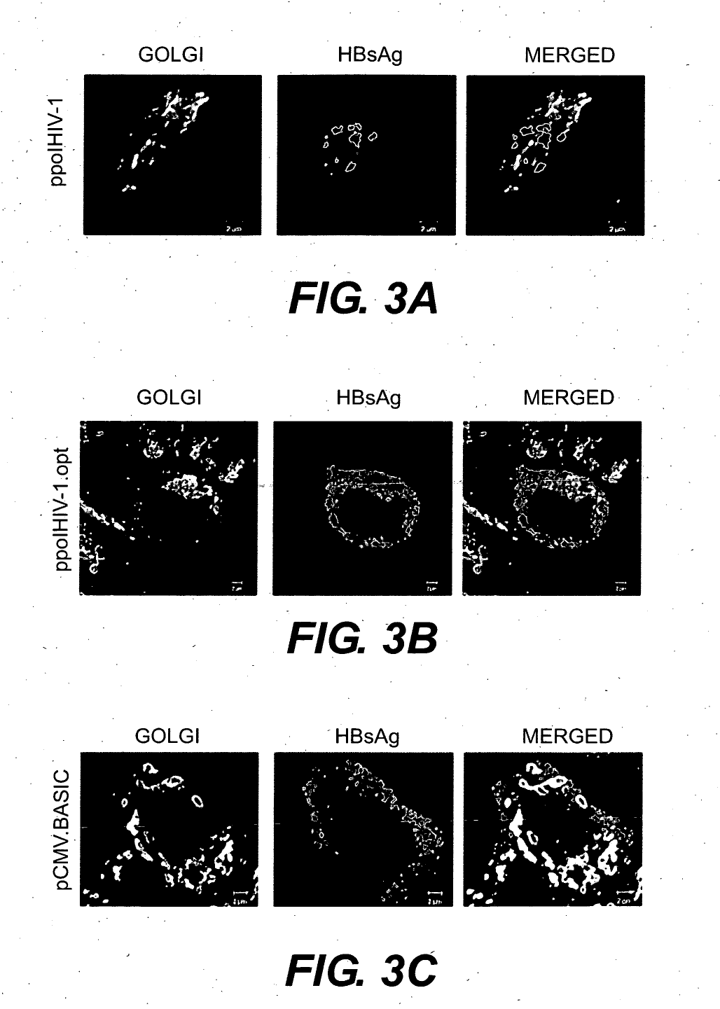

The hepatitis B surface antigen (HBsAg) can assemble into sub-virion virus like particles (VLPs). By fusing immunogenic peptides to the amino-terminus of HBsAg, several bivalent vaccines have been developed. Notably, a polyepitope bearing HIV-1 epitopes restricted to the HLA-A*0201 class I allele elicited a significant HIV-1 specific CD8⁺ cytotoxic T lymphocyte (CTL) response in vivo (12). Inventors of the present patent application have demonstrated that this recombinant HBsAg failed to form VLPs due to retention in the Golgi apparatus (see FIG. 3A).

Inventors of the present patent application have discovered that the polyepitope nucleic and amino acid sequences can be optimized by permutating epitopes in the polyepitope in order to obtain the best hydrophilic profile, counterbalancing the generally hydrophobic class I epitopes with hydrophilic spacers, eliminating epitopes bearing cysteine residues, limiting the number of epitopes with internal methionine residues to a minimum, and optionally adopting Homo sapiens codon usage. In a preferred embodiment of the invention, optimized HIV-1 polyepitope-HBsAg recombinant proteins were assembled into VLPs and efficient secretion of VLPs was achieved.

Further, it has been discovered that DNA immunization in mice results in the induction of humoral neutralizing response against the carrier (HbsAg) and enhanced levels of polyepitope-specific CD8+ T lymphocytes activation.

It is thus possible to make self-assembling recombinant HBsAg VLPs with an heterologous polyepitope, provided a certain number of features typical of naturally occurring preS1 and preS2 regions are respected. This is demonstrated for an HIV-1 polyepitope, and thus provides efficient bivalent HBV/HIV vaccines, which is particularly apposite given that these two viruses are frequently associated.

Thus, this invention employs part or all of the open reading frame (ORF) of the hepatitis B virus envelope gene, which encodes the envelope proteins, each of which begins with an in-frame ATG start codon. The portions of the ORF (proceeding in a 5′ to 3′ direction) and the proteins encoded by them are referred to herein as preS1+preS2+S regions encoding the large (L) envelope protein, preS2+S regions encoding the middle (M) envelope protein, and the S region encoding the major (otherwise known as small) (S) protein identified herein as hepatitis B surface antigen (HBsAg). Thus, HBsAg protein generally means S protein.

The preS1, preS2, and S regions of envelope proteins of different HBV viral isolates may contain several amino acid differences. Some of these differences may lead to changes in antigenicity of the envelope proteins. The regions of the HBV envelope gene employed in practicing this invention can be selected from any of the antigenic subtypes d, y, w, and r. Changes in sequences lead to the generally mutually exclusive d/y and w/r viral subtypes. Thus, it will be understood that the HBsAg virus-like particles of the invention can be based on any of the adw, adr, ayw, or ayr HBV subtypes.

The L, M, and S envelope proteins all are found in varying proportions in the intact HBV virus as well in non-infectious HBV 22 nm particles. In a preferred embodiment of the invention, S envelope proteins form with fusion proteins the basis for the recombinant HBsAg virus-like particles of this invention. In the recombinant HBsAg VLP, L envelope protein is absent because preS1 coding region has been removed from the vector, and M envelope protein as such is no more produced, the major part of preS2 coding region having been removed on behalf of the polylinker and inserted polynucleotide encoding the heterologous polyepitope. Instead of native M envelope protein, recombinant HBsAg VLP contain fusion proteins resulting from inserting in frame a polynucleotide encoding the heterologous polyepitope in preS2 coding region.

More particularly, the recombinant HBsAg virus-like particles of the invention incorporate the S envelope protein of any of the HBV subtypes. The S protein may or may not be fully or partially glycosylated. The nature and extent of glycosylation will depend upon the host cell in which the S region of the HBV envelope gene is expressed and have not been found to be critical in this invention. It will be understood that the recombinant virus-like particles of the invention can incorporate the full length S protein or a truncated form of the S protein, for example, a protein in which N-terminal amino acids, C-terminal amino acids, or both N-terminal and C-terminal amino acids non-essential for particle assembly are deleted. Optionally, the hydrophobic domains of the S protein are retained, and no more than 10 amino acids are deleted from the N-terminal end of the S protein and no more than about 50 amino acids are deleted from the C-terminal end of the S protein. Preferably, the entire S protein is incorporated in the recombinant virus-like particles of the invention.

The recombinant HBsAg virus-like particles of the invention also incorporate at least a portion of the M envelope protein encoded by the preS2 and S coding regions of the envelope gene of any of the HBV subtypes. In a preferred embodiment of the invention, a minimal portion of the N-terminal and C-terminal sequences of preS2 region is encoded. Both have to be in the produced fusion protein: the N-terminal, to ensure translation from the preS2 ATG start codon, and the C-terminal, to ensure to the HBsAg ATG start codon the nucleic context which results in its higher strength, when compared to the preS2 one. The portions of the preS2 region incorporated in the virus-like particles may or may not be fully or partially glycosylated. Once again, the nature and extent of glycosylation will depend upon the host cell in which the preS2 region of the HBV envelope gene is expressed and have not been found to be critical in this invention.

The recombinant HBsAg virus-like particles of the invention thus comprise a mixture of S proteins and fusion proteins where a heterologous polyepitopic sequence is inserted in frame within the preS2 region of M envelope protein. As used herein, the term “heterologous” includes foreign sequences from an organism other than HBV as well as sequences from another protein of HBV. In a preferred embodiment of the HBsAg VLP of the invention, the heterologous polyepitopic sequence is any polyepitopic sequence other than the native epitopic sequence of preS2 region.

Insertion of a polyepitope sequence in the partially deleted preS2 sequence is a preferred embodiment of the invention. Nevertheless, polynucleotides or vectors, where the polyepitope is inserted in a part or all of preS2 region, are also within the scope of the invention. Absence of preS1 region in the nucleic acid construct encoding recombinant HBsAg VLP is also a preferred embodiment of the invention.

The heterologous polyepitopic sequence can contain from 8-11 to 138-140 amino acid residues, preferably from about 20-26 to about 138-140 amino acid residues, especially from about 63-64 to about 138-140 amino acid residues. The polyepitopic sequence is free of cysteine residues and contains as few methionine residues as possible, insofar as they do no disturb the translation efficiency of the preS2 and S ATG start codons. The epitopes in the heterologous polyepitopic sequence are in head-to-tail position.

The heterologous polyepitopic sequence can be constituted of from any number of sequences of interest. The sequence of interest is any sequence other than the sequence of the carrier protein used for the formation of the recombinant VLP of the invention. When HBsAg is employed as carrier protein for formation of recombinant VLP of the invention, sequence of interest can be, for example, an epitopic sequence from other HBV proteins as the capsid protein. The sequence of interest can be an amino acid sequence of any plant, animal, bacterial, viral, or parasitic organism. For example, the sequence of interest can be of a pathogen or of a tumor antigen, such as a human tumor antigen.

The term “pathogen” as used herein, means a specific causative agent of disease, and may include, for example, any bacteria, virus, or parasite. The term “disease” as used herein, means an interruption, cessation, or disorder of body function, system, or organ. Typical diseases include infectious diseases. For example, the polyepitopic sequence can be from the immunogenic proteins of an RNA virus, such as HIV-1, HIV-2, SIV, and HTLV-I, and HTLV-II. Specific examples are the structural or NS1 proteins of Dengue virus; the G1, G2, or N proteins of Hantaan virus; the HA proteins of Influenza A virus; the Env proteins of Friend murine leukemia virus; the Env proteins of HTLV-1 virus; the preM, E, NS1, or NS2A proteins of Japanese encephalitis virus; the N or G proteins of Lassa virus; the G or NP proteins of lymphocytic choriomeningitis virus; the HA or F proteins of measles virus; the F or HN proteins of parainfluenza 3 virus; the F or HN proteins of parainfluenza SV5 virus; the G proteins of Rabies virus; the F or G proteins of respiratory syncytial virus; the HA or F proteins of Rinderpest; or the G proteins of vesicular stomatitis virus.

The polyepitopic sequence can also be from the immunogenic proteins of a DNA virus, such as gp89 of cytomegalvirus; gp340 of Epstein-Barr; gp13 or 14 of equine herpes virus; gB of herpes simplex 1; gD of Herpes simplex 1; gD of herpes simplex 2; or gp50 of pseudorabies.

Further, the polyepitopic sequence can be from the immunogenic proteins of bacteria, such as Streptococci A M6 antigens, or tumor antigens, such as human melanoma p97, rat Neu oncogene p185, human epithelial tumor ETA, or human papillomavirus antigens.

In one embodiment of this invention, the polyepitopic sequence is from a human immunodeficiency virus. Following are HIV-1 epitopes that can be employed in designing the polyepitopic sequence.

| GAG | P17 (77-85) | SLYNTVATL (S9L) |
|---|---|---|
| | P24 (19-27) | TLNAWVKW (T9V) |
| POL | (79-88) | LLDTGADDTV (L10V) |
| | (263-273) | VLDVGDAYFSV (V11V) |
| | (334-342) | VIYQYMDDL (V9L) |
| | (464-472) | ILKEPVHGV (19V) |
| | (576-584) | PLVKLWYQL (P9L) |
| | (669-679) | ESELVNQIIEQ (E11Q) |
| | (671-680) | ELVNQIIEQL (E10 |
| | (956-964) | LLWKGEGAV (L9V) |
| ENV | Gp41 (260-268) | RLRDLLLIV (R9V) |
| NEF | (188-196) | AFHHVAREL (A9L) |

Numbering is based on the amino acid sequence of the HIV-1 WEAU clone 1.60 (Genbank accession no. U21135). The WEAU sequence may not be always identical to that of the reactive peptide and simply indicates its location in the viral proteins.

Epitopes of interest from one or more proteins or polypeptides of one or several different origins are identified and optimized polyepitope is constructed according to the optimization method of the invention. The epitopes are arranged in head-to-tail position. In a preferred embodiment of the invention are chosen epitopic sequences without cysteine and with as few methionine as possible, extra methionine codons being able to initiate translation of truncated fusion proteins and disrupt the translation of HBsAg. The epitopes and the nucleic acids encoding them can be purified from the organism. The epitopes can be alternately synthesized by chemical techniques, or prepared by recombinant techniques.

The polyepitopic sequence thus comprises a multiplicity of epitopes linked to each other in head-to-tail position. It will be understood that the virus-like particles of the invention can contain multiple epitopes of one or several origins, such as epitopes from different immunogenic proteins of the same pathogen or tumor antigen. It will also be understood that the virus-like particles can contain one or more epitopes from different pathogens or tumor antigens. In addition, mixtures of virus-like particles having different epitopes in different particles are contemplated by this invention.

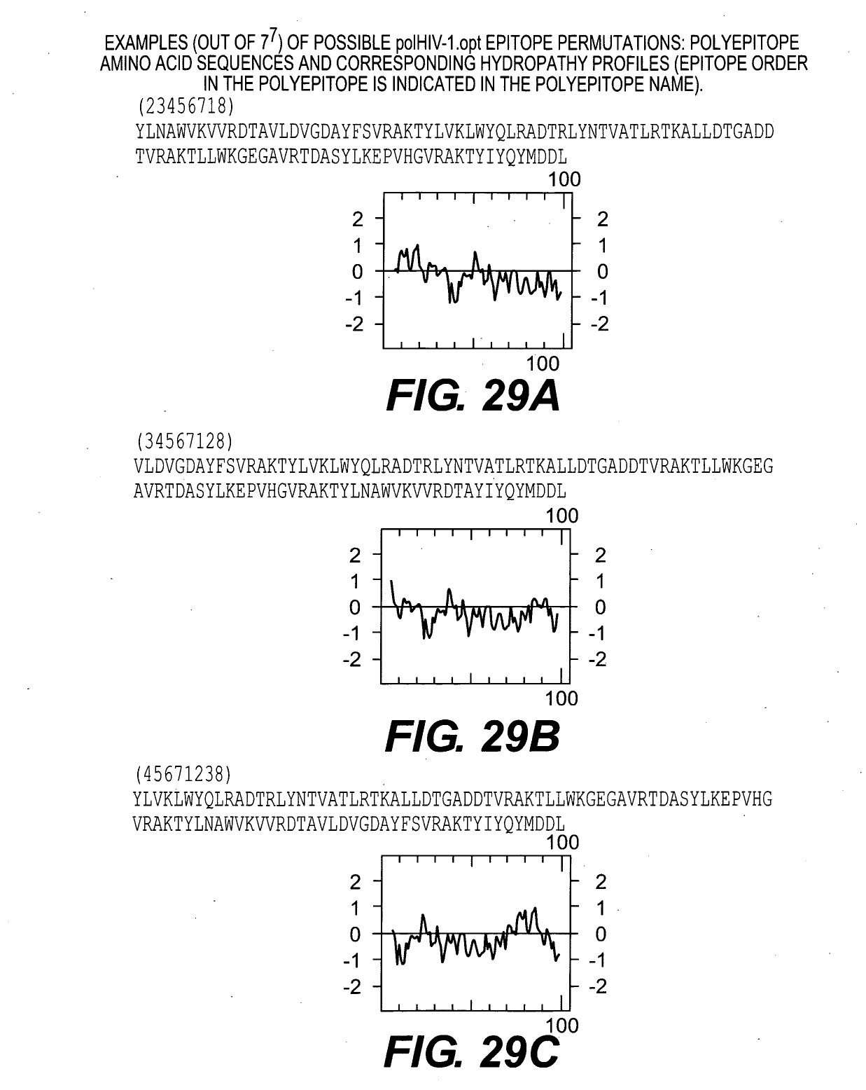

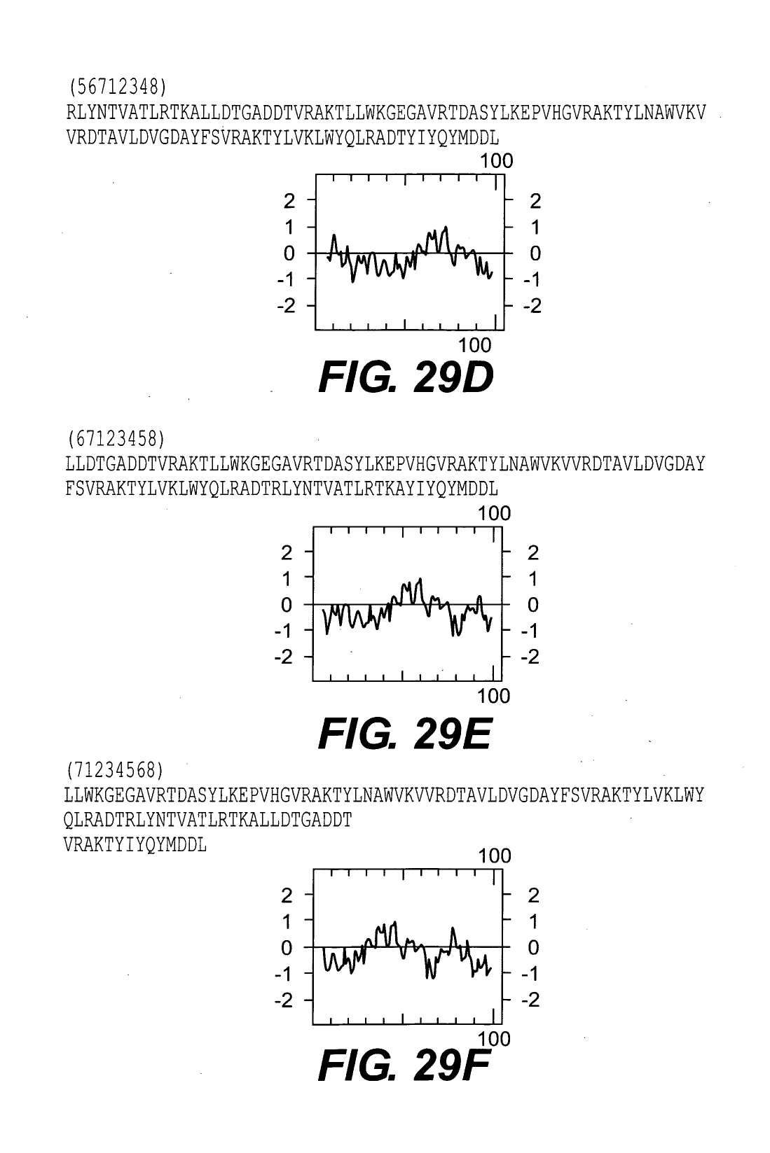

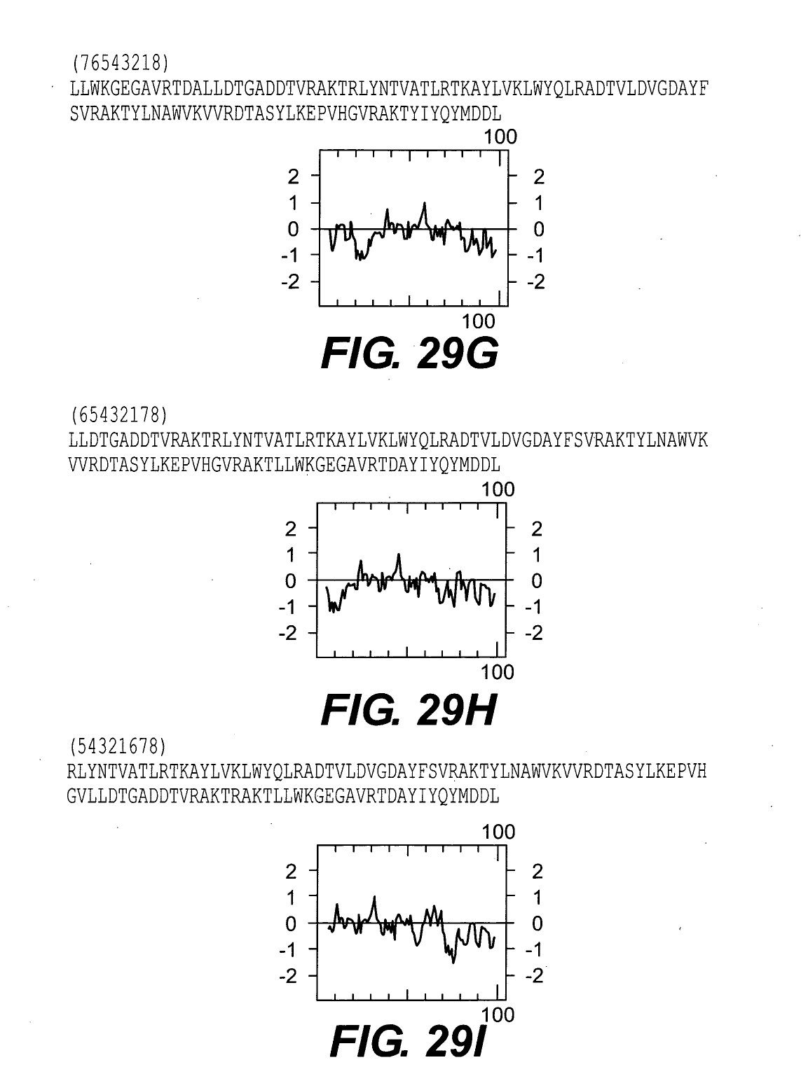

In one embodiment of the invention, the epitopes in a polyepitopic sequence are rearranged so that a new polyepitopic sequence is created in which the order of the epitopes is different from the order of the epitopes in the native or wild sequence from which the new polyepitopic sequence is constructed. The resulting, new polyepitopic sequence contains the epitopes in head-to-tail position. The epitopes can be reordered in this manner to change the hydrophilicityhydropathy profile of the polyepitope. Examples of polyepitopic sequences with reordered epitopes are depicted in FIG. 29.

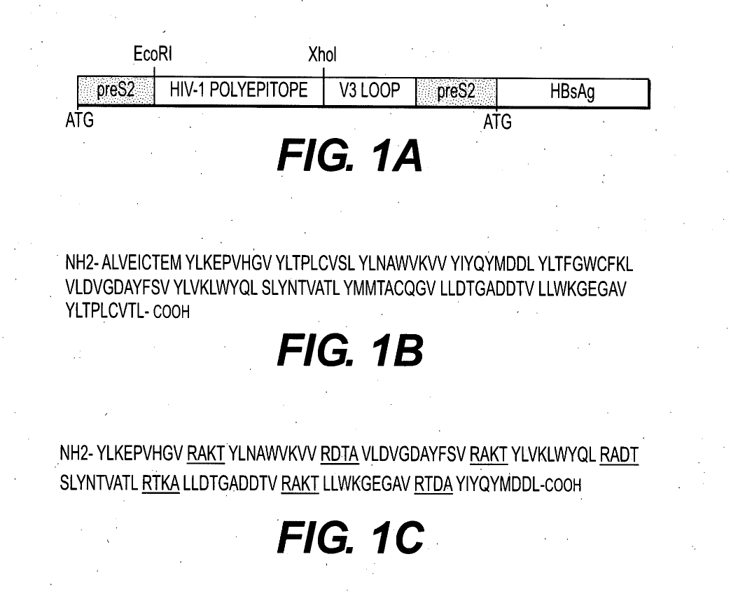

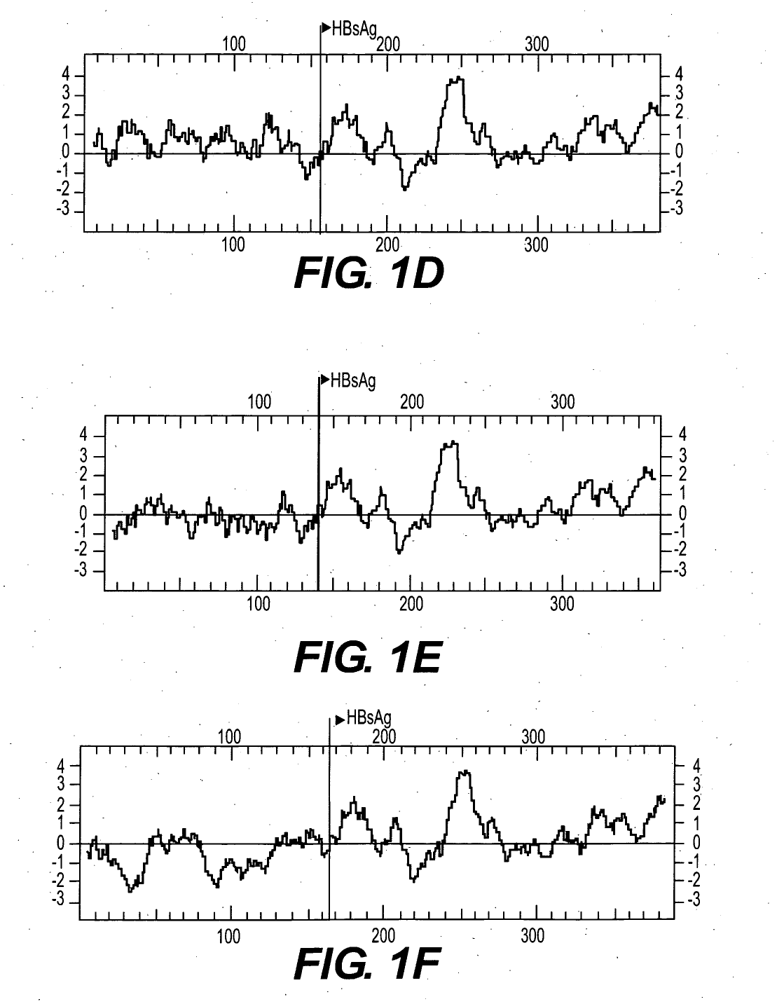

The heterologous polyepitopic sequence containing the epitopes in head-to-tail position is modified by the insertion of tetra-amino acid spacers between the epitopes. Each spacer comprises, for example, an arginine (R) residue placed in the epitope C1-position directly linked to a sequence comprised of three different amino acids, which are independently selected from alanine (A), threonine (T), lysine (K), and aspartic acid (D). An example of an HIV-1 polyepitopic sequence in which the epitopes are interrupted by the tetra-amino acid spacers is depicted in FIG. 1(C). The tetra-amino acid spacers are underlined in this Figure. Permutation of residues which follow arginine was made to avoid at nucleic acid level repeated homologous sequence along the complete gene which could impair correct gene synthesis by using techniques based on polymerization (like PCR). At the amino acid level, the only aim is to increase hydrophilicity of the polyepitope, hence residues order is not important in itself. Furthermore, the choice of A, T, K and D is not exclusive. Other hydrophilic amino acids such as serine (S), glutamine (Q), asparagine (N) and histidine (H) might as well be used in their place.

The heterologous polyepitopic sequence containing the epitopes interrupted by spacers is positioned within the preS2 region of M envelope protein. The polynucleotide coding for the heterologous polyepitopic sequence is inserted in preS2 coding region such that translation from preS2 and S (also named HBsAg) ATG start codons is preserved so that two proteins are produced, the two ATG start codons being preserved in their natural nucleic acid context. The first protein is S (also named HBsAg). The second protein is a fusion protein comprised of the heterologous polyepitopic sequence within the preS2 region of the M envelope protein. Together, the HBsAg protein and the fusion protein assemble into the virus-like particles of the invention after expression in an eukaryotic host cell.

The location of the polyepitopic sequence in the preS2 region can be readily determined. This invention is based on the following requirements: 1) preservation of natural nucleic acid context around preS2 and HBsAg ATG start codons (−6 to +3, being A in the ATG=0); 2) preservation of the preS2 glycosylation site: NST in the initial amino acid sequence MQWNST; and, 3) reduction to the minimum amino acid sequence in length of preS2 region, to give space to polyepitopic sequence to be inserted. In a preferred embodiment of the invention, preS2 region is partially deleted while fulfilling the above requirements.

The immunodominant epitope of preS2 needs not to be preserved.

In a preferred embodiment of the invention the virus-like particles lack detectable L protein.

It will be understood that the recombinant virus-like particles of the invention can contain subunits, such as truncated copies, of the HBsAg and the fusion proteins. The subunits may be produced, for example, by variation in gene expression and protein processing in the host cell, or by initiation of translation from an ATG codon contained in the polynucleotide encoding the heterologous polyepitope.

The HBsAg proteins can assemble with host cell derived lipids into multimeric particles that are highly immunogenic in comparatively low concentrations. The fusion protein containing the heterologous polyepitope is exposed on the surface of the recombinant virus-like particles of the invention. Thus the recombinant virus-like particles provide excellent configurational mimics for protective epitopes as they exist in their native context, such as an infectious virus. For these reasons, the recombinant virus-like particles of the invention are suitable for exploitation as carriers for protective determinants of other etiologic agents. These highly immunogenic virus-like particles display the heterologous epitopes while retaining the protective response to HBV determinants.

The immune response will depend upon the heterologous polyepitope and can be an antibody response imparting humoral immunity, neutralizing antibody response, such as protective humoral immunity. The term “humoral immunity” or “humoral immune response” as used herein, means antibodies elicited by an antigen, and all the accessory processes that accompany it. The term “protective humoral immunity” as used herein, means a humoral immune response that confers the essential component of protection based on neutralizing antibodies directed against a pathogen. Suitable methods of antibody detection include, but are not limited to, such methods as ELISA, immunofluorescence (IFA), focus reduction neutralization tests (FRNT), immunoprecipitation, and Western blotting.

The immune response can also be manifest as antibody-dependent cell-mediated cytotoxicity (ADCC), delayed-type hypersensitivity (DTH), cytotoxic T cell response, or helper T cell response. The recombinant virus-like particles of the invention are thus suitable for use as immunogens or vaccines, depending upon the nature of the immune response in the host species.

Recombinant expression vectors prepared in accordance with the present invention make it possible to obtain a cell-mediated immune response, especially a cytotoxic T lymphocytes (CTL) reaction against epitopes of the heterologous polyepitope. This cell-mediated immune response can be a specific response, obtained against one or several epitopes encoded by the recombinant expression vectors.

Since the highly immunogenic recombinant virus-like particles of the invention display the heterologous epitopes while retaining the protective response to HBV determinants, the recombinant virus-like particles of the invention and the recombinant expression vectors encoding them can be employed as mono-vaccine candidates, double vaccine candidates, or as immunization agents producing two or more immune responses, depending upon the identity of the different epitopes of the heterologous polyepitope displayed by the recombinant virus-like particles.

Target antigens have been identified in several types of tumors and in particular in melanomas or in carcinomas, including renal carcinomas, bladder carcinomas, colon carcinomas, lung carcinomas, breast cancer, leukemia and lymphoma. Therefore, the invention provides a means for use in treatment protocols against tumors and cancer and especially for use in protocols for immunotherapy or vaccination therapy against tumors. The invention also provides means for the treatment or prophylaxis of infectious diseases, especially diseases associated with virus infection, for instance, with retrovirus infection. The cell-mediated immune response, and especially the CTL response associated with the treatment by a composition comprising the recombinant expression vectors of the invention or/and the recombinant virus-like particles of the invention, herein referred as the composition of the invention, can be specific for the tumor antigen or of the virus or virus infected cells, and can also be restricted to specific molecules of the MHC. Particularly, the invention relates to the use of the recombinant expression vector of the invention in an immunogenic composition in order to obtain a cell-mediated immune response restricted to Class I molecules of the MHC complex, and for instance restricted to the HLA-A2 or -B7 alleles.

In one aspect, the invention is directed to recombinant HBsAg virus-like particles, which deliver HIV epitopes. Advantageously, the recombinant virus-like particles of the invention are capable of inducing an in vitro, ex vivo, and/or in vivo CTL response against HIV in a mammal. More particularly, the immunogenic recombinant virus-like particles according to the invention can induce in vitro, ex vivo and/or in vivo specific cytotoxic CD8 T-lymphocytes (CTLs) capable of eliminating specifically HIV-infected cells. The present invention thus relates to polyepitopes from HIV proteins, and more particularly from the Gag, Pol, Env, Vif, Tat, Vpu, Rev, Vpr, Vpx, and Nef proteins of HIV-1 and HIV-2. The invention also relates to polynucleotides coding for the polyepitopes.

The nucleic acid construct encoding the recombinant virus-like particles of the invention can be inserted in a variety of different types of expression vectors for a host cell. The resulting vectors are herein referred to as the recombinant expression vectors of the invention. These vectors include vectors for use in eukaryotic expression systems and preferably for mammalian expression systems, such as recombinant poxvirus expression vectors, for example, vaccinia virus, fowlpox virus, or canarypox virus; animal DNA viruses, for example, herpes simplex 1 and 2, varicella zoster, pseudorabies, human cytomegalovirus, murine cytomegalovirus, Esptein-Barr virus, Karposi's sarcoma virus, or murine herpes virus. Animal RNA viruses can also be employed as vectors for expression of the nucleic acid construct of the invention. Suitable animal RNA viruses include positive-strand RNA viruses, such as the picornaviruses, for example, poliovirus, the flaviviruses, for example, hepatitis C virus, or coronaviruses. Examples of other suitable vectors are lentiviral vectors, adenoviral vectors, and adeno-associated viral vectors. Other suitable eukaryotic vectors are expression vectors for yeast cells, expression vectors for insect cells, such as baculoviruses, or even expression vectors for plant cells. Plasmid and phage vectors can also be employed.

The recombinant expression vectors of the invention can be prepared using well known methods. For a review of molecular biology techniques see: Sambrook, et al. Molecular Cloning: A Laboratory Manual, CSH Press 1989. The expression vectors can include the polynucleotide sequence encoding the heterologous polyepitope, “operably linked” to suitable transcriptional or translational regulatory nucleotide sequences, such as those derived from a mammalian, microbial, viral, plant or insect gene. Examples of regulatory sequences include transcriptional promoters, operators, or enhancers, an mRNA ribosomal binding site, and appropriate sequences that control transcription and translation initiation and termination. Nucleotide sequences are “operably linked” when the regulatory sequence functionally relates to the polynucleotide sequence coding for the polyepitope. The ability to replicate in the desired host cells, usually conferred by an origin of replication, and a selection gene by which transformants are identified can additionally be incorporated into the expression vector. In addition, sequences encoding appropriate signal peptides that are not naturally associated with the polyepitopic sequence can be incorporated into the expression vector.

Suitable host cells for expression include yeast or higher eukaryotic cells. Appropriate cloning and expression vectors for use with plant, fungal, yeast, and mammalian cellular hosts are described, for example, in Pouwels et al. Cloning Vectors: A Laboratory Manual, Elsevier, N.Y., (1985).

Introduction of the recombinant expression vector of the invention into the host cell can be effected by calcium phosphate transfection, DEAE-dextran mediated transfection, cationic lipid-mediated transfection, electroporation, transduction, infection, gene transfer, such as OGM generation, e.g., plant OGM, or other methods. Such methods are described in many standard laboratory manuals, such as Davis et al., Basic Methods In Molecular Biology (1986).

Therefore, the invention is also concerned with cells, such as recombinant eukaryotic cells, infected, transformed, or transfected by any of the recombinant expression vectors described above for expressing the recombinant HBsAg virus-like particles of the invention. Methods for producing such cells and methods for using these cells in the production of proteins/peptides are well known in the art.

The invention also relates to cells, which have been put in contact with the recombinant HBsAg virus-like particles according to the invention, and especially relates to recombinant cells containing the recombinant expression vector of the invention. These cells are advantageously antigen presenting cells. As examples, these cells can be chosen among lung cells, brain cells, epithelial cells, astrocytes, mycroglia, oligodendrocytes, neurons, muscle, hepatic, dendritic, neuronal cells, cell strains of the bone marrow, macrophages, fibroblasts, and hematopoietic cells.

In one embodiment of this invention, autologous dendritic cells are loaded ex vivo with the recombinant HBsAg virus-like particles of the invention or recombinant expression vectors of the invention encoding the particles. The resulting dendritic cells can be employed for immunizing a host. The dendritic cells can be used as a primer source of immunization or a booster source of immunization.

In another aspect, the invention is directed to a method for producing, in vitro, recombinant HBsAg virus-like particles according to the invention, comprising: culturing in vitro, in a suitable culture medium, a cell incorporating a recombinant expression vector of the invention, and collecting in the culture medium HBsAg virus-like particles produced by these recombinant cells. The virus-like particles are released from the host cell into the extracellular space.

The invention provides immunogenic recombinant HBsAg virus-like particles, and more particularly, immunogenic fusion proteins for use in the preparation of vaccine compositions against a variety of diseases. These particles can thus be employed as bacterial, viral, or fungal vaccines by administering the particles to an animal, preferably a mammal, susceptible to infection by the pathogen. These particles can also be employed as immunotherapy or vaccination therapy drug by administering the particles to an animal, preferably a mammal having a tumor.

Conventional modes of administration can be employed. For example, administration can be carried out by oral, respiratory, or parenteral routes. Intradermal, subcutaneous, and intramuscular routes of administration are preferred when the vaccine is administered parenterally. Intramuscular administration is particularly preferred.

The mammals can be, for example, humans, other primates, such as chimpanzees and monkeys, or bovines, ovines, porcines and equines, such as horses, cows, pigs, goats, sheep, or dogs, cats, chickens, rabbits, mice, hamsters, or rats. The mammal is preferably a human.

Effective quantities of the recombinant HBsAg virus-like particles of the invention can be administered with an inert diluent or carrier. They can be combined with the following ingredients: a binder, such as microcrystalline cellulose, gum tragacanth, or gelatin; an excipient, such as starch or lactose; a disintegrating agent, such as alginic acid, corn starch, and the like; a lubricant, such as magnesium stearate; a glidant, such as colloidal silicon dioxide; a liquid carrier, such as a fatty oil. Other dosage unit forms can contain various materials that modify the physical form of the dosage unit, for example, as coatings. Materials used in preparing these various compositions should be pharmaceutically pure and non-toxic in the amounts used.

The ability of the recombinant HBsAg virus-like particles and vaccines of the invention to induce protective humoral immunity in a host can be enhanced by emulsification with an adjuvant, incorporating in a liposome, coupling to a suitable carrier, or by combinations of these techniques. In a preferred embodiment, the recombinant HBsAg virus-like particles of the invention can be administered with a conventional adjuvant, such as aluminum phosphate and aluminum hydroxide gel, in an amount sufficient to potentiate humoral or cell-mediated immune response in the host. Similarly, the recombinant HBsAg virus-like particles can be bound to lipid membranes or incorporated in lipid membranes to form liposomes. The use of nonpyrogenic lipids free of nucleic acids and other extraneous matter can be employed for this purpose.

The recombinant HBsAg virus-like particles and vaccines of the invention can be administered to the host in an amount sufficient to prevent or inhibit pathogen infection. In any event, the amount administered should be at least sufficient to protect the host, even though infection may not be entirely prevented.

An immunogenic response can be obtained by administering the recombinant HBsAg virus-like particles of the invention to the host in an amount of about 5-40 micrograms per dose by intramuscular injection in a subject. The dose depends upon whether the recipient is an infant, a child, an adolescent, or an adult, and also upon the health of the recipient. The recombinant HBsAg virus-like particles of the invention can be administered together with a physiologically acceptable carrier. For example, a diluent, such as water or a saline solution, can be employed.

The immunization schedule will depend upon several factors, such as the susceptibility of the host to infection and the age of the host. A single dose of the recombinant HBsAg virus-like particles of the invention can be administered to the host or a primary course of immunization can be followed in which several doses at intervals of time are administered. Subsequent doses used as boosters can be administered as needed following the primary course.

A preferred dosing schedule is comprised of separate doses at timed intervals. For example, a preferred dosing schedule for human subjects comprises a first dose at an elected date, a second dose one month later, and a third dose six months after the first dose. Booster doses or revaccination can be employed, for example, 12 and 24 months later.

Another aspect of the invention provides a method of DNA vaccination. The method includes administering the recombinant expression vectors encoding the recombinant HBsAg virus-like particles, per se, with or without carrier molecules, to the subject.

Thus, the methods of treating include administering immunogenic compositions comprising recombinant HBsAg virus-like particles, or compositions comprising a polynucleotide encoding recombinant HBsAg virus-like particles as well. Those of skill in the art are cognizant of the concept, application, and effectiveness of nucleic acid vaccines (e.g., DNA vaccines) and nucleic acid vaccine technology, as well as protein and polypeptide based technologies. The nucleic acid based technology allows the administration of a polynucleotide encoding HBsAg virus-like particles, naked or encapsulated, directly to tissues and cells without the need for production of encoded proteins prior to administration. The technology is based on the ability of this polynucleotide to be taken up by cells of the recipient cell or organism and expressed to produce an immunogenic protein to which the recipient's immune system responds. Typically, the expressed antigens are displayed on the surface of cells that have taken up and expressed the polynucleotide, but expression and export of the encoded antigens into the circulatory system of the recipient individual is also within the scope of the present invention. Such nucleic acid vaccine technology includes, but is not limited to, delivery of recombinant expression vectors encoding recombinant HBsAg virus-like particles. Although the technology is termed “vaccine”, it is equally applicable to immunogenic compositions that do not result in a protective response. Such non-protective inducing compositions and methods are encompassed within the present invention.

Although it is within the present invention to deliver a polynucleotide encoding recombinant HBsAg virus-like particles and carrier molecules, the present invention also encompasses delivery of polynucleotides as part of larger or more complex compositions. Included among these delivery systems are complexes of the invention's virus-like particles with cell permeabilizing compounds, such as liposomes.

The present invention further relates to antibodies that specifically bind the recombinant HBsAg virus-like particles of the invention. The antibodies include IgG (including IgG1, IgG2, IgG3, and IgG4), IgA (including IgA1 and IgA2), IgD, IgE, or IgM. As used herein, the term “antibody” (Ab) is meant to include whole antibodies, including single-chain whole antibodies, and antigen-binding fragments thereof. The antibodies can be human antigen binding antibody fragments, and include, but are not limited to, Fab, Fab′ and F(ab′)2, Fd, single-chain Fvs (scFv), single-chain antibodies, disulfide-linked Fvs (sdFv), and fragments comprising either a V_{L} or V_{H} domain. Fab and F(ab′)2 fragments can be produced by proteolytic cleavage, using enzymes, such as papain (to produce Fab fragments) or pepsin (to produce F(ab′)2 fragments). The antibodies can be from any animal origin. Preferably, the antibodies are human, murine, rabbit, goat, guinea pig, camel, horse, or chicken.

Antibodies of the present invention have uses that include, but are not limited to, methods known in the art to purify, detect, and target the recombinant HBsAg virus-like particles of the invention, including both in vitro and in vivo diagnostic and therapeutic methods. For example, the antibodies have use in immunoassays for qualitatively and quantitatively measuring levels of the particles of the invention in biological samples. See, e.g., Harlow et al., ANTIBODIES: A LABORATORY MANUAL, (Cold Spring Harbor Laboratory Press, 2nd ed. 1988) (incorporated by reference in the entirety).

The antibodies of the present invention can be prepared by any suitable method known in the art. For example, recombinant HBsAg virus-like particles of the invention can be administered to an animal in order to induce the production of sera containing polyclonal antibodies. Monoclonal antibodies can be prepared using a wide of techniques known in the art, including the use of hybridoma and recombinant technology. See, e.g., Harlow et al., supra, Hammerling, et al., in: MONOCLONAL ANTIBODIES AND T-CELL HYBRIDOMAS 563-681 (Elsevier, N.Y., 1981) (incorporated by reference in their entireties).

While this invention relates to recombinant HBsAg virus-like particles carrying one or more heterologous polyepitopes on their surfaces, this invention also provides a method for optimizing the polyepitopes to be carried on virus-like particles. As an example, the surface antigen (HBsAg) of the Hepatitis B virus (HBV) carries all the information required for membrane translocation, particle assembly, and secretion from mammalian cells. HBsAg assembles into VLPs polymeric structure that enhances antigenic stability. It is only if assembled in VLPs that HBsAg can be secreted out of cells. In this system, secretion provides high-density HBsAg presentation to antigen presenting cells (APCs). This invention provides criteria for optimizing the polyepitope sequence, which ensure the conservation of recombinant virus-like particle structure and secretion, once the virus-like particle is used as carrier of a polyepitope. These parameters are:
1) Overall hydrophilicity of the polyepitope, the more hydrophylic, the better;
2) the introduction of small hydrophilic amino acid spacers between epitopes to increase the overall hydrophilicity of the polyepitope; a preferred spacer is a tetra-amino acid spacer, and the amino acids are chosen preferably among arginine, alanine, threonine, lysine, aspartic acid, serine, glutamine, asparagine and histidine, and more preferably among arginine, alanine, threonine, lysine and aspartic acid;
3) the absence of methionine residues or limitation to only ones that are of comparable or less strength to that of preS2 translation initiation one and that belong to immunodominant epitope, in this later case the epitope is placed at the C-terminal region of the polyepitope
4) the absence of cysteine residues; and
5) optionally, codon usage optimization according to the organism in which the polyepitope has to be expressed.

This invention provides also criteria for optimizing the polyepitope sequence, which ensure the optimal epitope processing and higher level of immunogenicity. These criteria are
1) head-to-tail positioning of epitopes; and
2) introduction at the epitope C1-terminal position of the small spacer a basic, amide or small residue, an arginine (R) residue being the preferred to promote the processing of the epitopes and increase their immunogenicity.

Thus, the method of this invention for optimizing the polyepitopic sequence of interest for incorporation in a virus-like particle, such as HBsAg VLPs, comprises providing a polynucleotide sequence encoding a polyepitopic sequence of interest, wherein the polyepitopic sequence comprises cysteine and methionine codons and is hydrophobic; removing the codons for cysteine and the codons for methionine; and providing polynucleotides encoding small hydrophilic spacers between the epitopes in the polyepitopic sequence. Each spacer comprises preferably an arginine residue placed in the epitope C₁-position directly linked to a sequence of three different amino acids independently selected from, for example, alanine, threonine, lysine, and aspartic acid. The method further comprises optimizing codon usage in the polyepitopic sequence based on preferred codon usage patterns in the Homo sapiens genome. The method can further comprise head-to-tail positioning of epitopes sequences in the polyepitopic sequence.

It will be understood that this invention also provides an optimized polynucleotide sequence and an optimized polyepitopic (amino acid) sequence encoded by the optimized polynucleotide sequence.

This invention provides for optimization of polyepitope at two levels, namely, VLPs secretion and epitope processing. The invention thus includes the method of optimization, an optimized polyepitope and the polynucleotide encoding it, the vector and the virus-like particle from VLPs secretion, and alternatively or optionally, epitope processing. The characteristics “head-to-tail epitopes” and “presence of an R residue in the epitope C1 position” are not directly implicated in VLP secretion, so that it will be understood that these are optional features of the invention. Similarly, while the “tetra amino acid spacers” are described as part of the invention, it will be understood that small hydrophilic amino acid spacers can be employed. With respect to the characteristic “0 or 1 codon for methionine” in the polynucleotide coding for the heterologous polyepitope, the goal is to eliminate all the internal methionine codons by selecting epitopes without methionine codons. An exception has been made for an immunodominant epitope that contained a methionine codon, which has been localized at the C-terminal end of the polyepitope. The reason of this location is that, even if translation is initiated from this internal ATG codon, it will produce truncated fusion proteins similar to HBsAg.

A “polynucleotide” also includes those polynucleotides capable of hybridizing, under stringent hybridization conditions, to the optimized polynucleotide sequences of the invention, the complement thereof, or the DNA within a deposit. “Stringent hybridization conditions” refers to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 mug/ml denatured, sheared salmon sperm DNA, followed by washing the filters in 0.1×SSC at about 65° C.

Also contemplated are polynucleotides that hybridize to the optimized polynucleotide sequences of the invention at moderately high stringency hybridization conditions. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC).

The optimized polyepitopic amino acid sequences of the invention can be used to generate fusion proteins. For example, the optimized polyepitopic amino acid sequence, when fused to a second protein, can be used as an antigenic tag. Antibodies raised against the optimized sequence can be used to indirectly detect the second protein by binding to the optimized sequence. Domains that can be fused to optimized sequence include not only heterologous signal sequences, but also other heterologous functional regions. The fusion does not necessarily need to be direct, but may occur through linker sequences.

Moreover, fusion proteins can also be engineered to improve characteristics of the optimized polyepitopic amino acid sequence of the invention. For instance, a region of additional amino acids, particularly charged amino acids, may be added to the N-terminus of the optimized sequence to improve stability and persistence during purification from the host cell or subsequent handling and storage. Also, peptide moieties can be added to the optimized sequence to facilitate purification. Such regions can be removed prior to final preparation of the optimized sequence. The addition of peptide moieties to facilitate handling of polypeptides are familiar and routine techniques in the art.

Moreover, the optimized polyepitopic amino acid sequence of the invention can be combined with parts of the constant domain of immunoglobulins (IgG), resulting in a chimeric polypeptide. This fusion protein show an increased half-life in vivo. A fusion protein having disulfide-linked dimeric structures (due to the IgG) can also be more efficient in binding other molecules, than the monomeric secreted protein or protein fragment alone. In many cases, the Fc part in a fusion protein is beneficial in therapy and diagnosis, and thus can result in, for example, improved pharmacokinetic properties.

The optimized polyepitopic amino acid sequence and the fusion protein containing it can be recovered and purified from recombinant cell cultures by well known methods, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Preferably, high performance liquid chromatography (“HPLC”) is employed for purification.

In a preferred embodiment, this invention provides a fusion protein comprised of the optimized polyepitopic sequence positioned within the partially deleted preS2 region of an HBV M protein and a nucleotide sequence encoding the fusion protein.

The optimized nucleic acid sequence and the optimized polyepitopic amino acid sequence of the invention have been optimized for a HBsAg carrier for the formulation of VLPs. It will be understood, however, that other carriers can be employed for the VLPs of the invention. For example, genetically engineered chronic HBV/HEV virus-like particles can be employed. See Clin. Med. Sci. J. 2004; 19(2); 78-83. Also, HBC and frCP virus-like particles can be used. See Intervirology 2002; 45(1); 24-32. Also World J. Gastronterol. 2005; 11(4); 492-97. Similarly, yeast Ty virus-like particles can be employed. See Yeast 2000; 16(9); 785-95. Further, it will be understood that parvovirus-like particles can be utilized. See Proc. Natl. Acad. Sci. USA 1997; 94(14); 7503-8. In addition, HPV pseudovirus can be employed as a carrier for VLPs. See Methods Mol. Med. 2005; 119; 445-62. VLP composed of Capsid protein of Norwalk and Norwalk-like viruses can also be employed as VLP of the invention. See Proc. Natl. Acad. Sci. USA 1996; 93(11); 5335-40. The entire disclosure of each of these publications is relied upon and incorporated by reference herein.

Following the criteria of the invention, several optimized polyepitopic sequences of HIV-1 were prepared for incorporation in the recombinant HBsAg virus-like particles of the invention, and the resulting particles were assayed for activity.

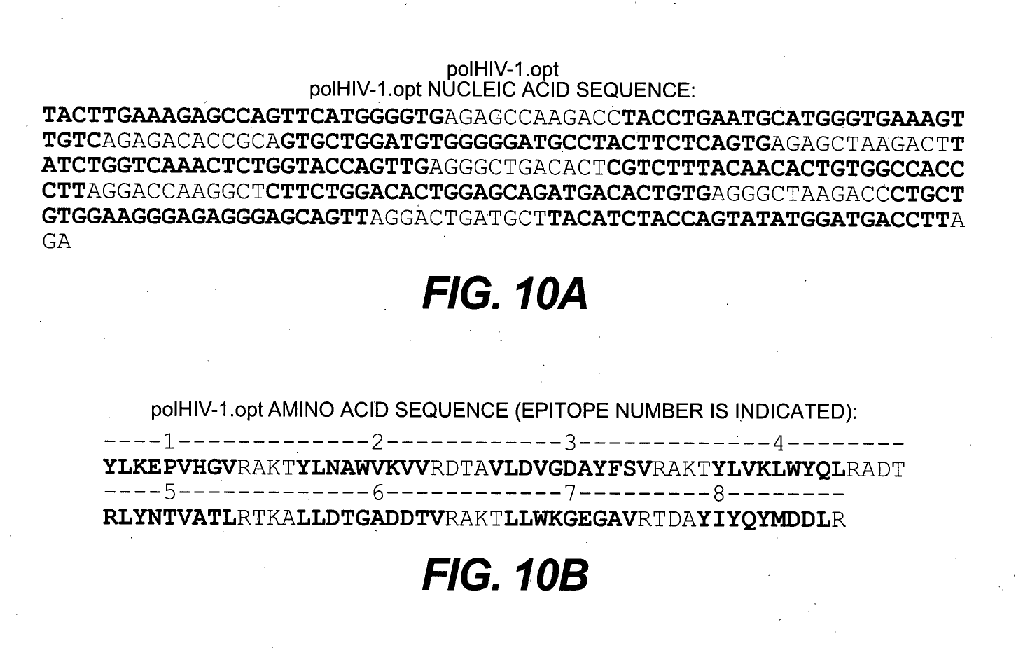

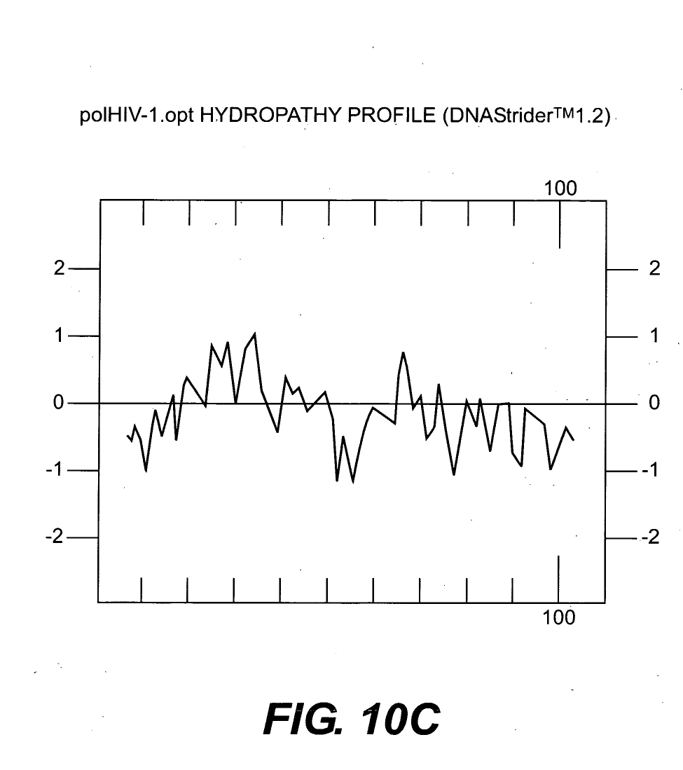

The first optimized polyepitope was designated polHIV-1.opt. The nucleic acid sequence and amino acid sequence of polHIV-1.opt are shown in FIGS. 10A and 10B. The amino acid sequences of polHIV-1 polyepitope described in FIGS. 1C and 10B are not exactly the same. The difference is in the arginine (R) residue at the C-terminal end in sequence of FIG. 10B. This residue (and corresponding codon) was added to the raw sequence of polyepitope to promote the processing of the last C-terminal epitope. The sequence of FIG. 10 can be then considered as the most optimized polHIV-1 opt polyepitope according to the criteria provided by the invention.

The hydropathy profile (DNAStrider™1.2) for polHIV-1.opt is shown in FIG. 10C.

More particularly, following the optimization criteria, the polHIV-1.opt polyepitope of the invention was synthesized by multiple rounds of “atypical” PCR, as described in the following Examples, and using the long primers detailed in the Table 1.

TABLE 1

Oligonucleotides used for the
polHIV-1.opt polyepitope construction

| | |
|---|---|
| Oligonucleotide | Sequence |

| | |
|---|---|
| HIVPOLY-1 | 5′GAATTCCTACTTGAAAGAGCCAGTTCATGGGG |
| | TGAGAGCCAAGACCTACCTGAATGCATGGGTGAA |
| | AGTTG |
| HIVPOLY-2 | 5′CTGAATGCATGGGTGAAAGTTGTCAGAGACAC |
| | CGCAGTGCTGGATGTGGGGGATGCCTACTTCTCA |
| | GTGAGAG |
| HIVPOLY-3 | 5′ATGCCTACTTCTCAGTGAGAGCTAAGACTTAT |
| | CTGGTCAAACTCTGGTACCAGTTGAGGGCTGACA |
| | CTCG |
| HIVPOLY-4 | 5′CAGTTGAGGGCTGACACTCGTCTTTACAACAC |
| | TGTGGCCACCCTTAGGACCAAGGCTCTTCTGGAC |
| | ACTGGAGCAGATG |
| HIVPOLY-5 | 5′CTTCTGGACACTGGAGCAGATGACACTGTGAG |
| | GGCTAAGACCCTGCTGTGGAAGGGAGAGGGAGCA |
| | GTTAGGACTG |
| HIVPOLY-6 | 5′AAGGGAGAGGGAGCAGTTAGGACTGATGCTTA |
| | CATCTACCAGTATATGGATGACCTTAGACTCGAG |
| 5′compmodifpe | 5′CATGAACTGGCTCTTTCAAGTAGGAATTCCAC |
| | TG |
| 5′modifpoly | 5′GCAGTGGAATTCCTACTTGAAAGAGCCAGTTC |
| | ATG |
| 5′modifpoly | 5′CATATATGCTCGAGTCTAAGGTCATCCATATA |
| | CTG |

Nucleic and amino acid sequences and corresponding polHIV-1.opt hydropathy profiles are given in FIGS. 10A, 10B and 10C, respectively.

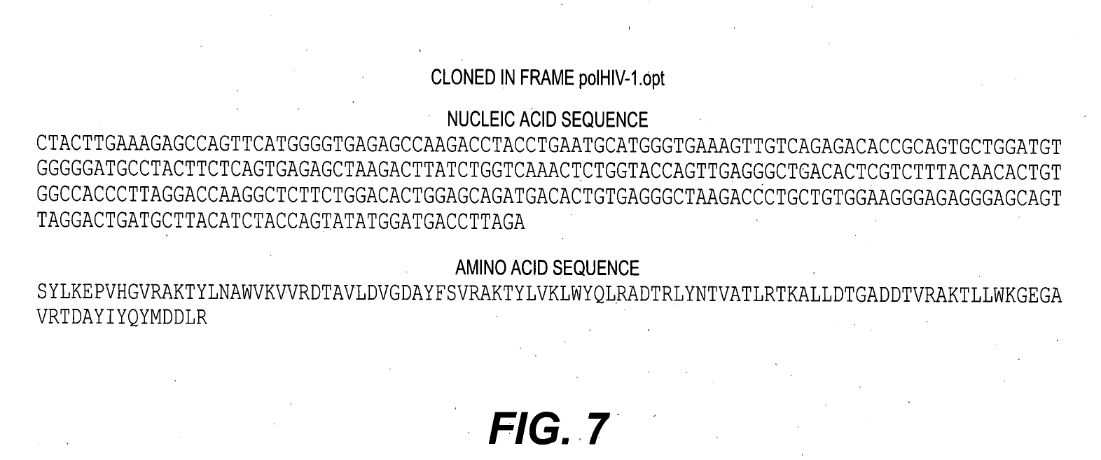

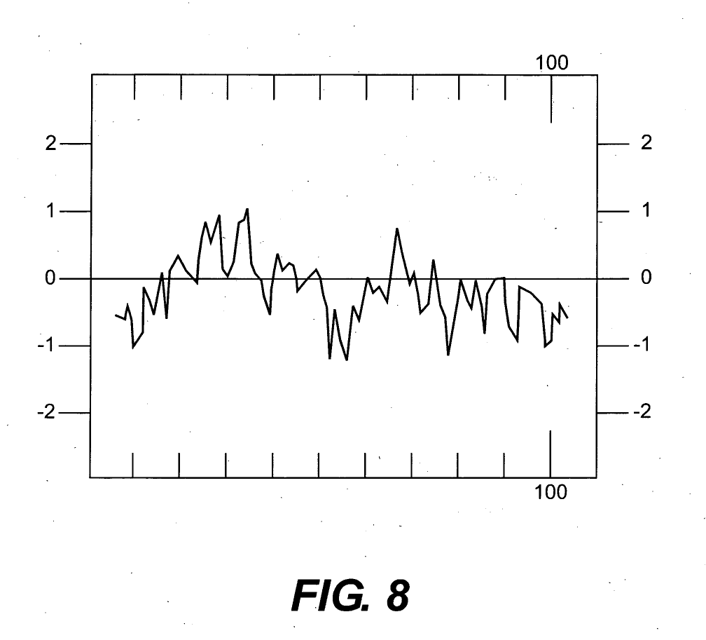

The polHIV-1.opt polyepitope was cloned in frame (FIGS. 7 and 8) in between the EcoRI and XhoI restriction sites of the pCMV-B10 polylinker, (Marsac et al., (2005), In vivo induction of cellular and humoral immune response by hybrid DNA vectors encoding simian/human immunodeficiency virus/hepatitis B surface antigen virus particles in BALB/c and HLA-A2-transgenic mice, Immunobiology 210:305-319; and Le Borgne et al., (1998) In vivo induction of specific cytotoxic T lymphocytes in mice and rhesus macaques immunized with DNA vector encoding an HIV epitope fused with hepatitis B surface antigen, Virology 240:304-315), giving the ppoHIV-1.opt plasmid construction described in the following Examples. In this construction, the preS2 N-terminal and C-terminal portions, which have been conserved in the pCMV-B10 plasmid, surround the polHIV-1.opt polyepitope, which is fused at the C-terminal extremity to the HIV-1 V3 loop, used as tag. This construct is depicted in FIG. 1.

The sequence of the polHIV-1 opt polyepitope shown in FIG. 7 is the sequence of the polyepitope as cloned in the pCMV-B10 and pGA1xFlagM vectors. The nucleic acid sequence contains an extra C nucleotide at 5′ end compared to the sequence of polHIV-1 polyepitope of FIGS. 1C and 10. The reason is the need of cloning in frame the polyepitope sequence within the preS2 sequence to obtain a fusion protein.

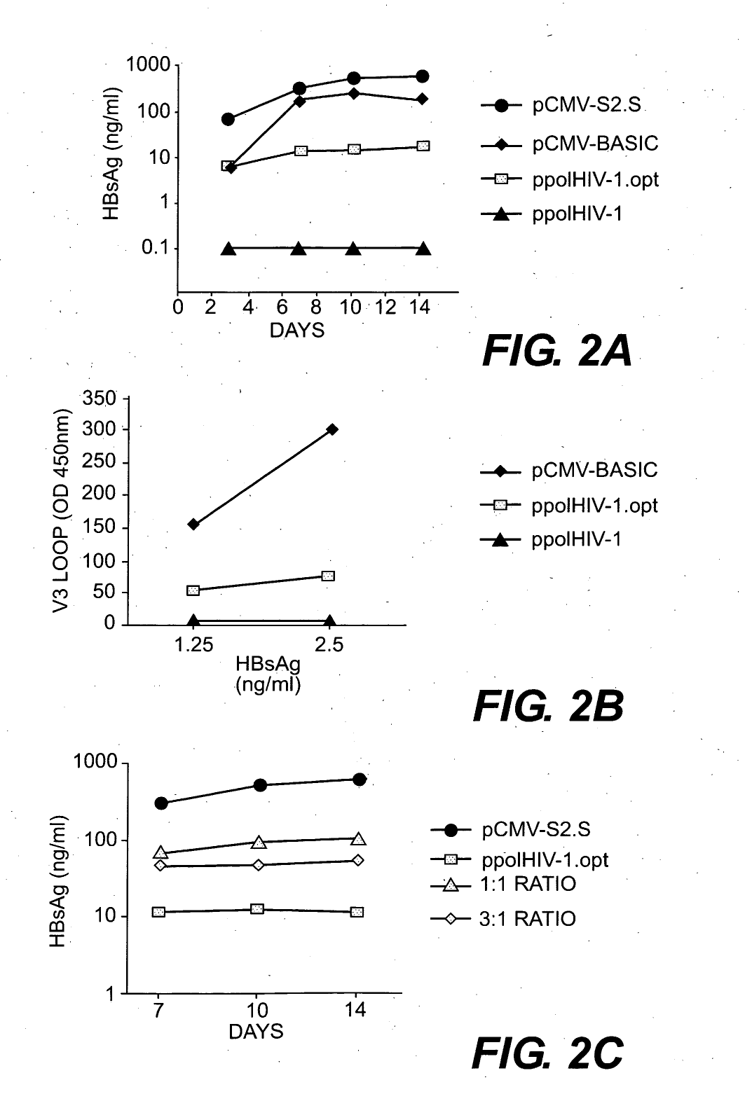

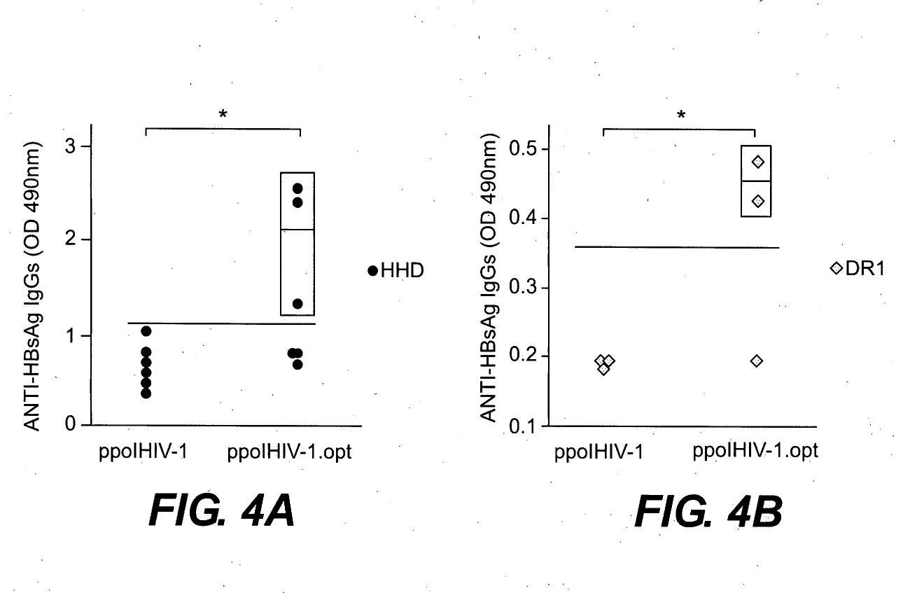

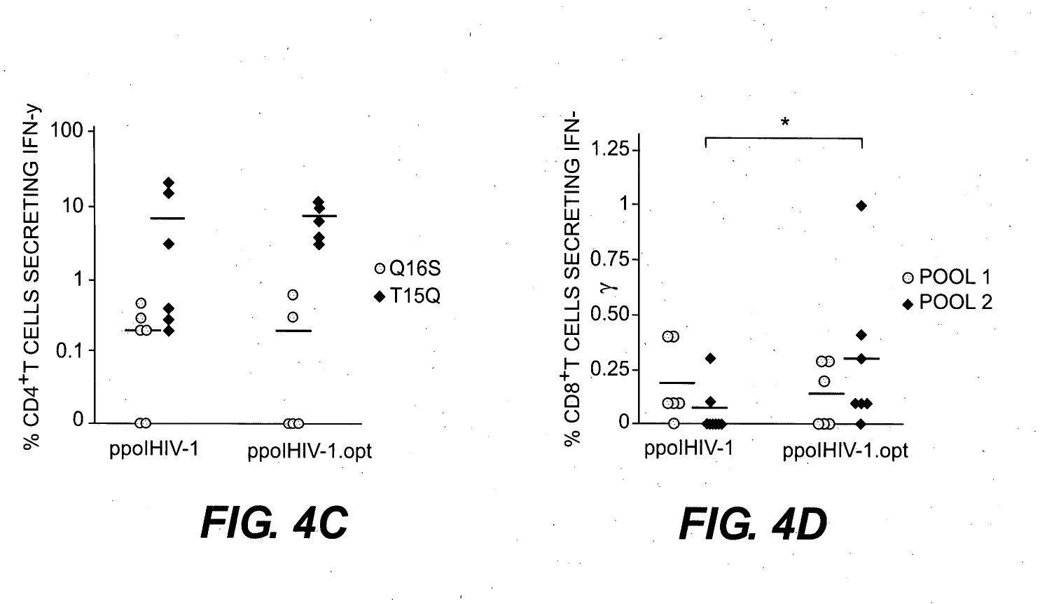

In vitro transient transfection of the SW480 cell line followed by anti-HBsAg and anti-V3 loop ELISA tests made it possible to demonstrate that by optimising the previously described HIV-1 polyepitope, (Bruss, V. (2004) Envelopment of the hepatitis B virus nucleocapsid, Virus Res. 106:199-209), recombinant HBsAg VLPs secretion could be significantly rescued. These results are depicted in FIG. 2 and discussed below. Moreover, by immunising HHD and HLA.A2.1/DRB1 transgenic mice, it was demonstrated that restoration of recombinant HBsAg VLPs secretion could rescue anti-HBsAg humoral response and enhance global HIV-1 specific T lymphocytes activation. These results are shown in FIG. 4. See panels A, B, and D.

The nucleic acid sequence of polHIV-1.opt is depicted in FIG. 10(A) and is as follows:

polHIV-1.opt nucleic acid sequence
TACTTGAAAGAGCCAGTTCATGGGGTGAGAGCCAAGACCTACCTGAATGC
ATGGGTGAAAGTTGTCAGAGACACCGCAGTGCTGGATGTGGGGGATGCCT
ACTTCTCAGTGAGAGCTAAGACTTATCTGGTCAAACTCTGGTACCAGTTG
AGGGCTGACACTCGTCTTTACAACACTGTGGCCACCCTTAGGACCAAGGC
TCTTCTGGACACTGGAGCAGATGACACTGTGAGGGCTAAGACCCTGCTGT
GGAAGGGAGAGGGAGCAGTTAGGACTGATGCTTACATCTACCAGTATATG
GATGACCTTAGA

The nucleic acid sequences encoding eight epitopes of polHIV-1.opt and the corresponding names of the epitopes are shown in Table 2.

TABLE 2
polHIV-1.opt epitopes nucleic acid sequences

| epitope | | |
|---|---|---|
| number | name | corresponding nucleotide sequence |
| 1 | Y/I9V | TACTTGAAAGAGCCAGTTCATGGGGTG |
| 2 | Y/T9V | TACCTGAATGCATGGGTGAAAGTTGTC |
| 3 | V11V | GTGCTGGATGTGGGGGATGCCTACTTCTCAGTG |
| 4 | Y/P9L | TATCTGGTCAAACTCTGGTACCAGTTG |
| 5 | R/S9L | CGTCTTTACAACACTGTGGCCACCCTT |
| 6 | L10V | CTTCTGGACACTGGAGCAGATGACACTGTG |
| 7 | L9V | CTGCTGTGGAAGGGAGAGGGAGCAGTT |
| 8 | Y/V9L | TACATCTACCAGTATATGGATGACCTT |

The corresponding amino acid sequence for each of these epitopes is shown in FIG. 10(B) and is as follows:

polHIV-1.opt amino acid sequence (epitope number
is indicated)
----1-------------2------------3-------------4----
YLKEPVHGVRAKTYLNAWVKVVRDTAVLDVGDAYFSVRAKTYLVKLWYQL
--------5-------------6------------7---------8----
RADTRLYNTVATLRTKALLDTGADDTVRAKTLLWKGEGAVRTDAYIYQYM
----
DDLR.

The epitope number is indicated over the polHIV-1.opt amino acid sequence above. The hydropathy profile is shown in FIG. 10(C).

More particularly, Table 3 shows the origin, position, and frequency of each of these epitopes in HIV-1 genomes.

TABLE 3
polHIV-1.opt epitopes

| | | | | % frequencies |
|---|---|---|---|---|
| | | | | in HIV-1 clade |
| name | sequence | origin | position | A, B and C |
| R/S9L | RLYNTVATL | gag (p17) | 77-85 | 56, 46, 33 |
| L9V | LLWKGEGAV | pol (integrase) | 19-27 | 89, 100, 93 |
| L10V | LLDTGADDTV′ | pol (protease) | 79-88 | 100, 100, 87 |
| V11V | VLDVGDAYFSV | pol (RT) | 263-273 | 89, 90, 93 |
| Y/V9L | YIYQYMDDL | pol (RT) | 334-342 | 33, 93, 87 |
| Y/P9L | YLVKLWYQL | pol (RT) | 464-472 | 89, 100, 93 |
| Y/I9V | YLKEPVHGV | pol (RT) | 576-584 | 11, 76, 80 |
| Y/T9V | YLNAWVKVV | gag (p24) | 956-964 | 11, 83, 20 |

FIG. 29 provides examples of polHIV-1.opt epitope permutations and corresponding polyepitopes hydropathy profiles (epitope order in the polyepitope is indicated in the polyepitope name).

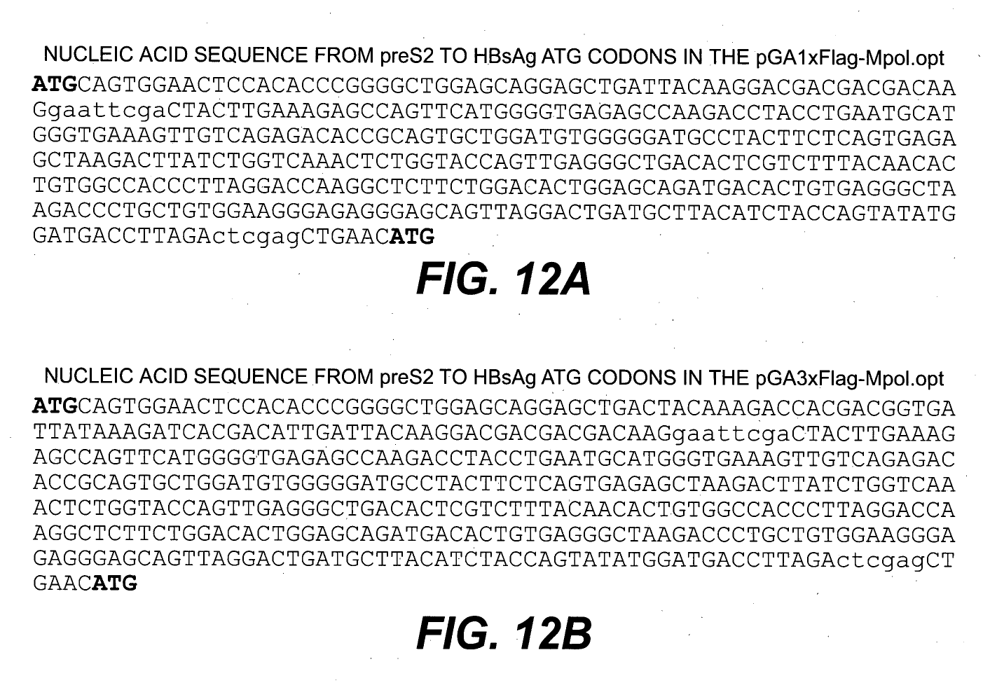

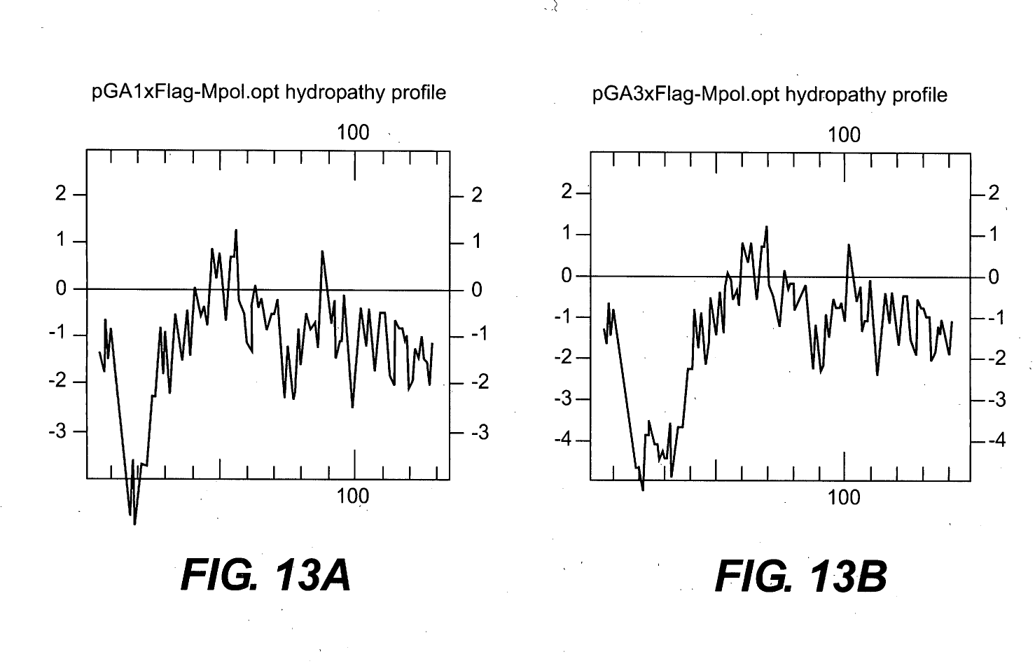

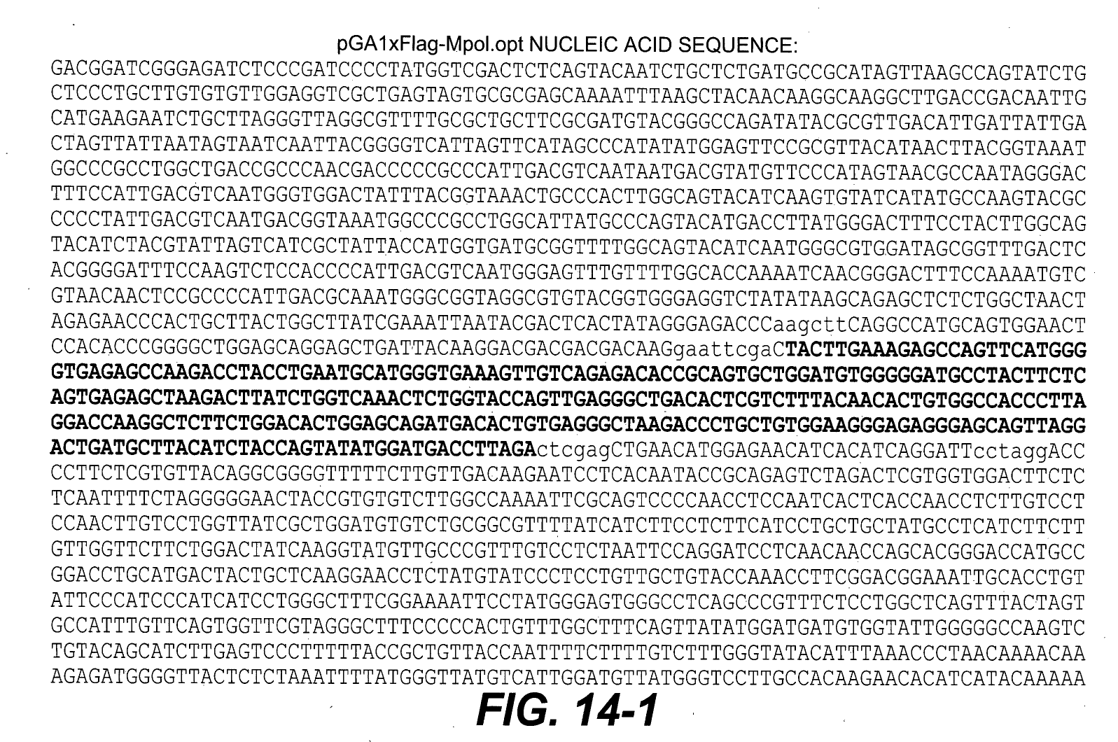

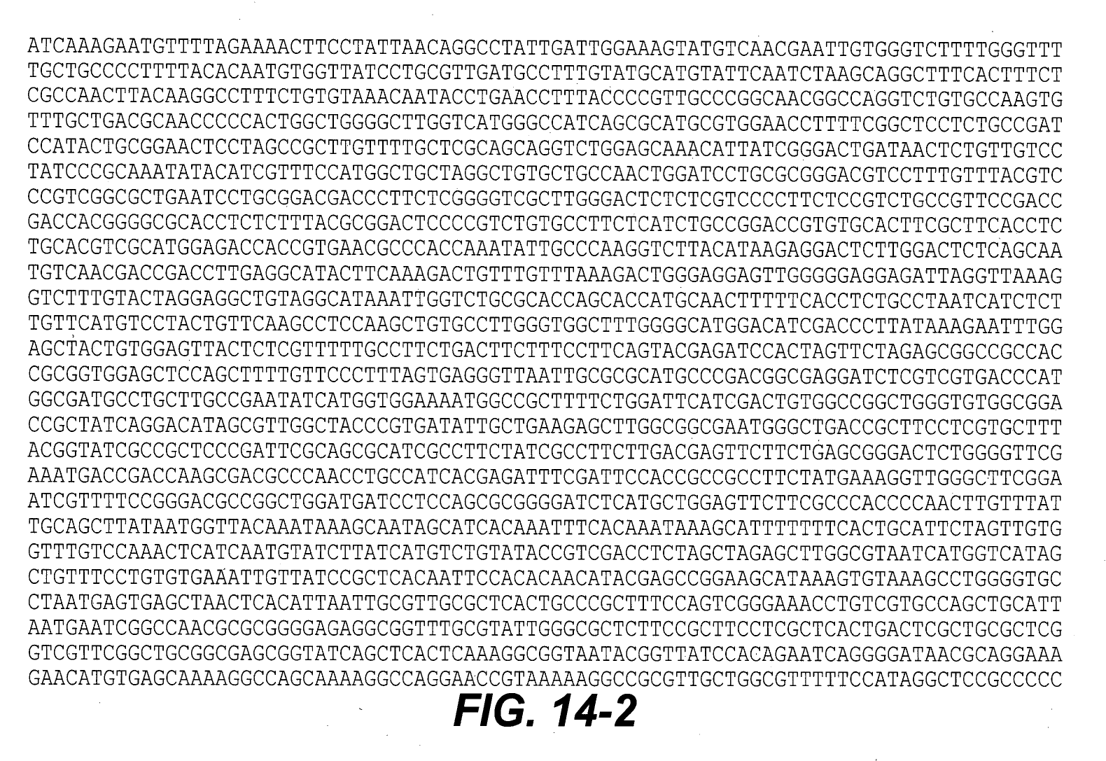

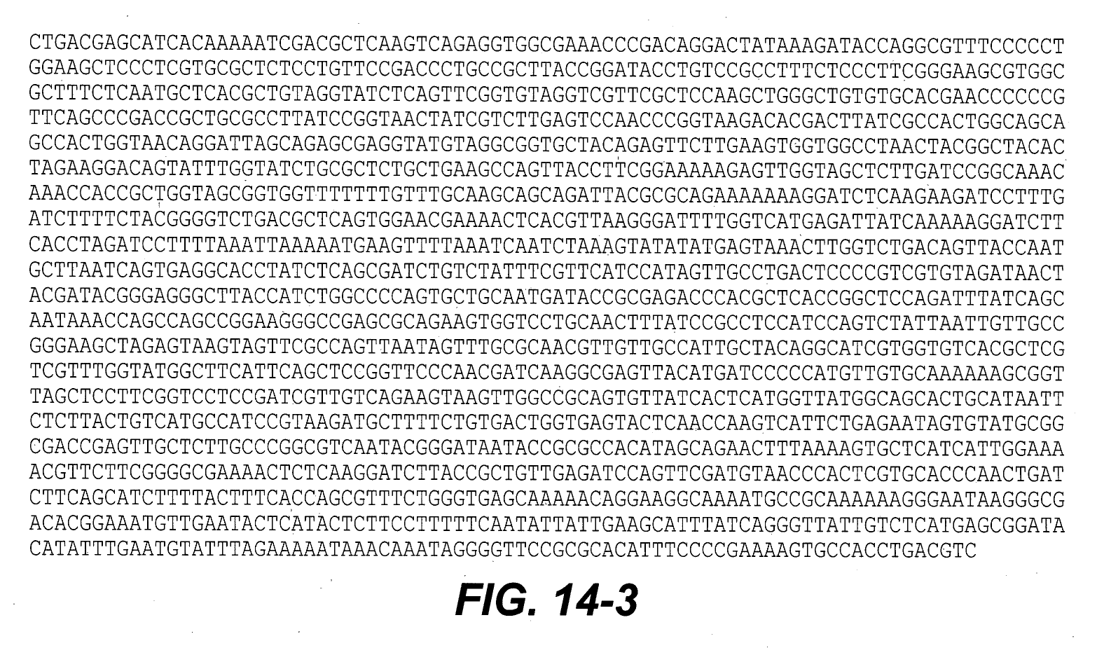

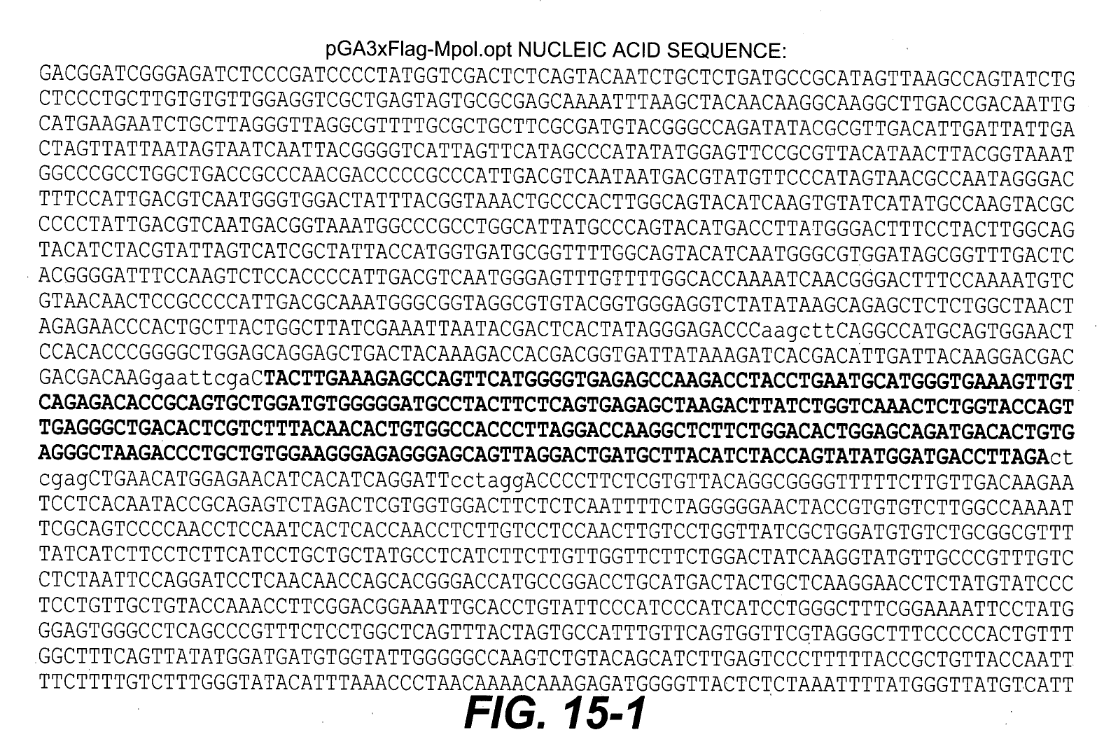

The polHIV-1.opt epitope of the invention was inserted into plasmid pGA1xFlag-M and plasmid pGA3xFlag-M between the preS2 and HBsAg ATG start codons in each plasmid. FIGS. 12(A) and 12(B), FIG. 14 and FIG. 15, show nucleic acid sequences of resulting pGA1xFlag-Mpol.opt and pGA3xFlag-Mpol.opt, respectively. The hydropathy profile for each sequence is shown in FIGS. 13(A) and 13(B), respectively.

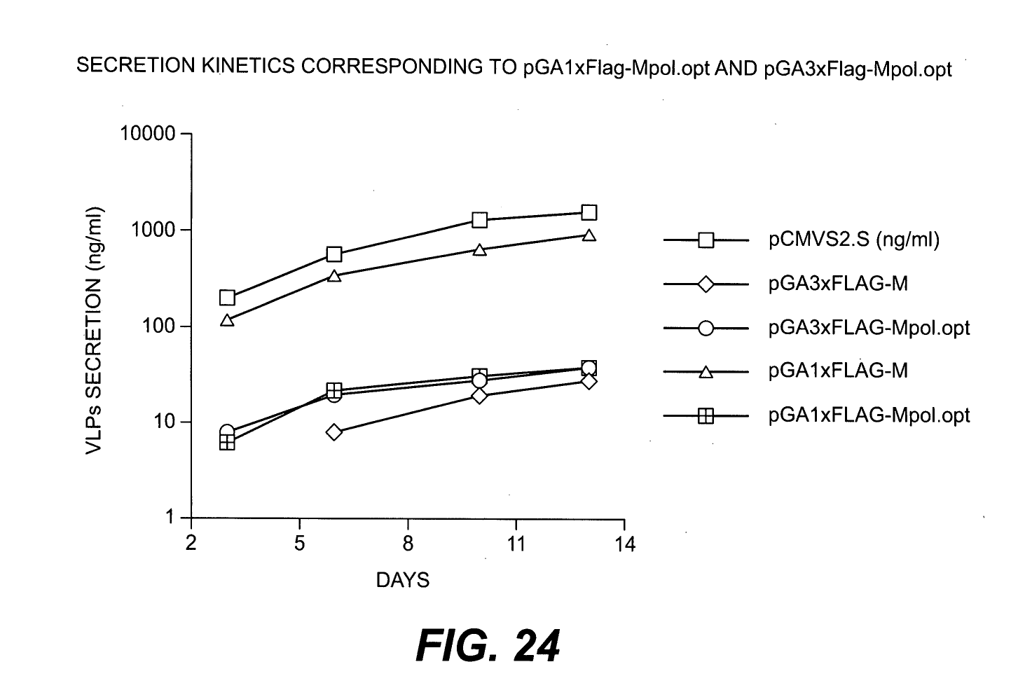

The secretion kinetics corresponding to pGA1 xFlag-Mpol.opt and pGA3xFlag-Mpol.opt are shown in FIG. 24.

Similarly, following the optimization criteria of the invention, four additional optimized polyepitopic sequences were designed. These polyepitopic sequences have been designated pol1A2, pol2A2, pol1B7, and pol2B7. The polyepitopic sequences designated pol1A2 and pol2A2 are assembled from the epitopes in Table 4.

TABLE 4

Listing of A2 epitopes assembled into
pol1A2 (italic) and po12A2 (bold)

| | | | Conservation (%) | Responder/tested | |
|---|---|---|---|---|---|
| origin | protein | name | sequence | (Clade A, B and C) | (HHD mice orHLA-A2.1 tg) |
| Gag | p17 | S9L | SLYNTVATL | 56, 46, 33 (32) | 5/5 |
| | p24 | Y/T9V | YLNAWVKVV | 11, 83, 20 (39) | 3/6 |
| Pol | protease | L10V | LLDTGADDTV | 100, 100, 87 (98) | 4/6 |
| | RT | V11V | VLDVGDAYFSV | 89, 90, 93 (56) | 5/6 |
| | | Y/V9L | YIVQYMDDL | 33, 93, 87 (78) | 2/6 |
| | | Y/19V | YLKEPVHGV | 11, 76, 80 (61) | 1/6 |
| | | Y/P9L | YLVKLWVQL | 89, 100, 93 (86) | 1/6 |
| | intégrase | L9V | LLWKGEGAV | 89, 100, 93 (97) | 5/5 |
| Gag* | j.p24/p2 | Gag 362 | VLAEAMSQV | 100, 74, 13 (52) | 3/6 |
| Vif* | vif | Vif 23 | SLVKHHMYV | 60, 16, 61 (26) | 4/6 |

Repartition of the HLA-A2 allele:
Caucasian population, 25%
Black population, 16%
Oriental population, 27%
Corbel S., Nielsen H. V. et al Optimisation and immune recognition of multiple novel conserved HLA-A2, human immunodeficiency virus type 1-CTL specific epitopes. JGV (2003) 84, 2409-2421

The polyepitopic sequences designated pol1B7 and pol2B7 are assembled from the epitopes in Table 5.

TABLE 5

Listing of B7 epitopes assembled into pol1B7 (italic)
and po12B7 (bold): F10LR is in both constructions

| | | | | Conservetion (%) | Responder/tested |
|---|---|---|---|---|---|
| origin | protein | name | sequence | (Clade A, B and C) | (HLA-B7 mice) |
| gag | p24 | S9WV | SPRTLNAWV | 100, 94, 80 (92) | 5/6 |
| gag | P24 | T9ML | TPQDLNTML | 11, 94, 100 (68) | 2/6 |
| gag-arfp | P24 | Q9VF | QPRSDTHVF | X, 90, X (74) | Detection in |
| (alternative ORF) | | | | | human (1/2) |
| gag | p24 (CyPA | gag 237 | HPVHAGPIA | 0, 74, 38 (56) | Elispot OK |
| | binding domain) | | | | |
| env | gp120 | R10SI | RPNNNTRKSI | 25, 37, 18 (26) | 3/6 |
| env | gp120 | A10VV | APTKAKRRVV | 41, 72, 46 (32) | 2/6 |
| env | gp120 | I9GL | IPRRIRQGL | 12, 32, 14 (21) | 5/6 |
| env | gp120 | K10LL | KPVVSTQLLL | 65, 9, 82 (30) | — |
| nef | nef | F10LR | [symbol] | 79, 70, 82 (73) | — |

Repartition ot the HLA-B7 allele:
Caucasian population, 8.67%
Black population, 7.71%
All epitopes are from Sylvain Cardinaud, except the gag 237 which is from Wilson et al 2003: Jimmunol 171:5611-5623

The nucleic acid and amino acid sequences, as well as epitope name and epitope sequences, are as follows.

Nucleic and amino acid sequences of pol1A2

GTGCTGGATGTGGGAGATGCCTACTTCTCAGTGAGAGCTGACACCTACCT
GAATGCCTGGGTGAAGGTGGTCAGAGCCAAGACCTACCTGGTGAAGCTGT
GGTACCAGCTGAGGACAGATGCCTCCCTGGTGAAGCATCACATGTATGTG
AGAGACACAGCCTACATCTACCAGTACATGGATGACCTGAGA
VLDVGDAYFSVRADTYLNAWVKVVRAKTYLVKLWYQLRTDASLVKHHMYV
RDTAYIYQYMDDLR

| | | |
|---|---|---|
| Name | aa seq | nuc seq |

| | | |
|---|---|---|
| V11V | VLDVGDAYFSV | GTGCTGGATGTGGGAGATGCCTACTTCTCAGT |
| | | G |
| Y/T9V | YLNAWVKVV | TACCTGAATGCCTGGGTGAAGGTGGTC |
| Y/P9L | YLVKLWYQL | TACCTGGTGAAGCTGTGGTACCAGCTG |
| Vif23 | SLVKHHMYV | TCCCTGGTGAAGCATCACATGTATGTG |
| Y/V9L | YIYQYMDDL | TACATCTACCAGTACATGGATGACCTG |

Nucleic and amino acid sequences of pol2A2

CTGCTTGACACAGGAGCTGATGACACAGTGAGGACAGATGCCAGCCTGTA
TAACACAGTGGCCACCCTGAGAGCTGACACCTACCTGAAGGAGCCTGTGC
ATGGAGTGAGAGCTAAGACCCTCCTGTGGAAGGGAGAGGGAGCAGTGAGA
ACCAAGGCAGTGCTGGCTGAGGCCATGTCCCAGGTGAGA
LLDTGADDTVRTDASLYNTVATLRADTYLKEPVHGVRAKTLLWKGEGAVR
TKAVLAEAMSQVR

| | | |
|---|---|---|
| Name | aa seq | nuc seq |

| | | |
|---|---|---|
| L10V | LLDTGADDTV | CTGCTTGACACAGGAGCTGATGACACAGTG |
| S9L | SLYNTVATL | AGCCTGTATAACACAGTGGCCACCCTG |
| Y/I9V | YLKEPVHGV | TACCTGAAGGAGCCTGTGCATGGAGTG |
| L9V | LLWKGEGAV | CTCCTGTGGAAGGGAGAGGGAGCAGTG |
| Gag362 | VLAEAMSQV | GTGCTGGCTGAGGCCATGTCCCAGGTG |

Nucleic and amino acid sequences of pol1B7

TCCCCTAGGACCCTGAATGCCTGGGTGAGAGCTAAGACCAGACCTAACAA
TAACACAAGGAAGTCCATCAGAGACACAGCCTTCCCTGTGAGACCACAGG
TGCCTCTGAGGAGAACCAAGGCCCACCCTGTGCATGCTGGCCCTATTGCC
AGAGCTGATACAGCACCCACTAAGGCCAAAAGGAGAGTGGTCAGG
SPRTLNAWVRAKTRPNNNTRKSIRDTAFPVRPQVPLRRTKAHPVHAGPIA
RADTAPTKAKRRVVR

| | | |
|---|---|---|
| Name | aa seq | nuc seq |

| | | |
|---|---|---|
| S9WV | SPRTLNAWV | TCCCCTAGGACCCTGAATGCCTGGGTG |
| R10SI | RPNNNTRKSI | AGACCTAACAATAACACAAGGAAGTCCATC |
| F10LR | FPVRPQVPLR | TTCCCTGTGAGACCACAGGTGCCTCTGAGG |
| Gag237 | HPVHAGPIA | CACCCTGTGCATGCTGGCCCTATTGCC |
| A10VV | APTKAKRRVV | GCACCCACTAAGGCCAAAAGGAGAGTGGTC |

Nucleic and amino acid sequences of pol2B7

AAGCCTGTGGTCTCCACACAGCTGCTTCTCAGGGCCAAGACCTTCCCTGT
GAGACCCCAAGTGCCACTGAGAAGGGCTGATACACAGCCCAGGAGTGACA
CCCATGTGTTCAGAACCAAGGCCATTCCTAGGAGAATTAGGCAGGGCCTG
AGAGATACAGCTACACCTCAGGACCTGAACACCATGCTGAGA
KPVVSTQLLLRAKTFPVRPQVPLRRADTQPRSDTHVFRTKAIPRRIRQGL
RDTATPQDLNTMLR

| | | |
|---|---|---|
| Name | aa seq | nuc seq |

| | | |
|---|---|---|
| K10LL | KPVVSTQLLL | AAGCCTGTGGTCTCCACACAGCTGCTTCTC |
| F10LR | FPVRPQVPLR | TTCCCTGTGAGACCCCAAGTGCCACTGAGA |
| Q9VF | QPRSDTHVF | CAGCCCAGGAGTGAdACCCATGTGTTC |
| I9GL | IPRRIRQGL | ATTCCTAGGAGAATTAGGCAGGGCCTG |
| T9ML | TPQDLNTML | ACACCTCAGGACCTGAACACCATGCTG |

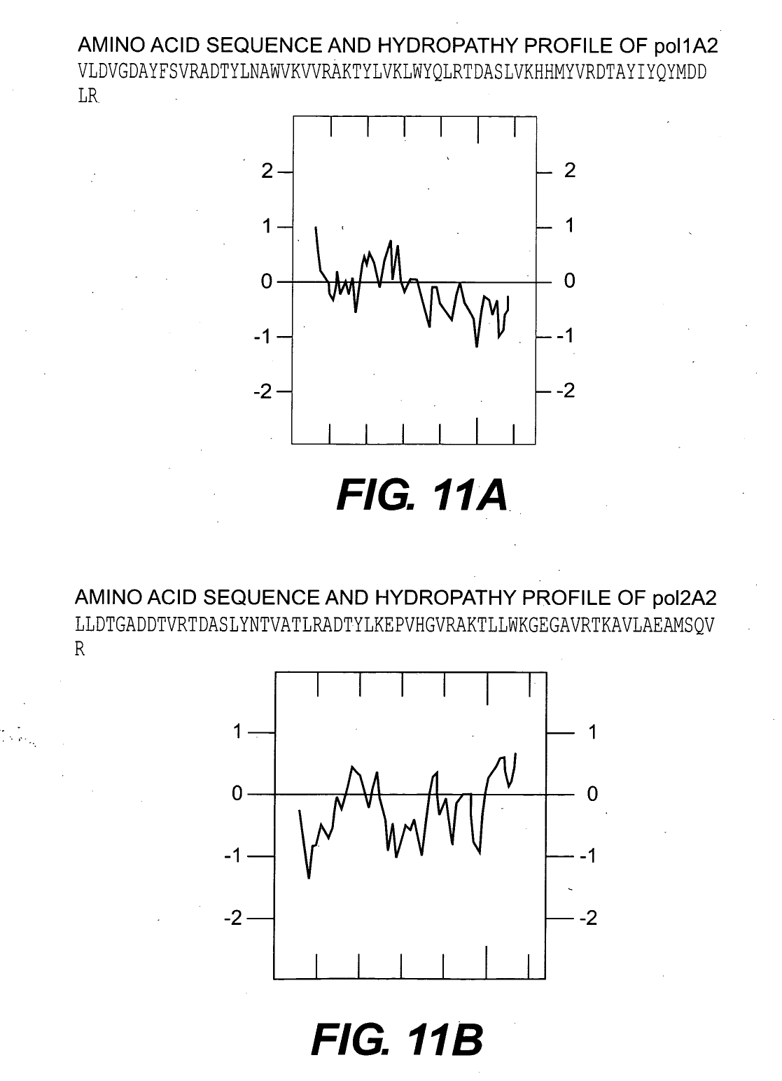

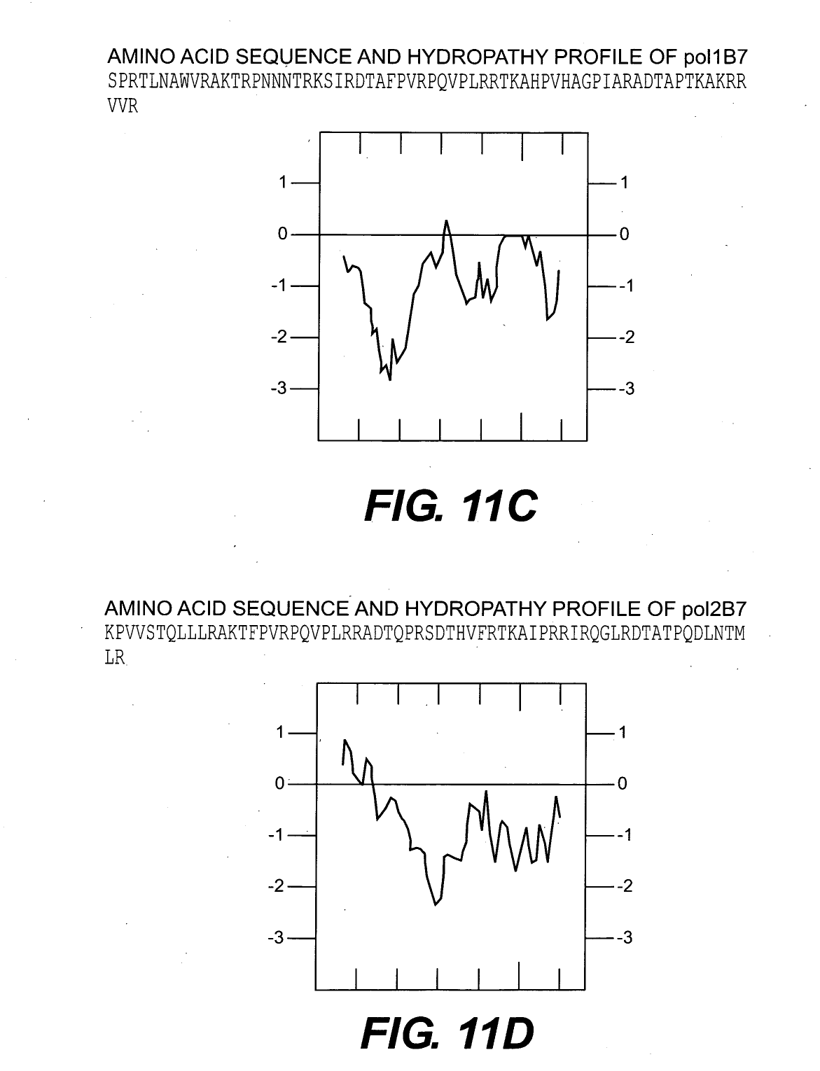

The amino acid sequences and hydropathy profiles of these HLA-A2.1- and HLA-B7-restricted HIV-1 epitopes are shown in FIGS. 11(A), 11(B), 11(C), and 11(D), respectively.

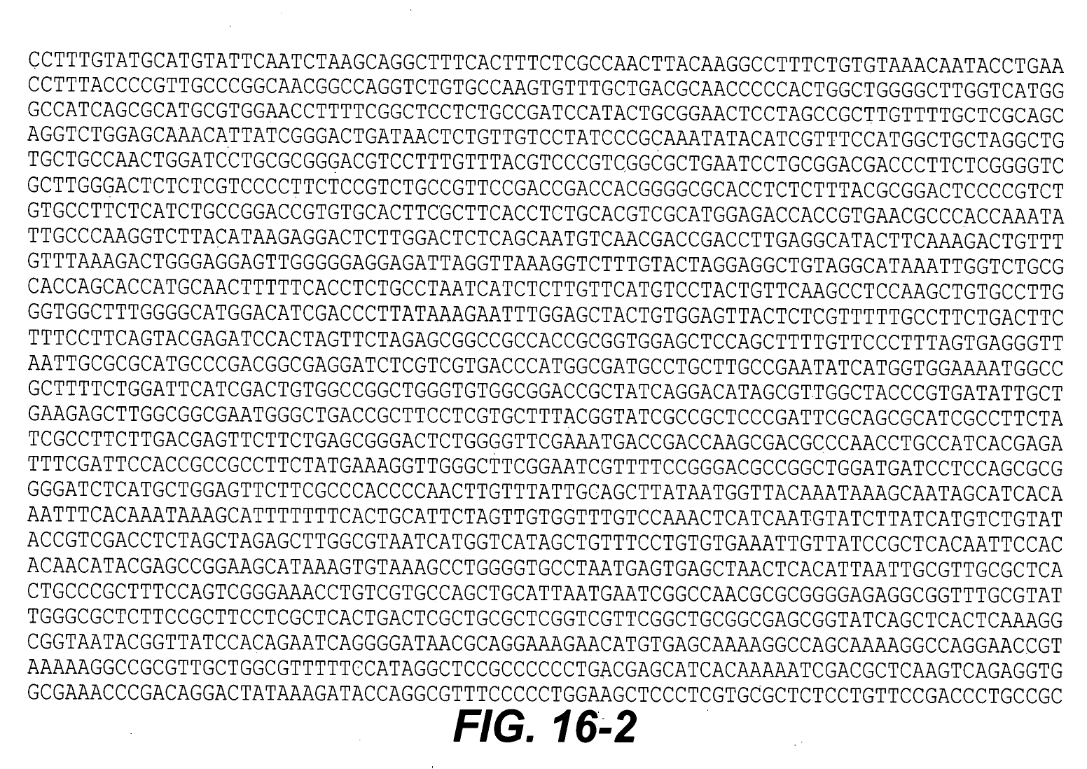

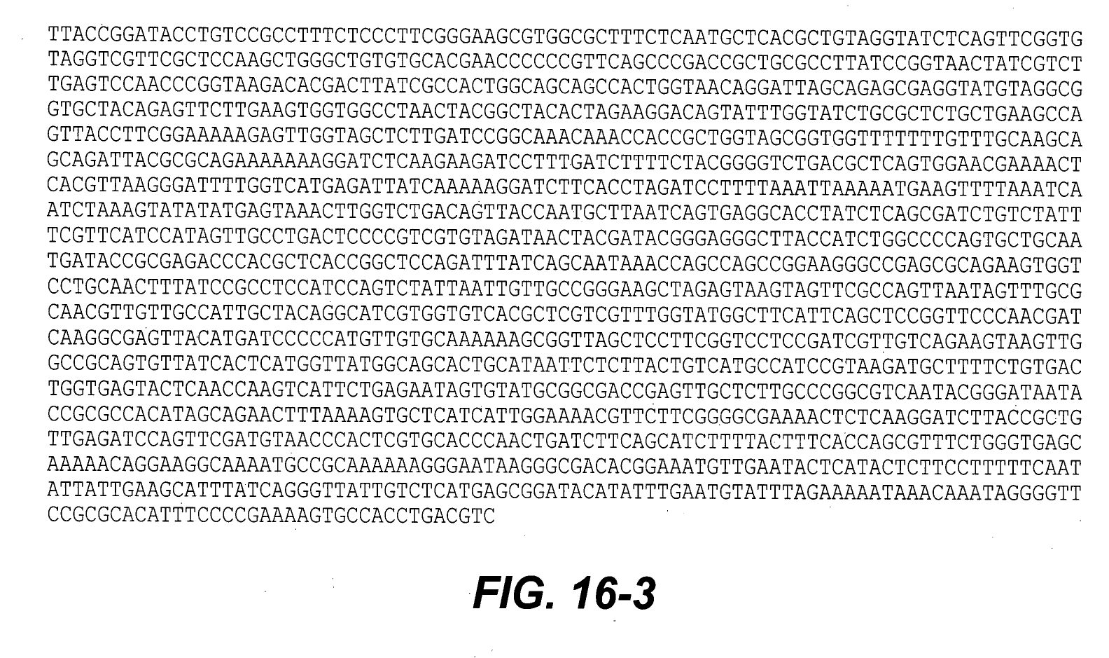

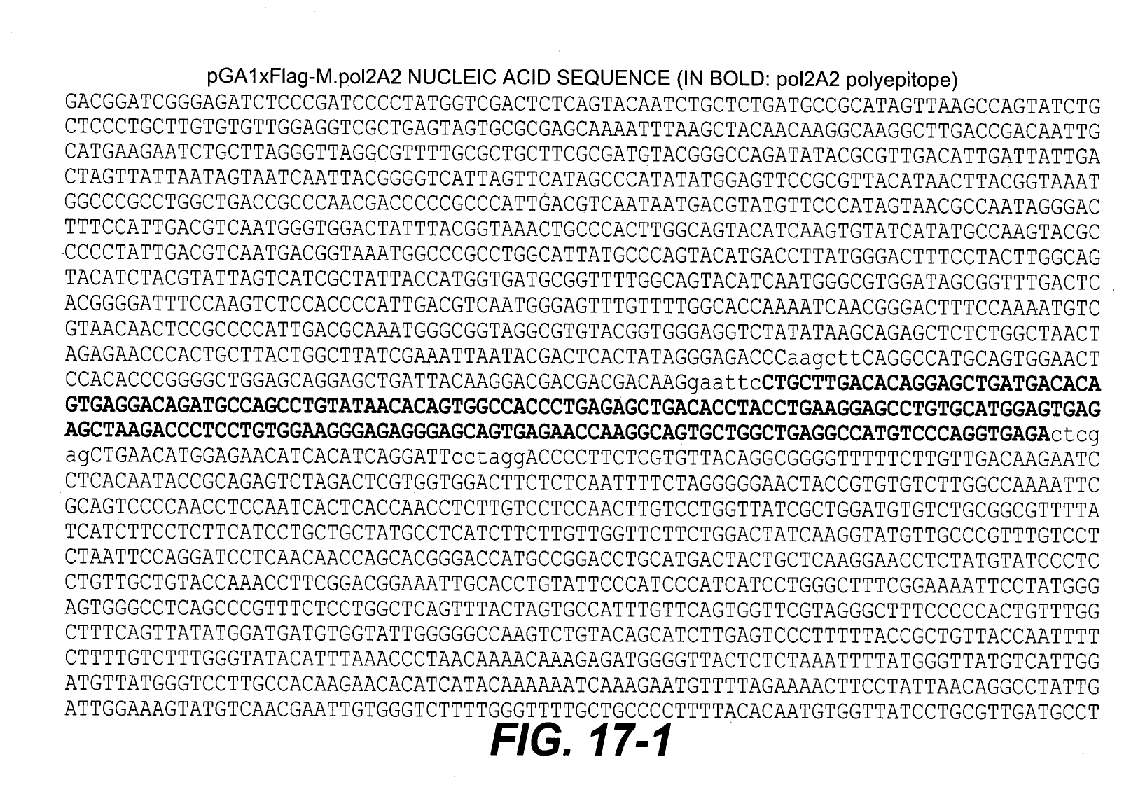

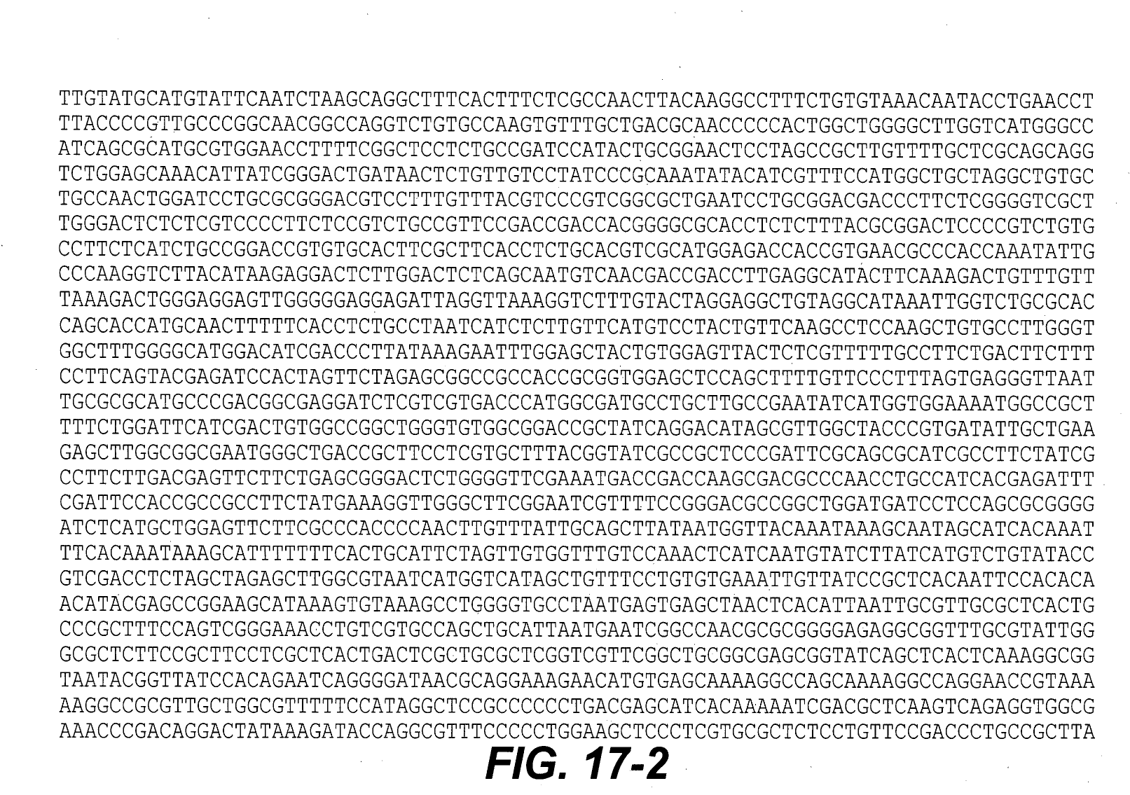

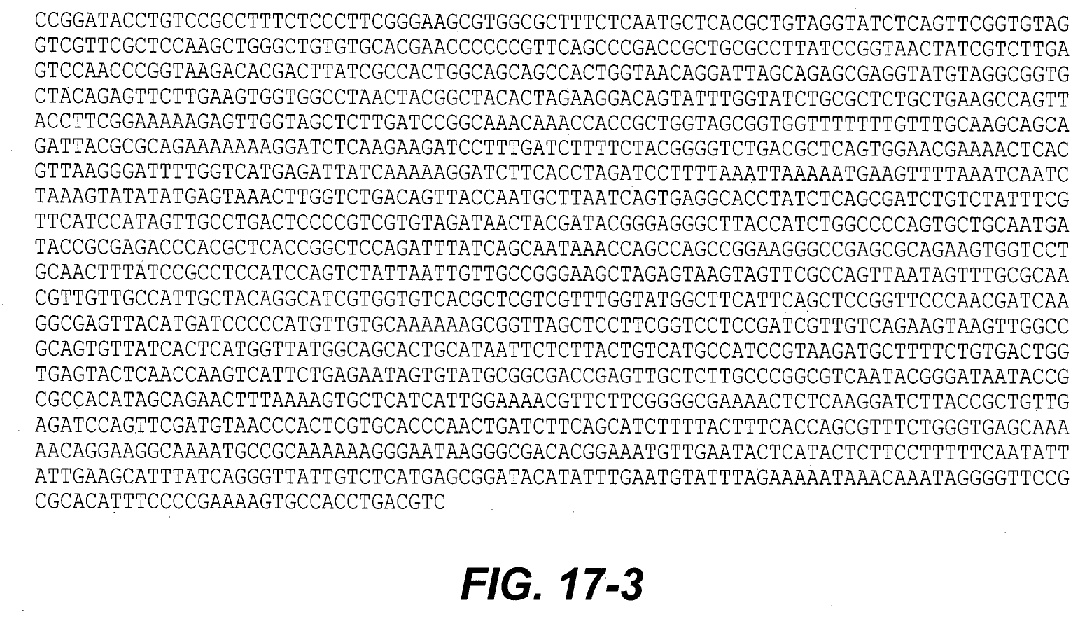

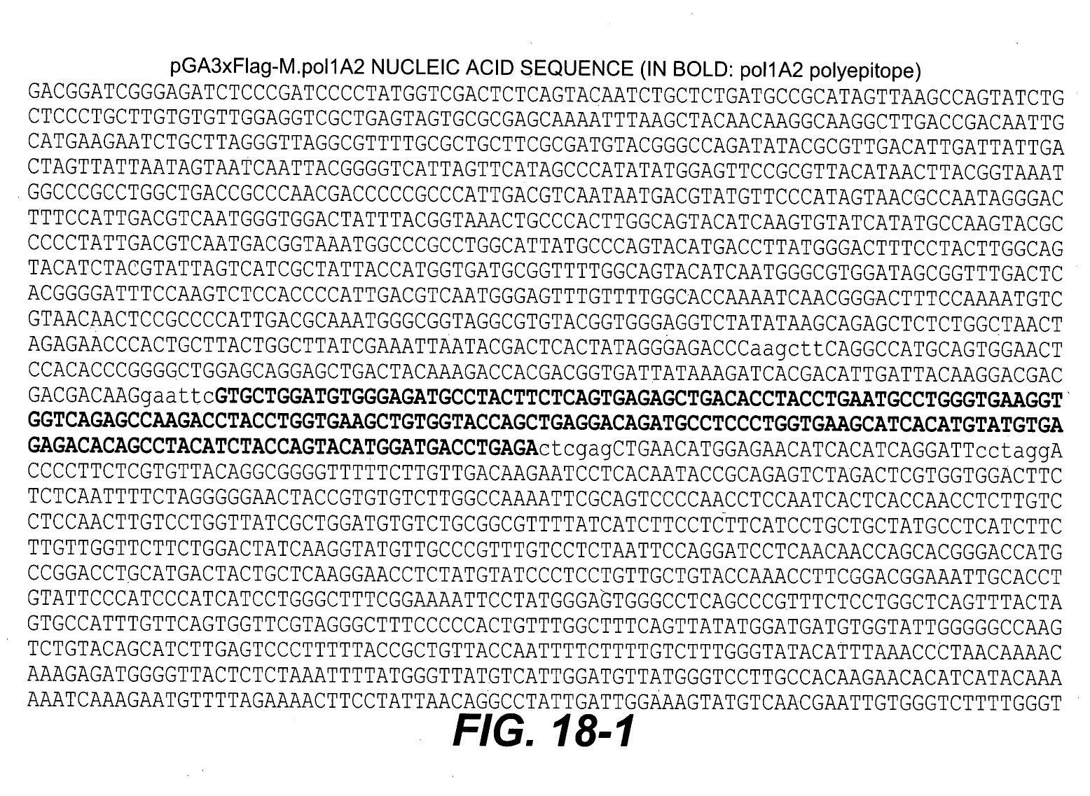

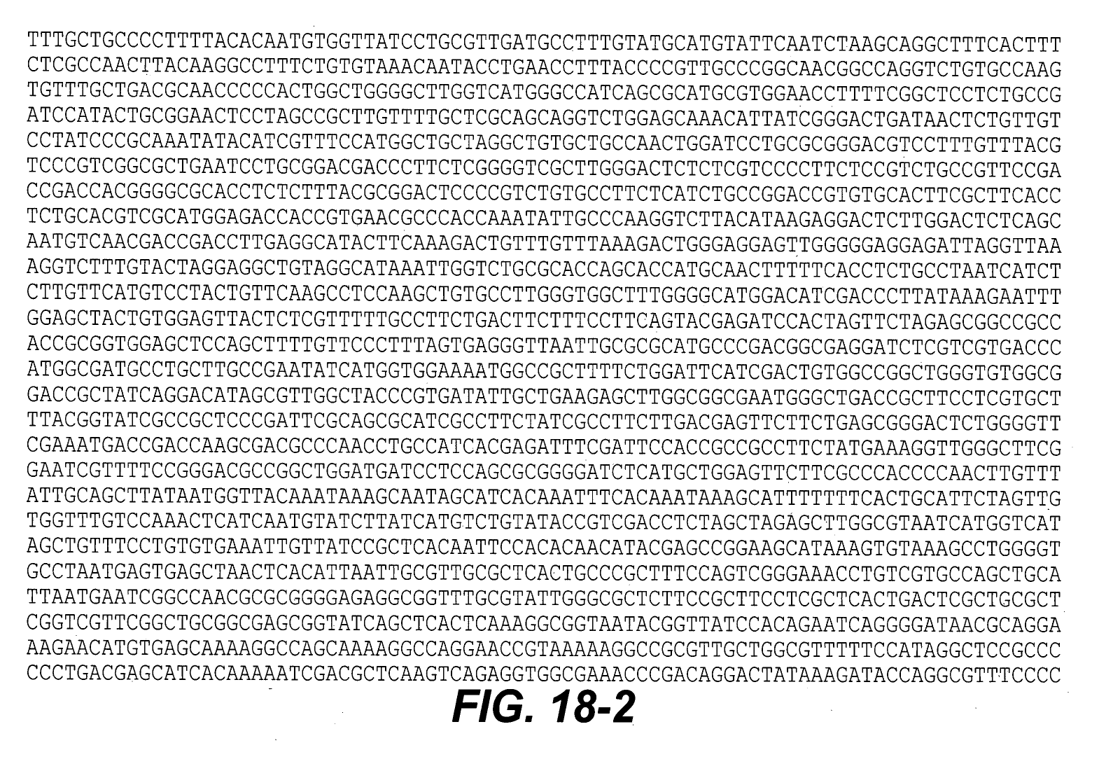

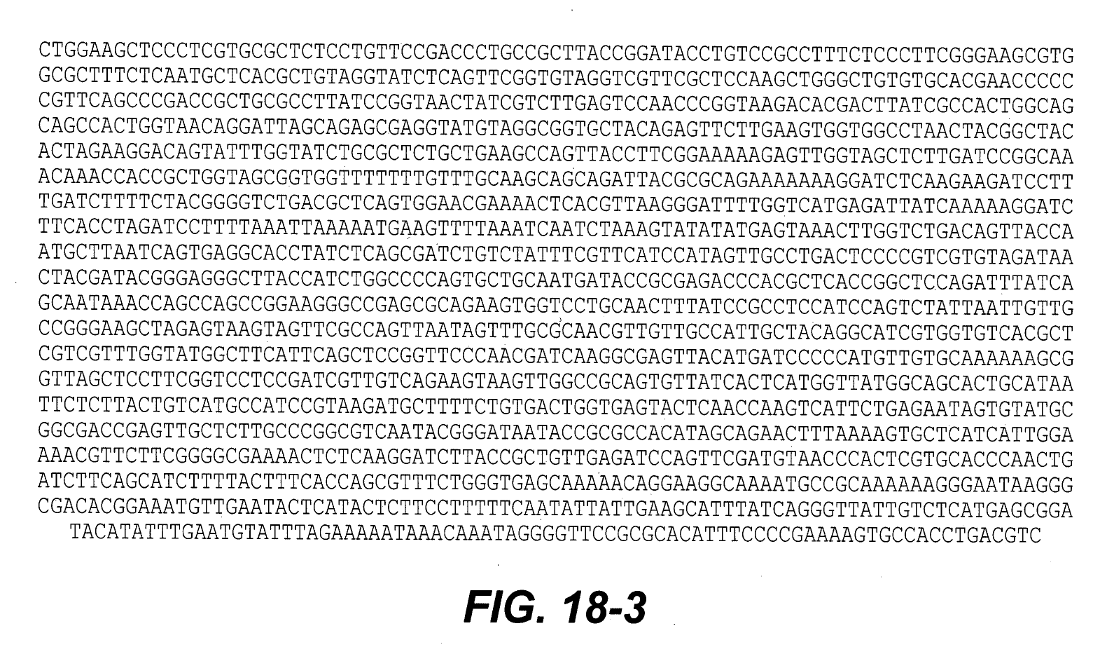

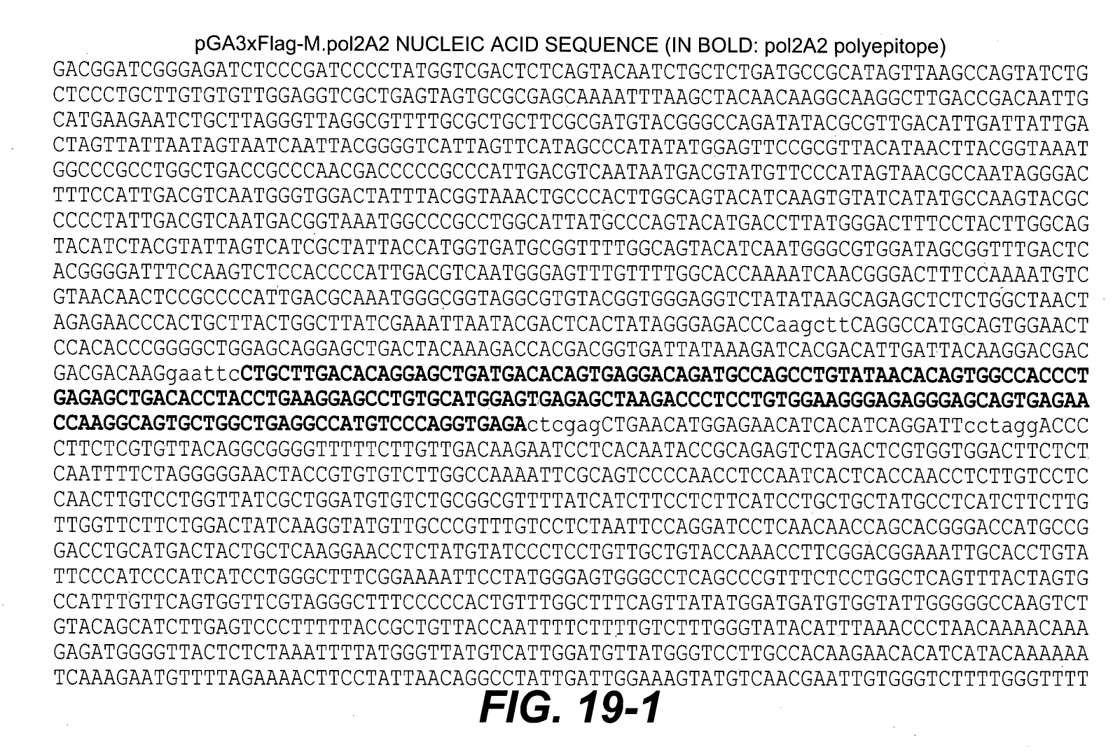

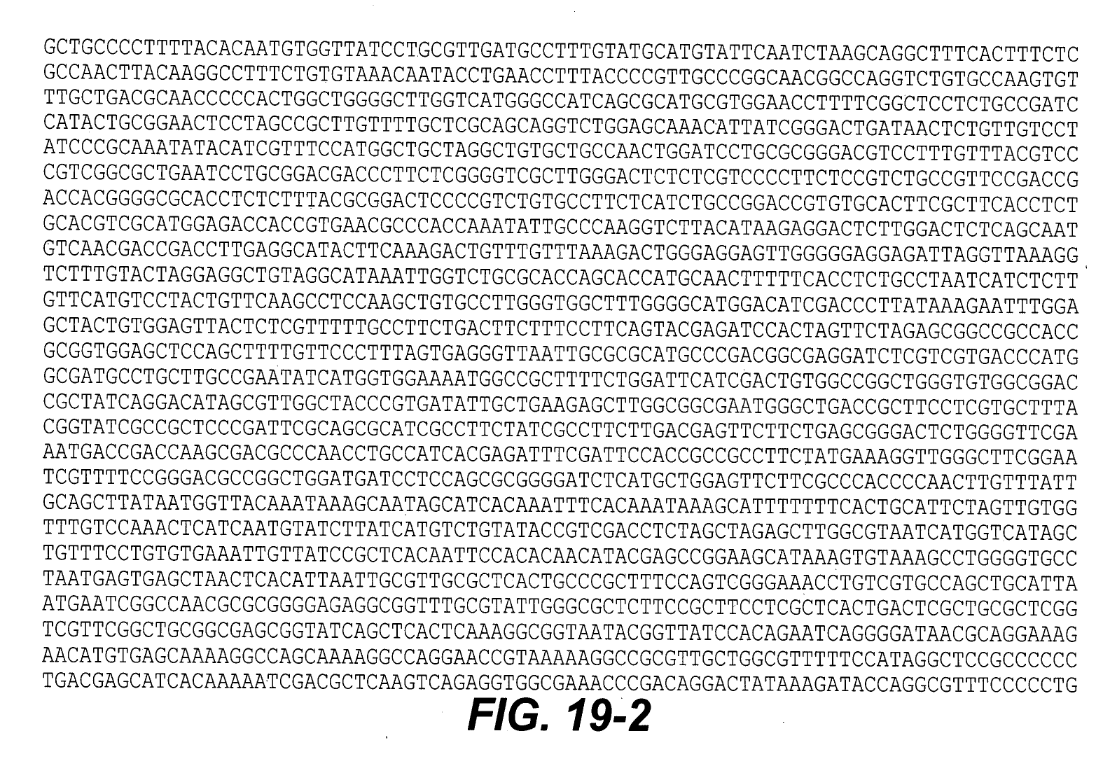

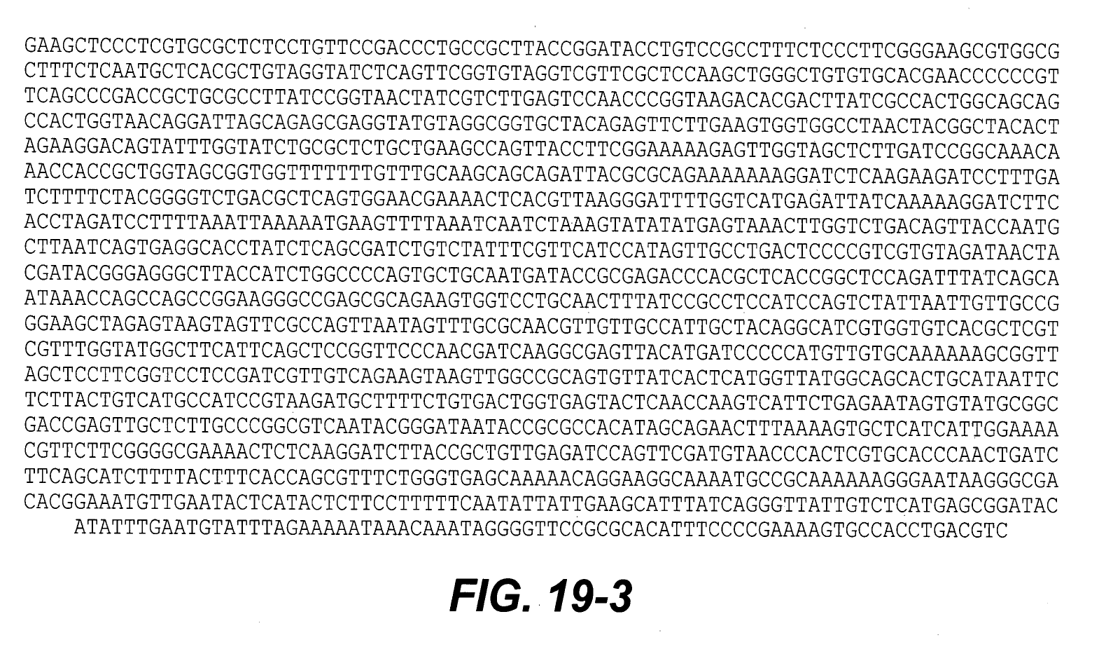

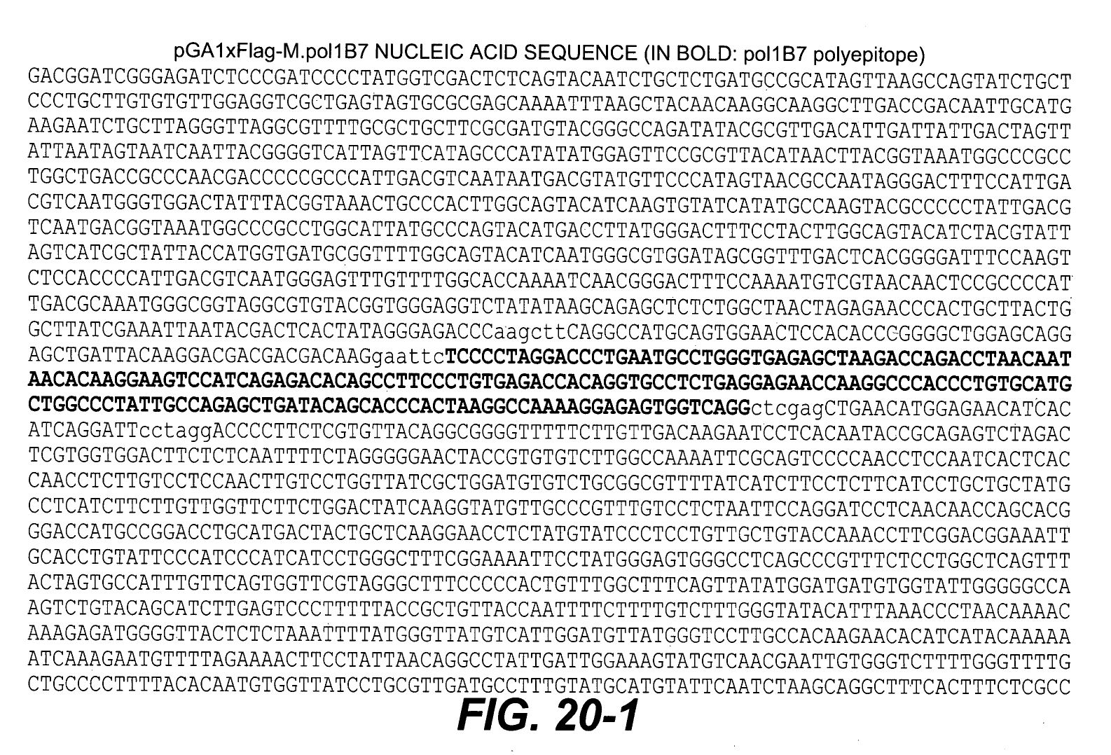

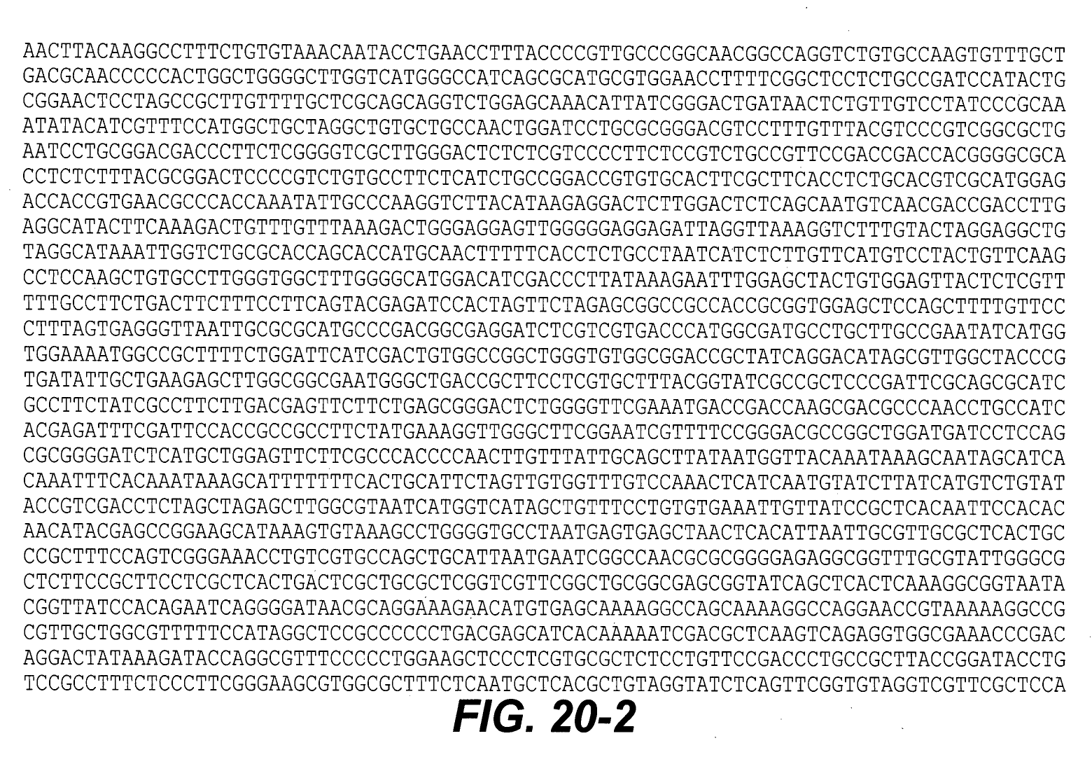

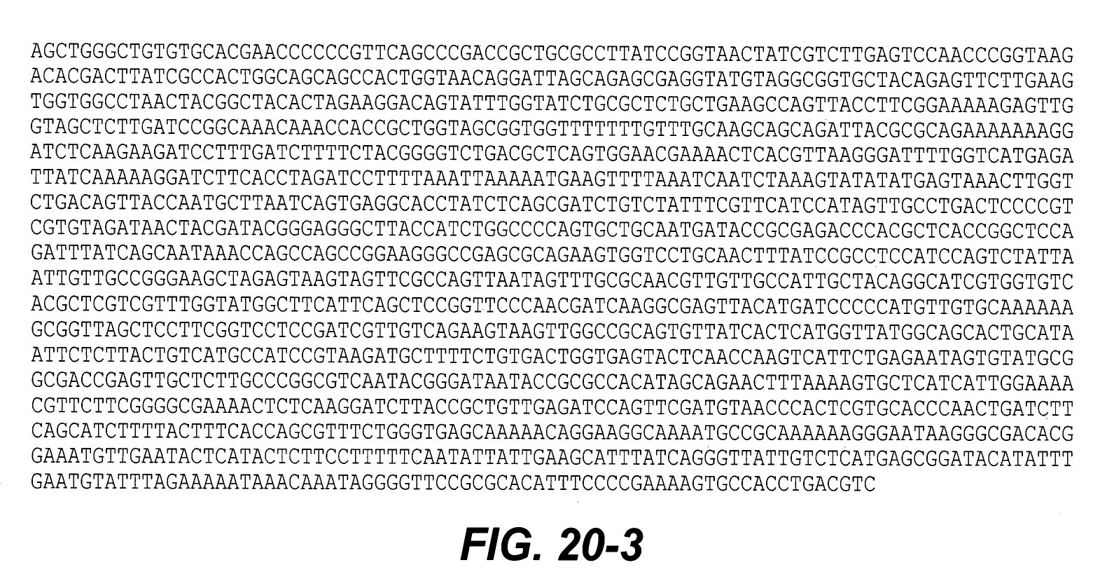

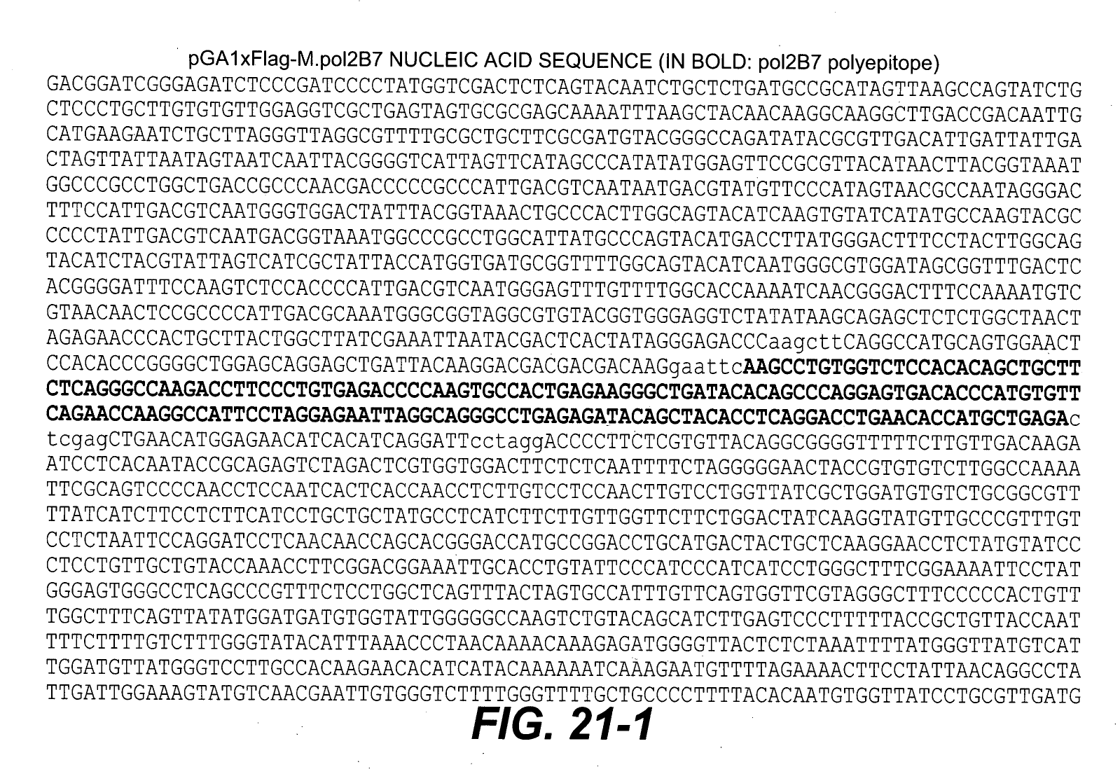

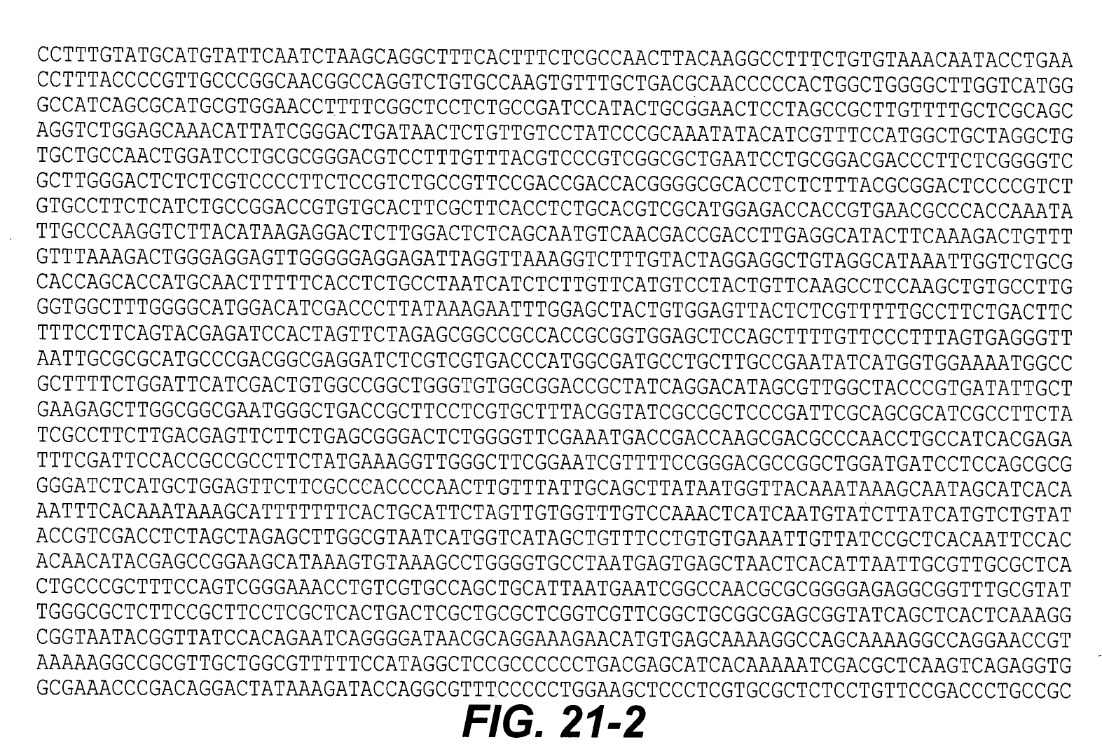

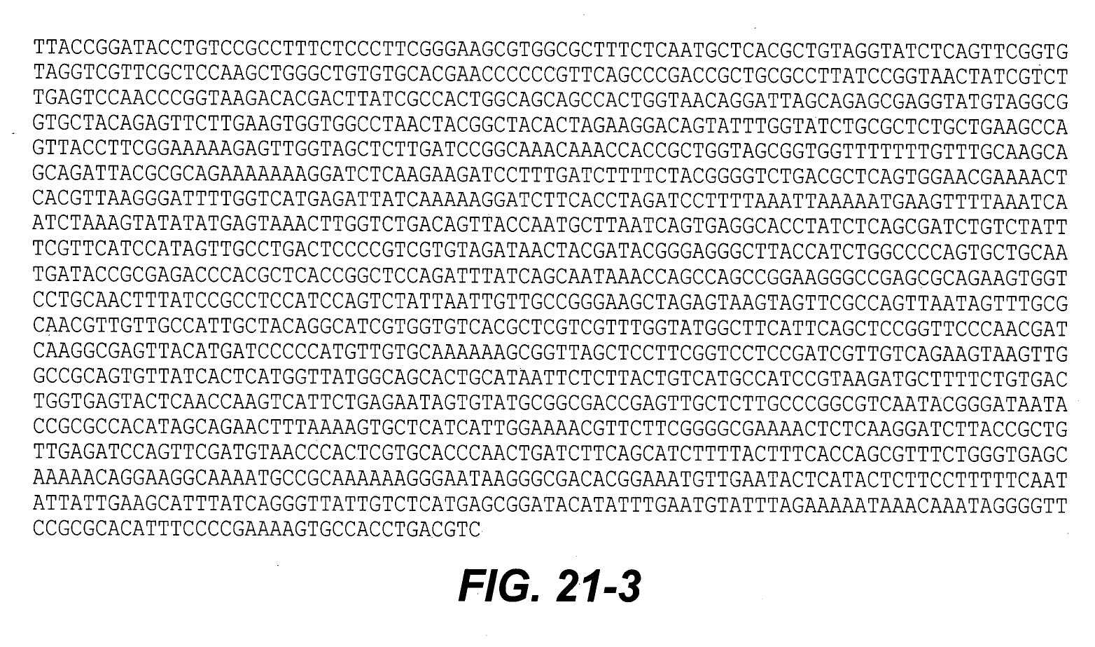

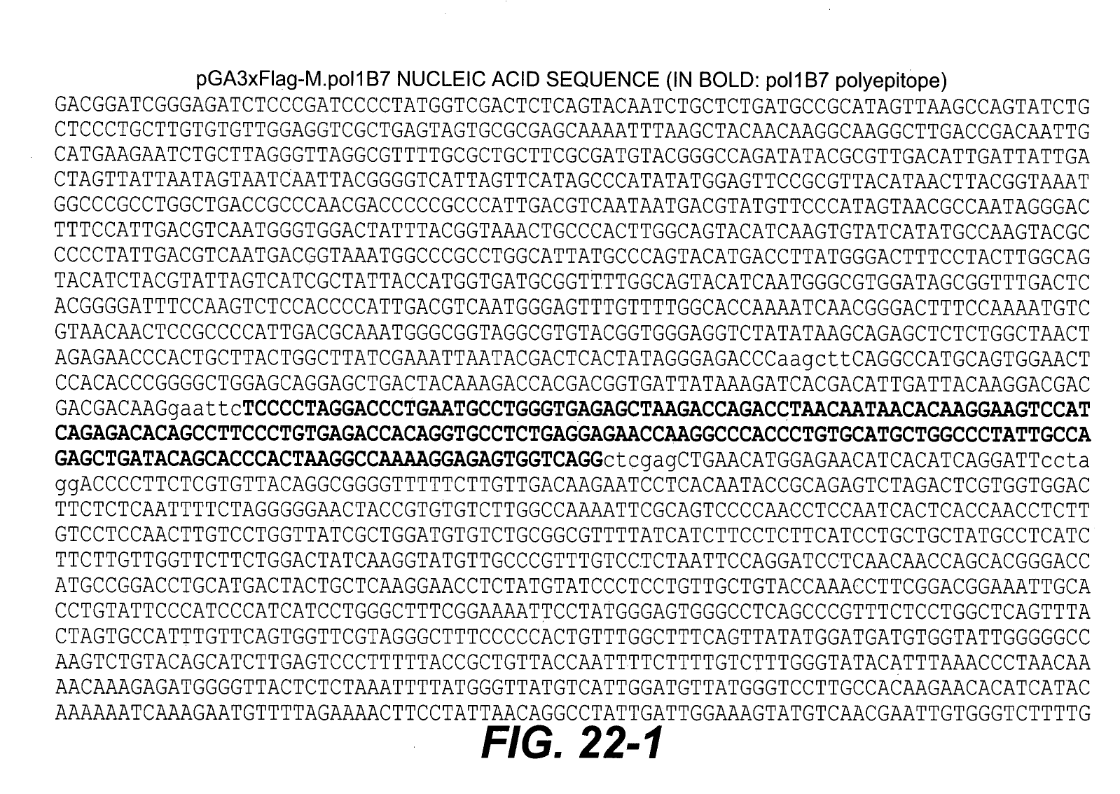

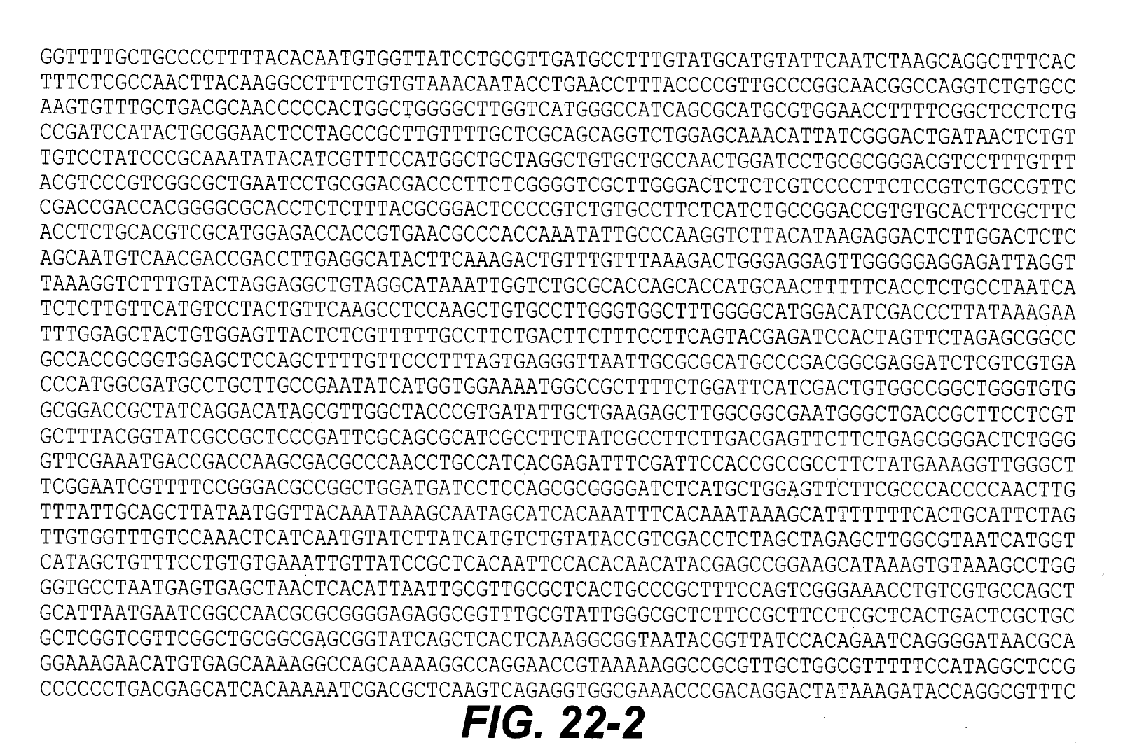

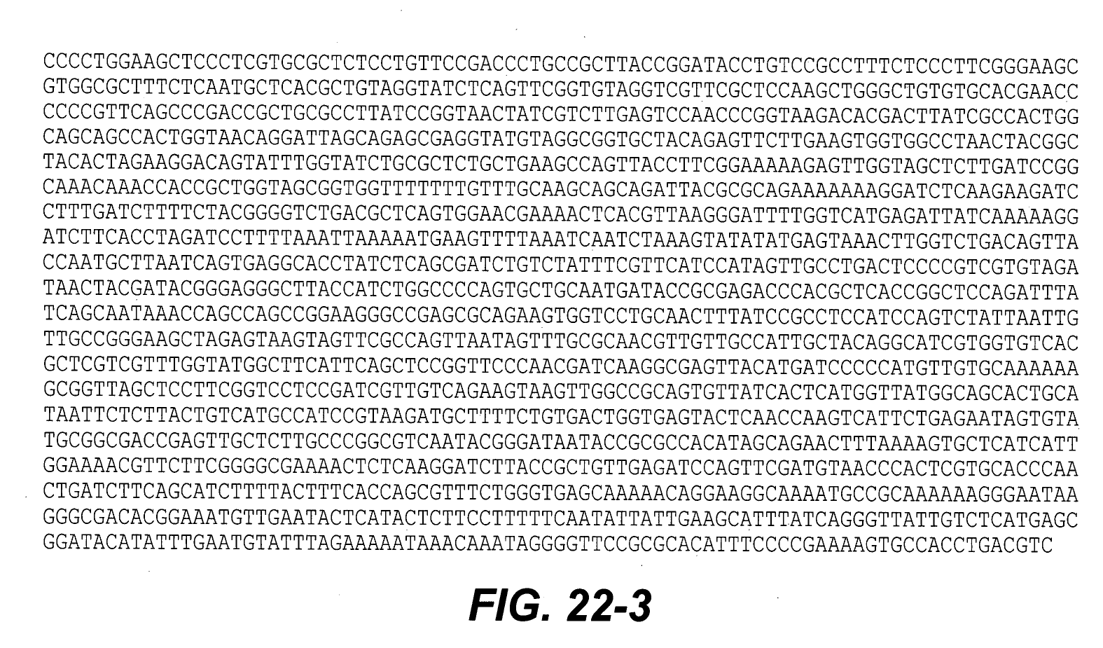

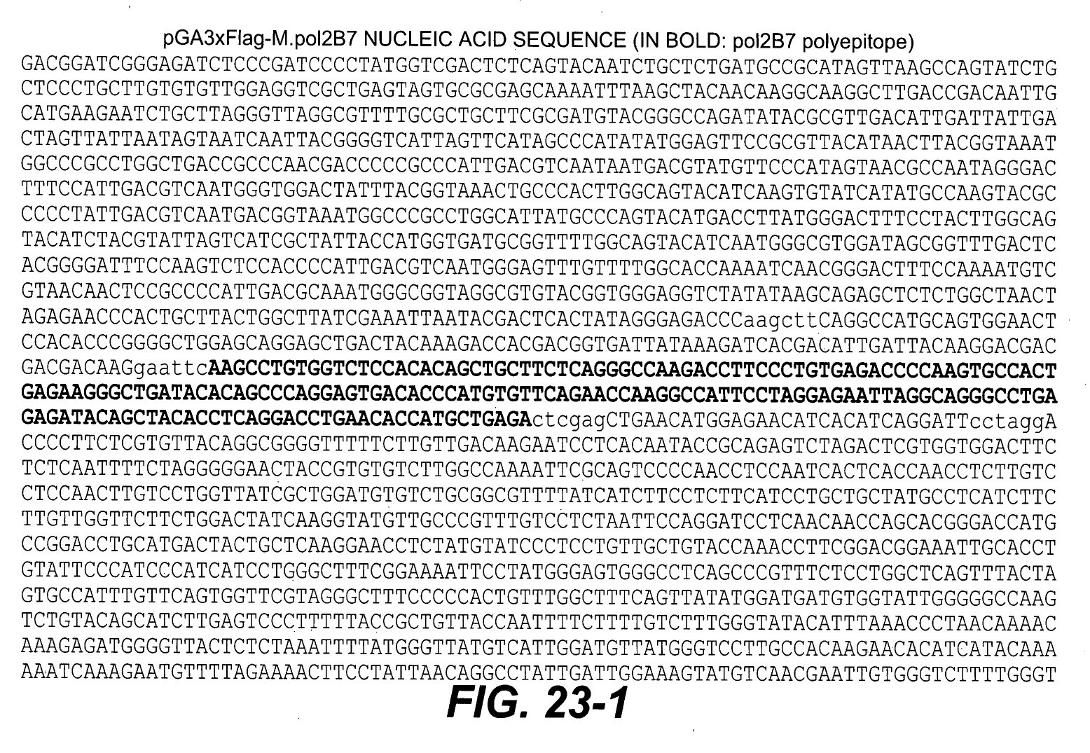

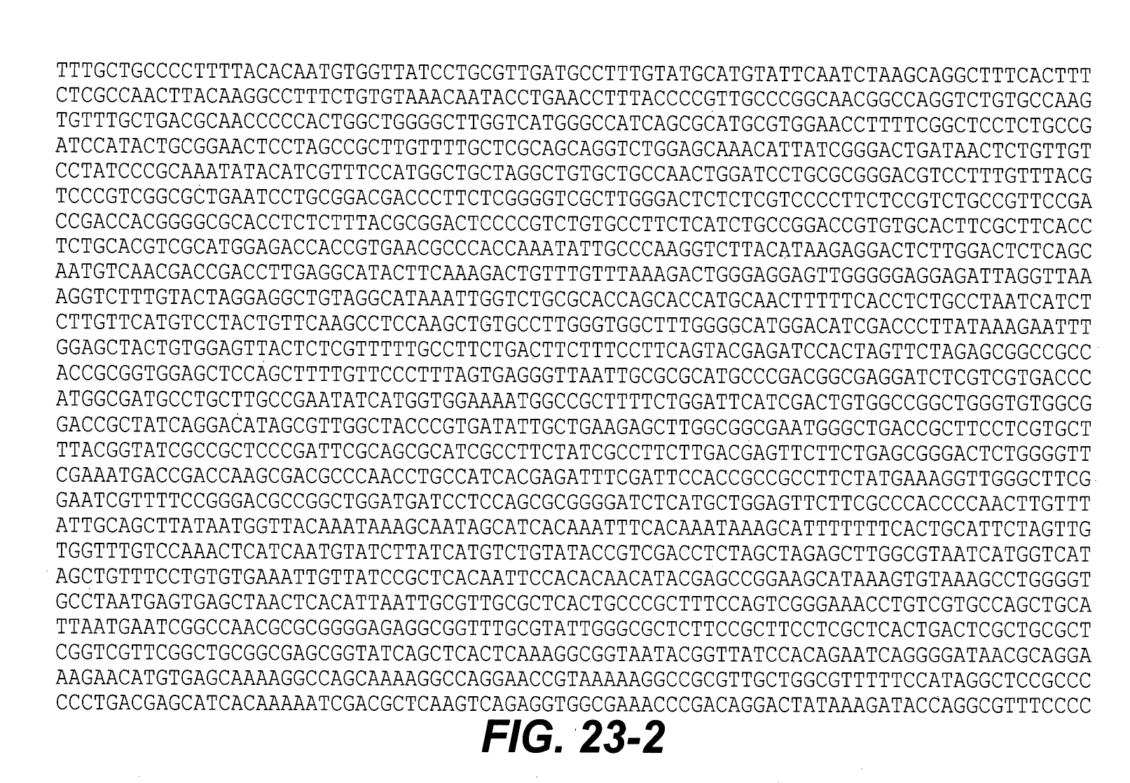

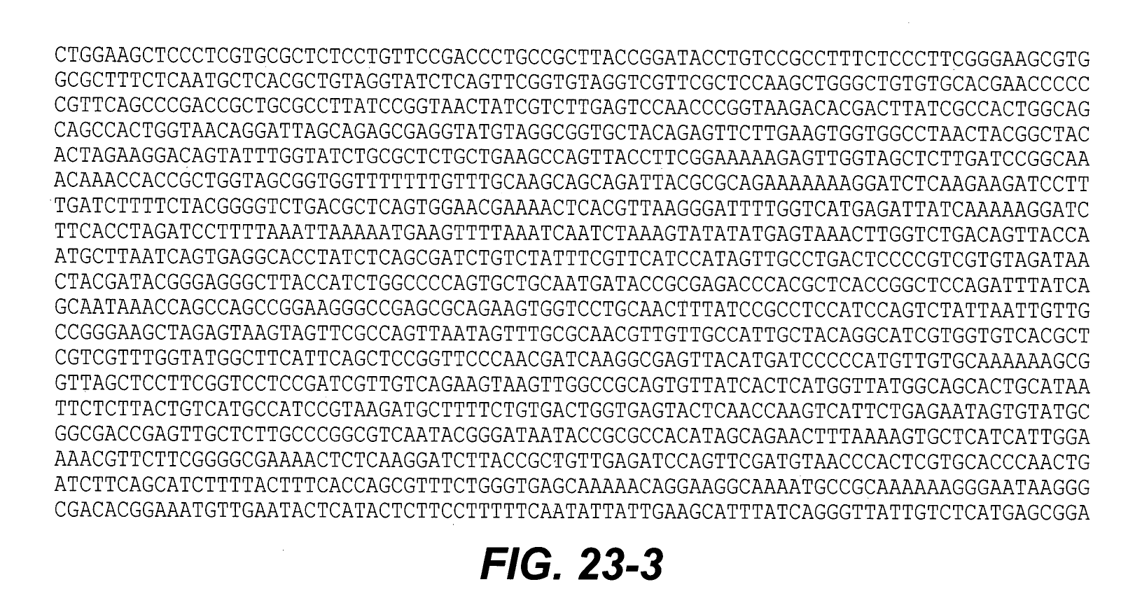

Each of the optimized polyepitopes pol1A2, pol2A2, pol1B7, and pol2B7 was similarly inserted into plasmid pGA1xFlag-M and pGA3xFlag-M. A detailed nucleic acid sequence for each of the resulting constructs is shown in FIGS. 16 to 23. The polyepitopic sequence inserted in the plasmid is shown in bold in each Figure.

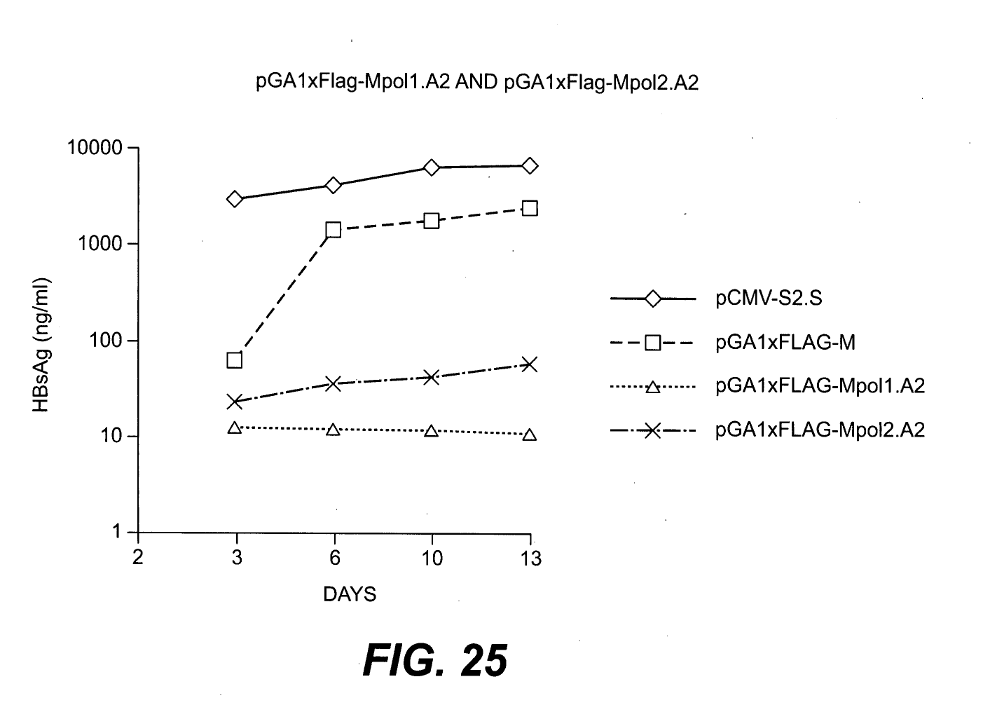

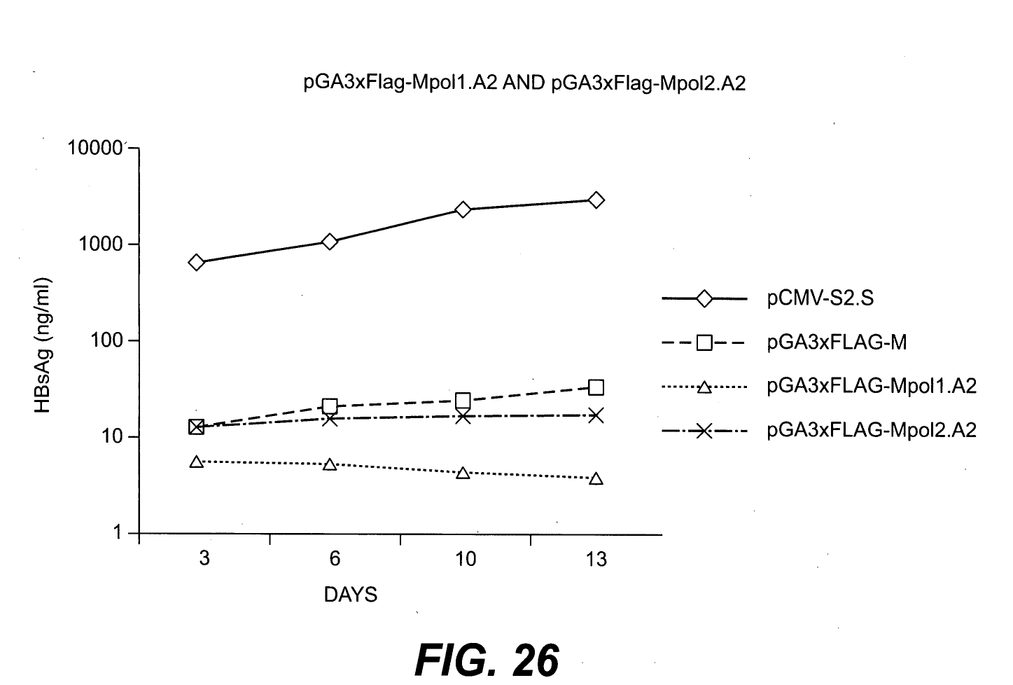

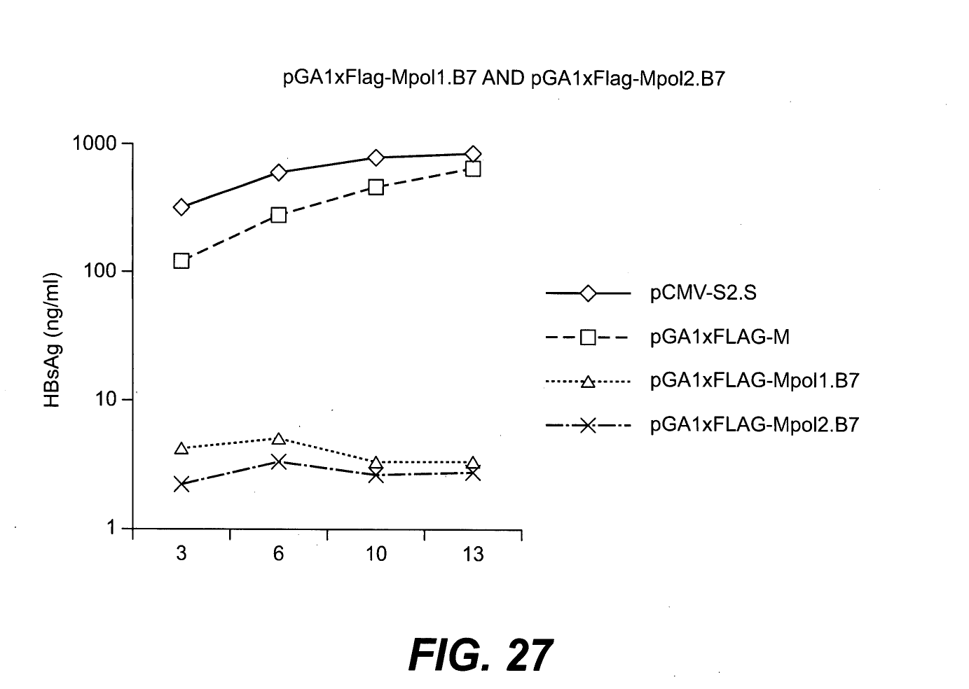

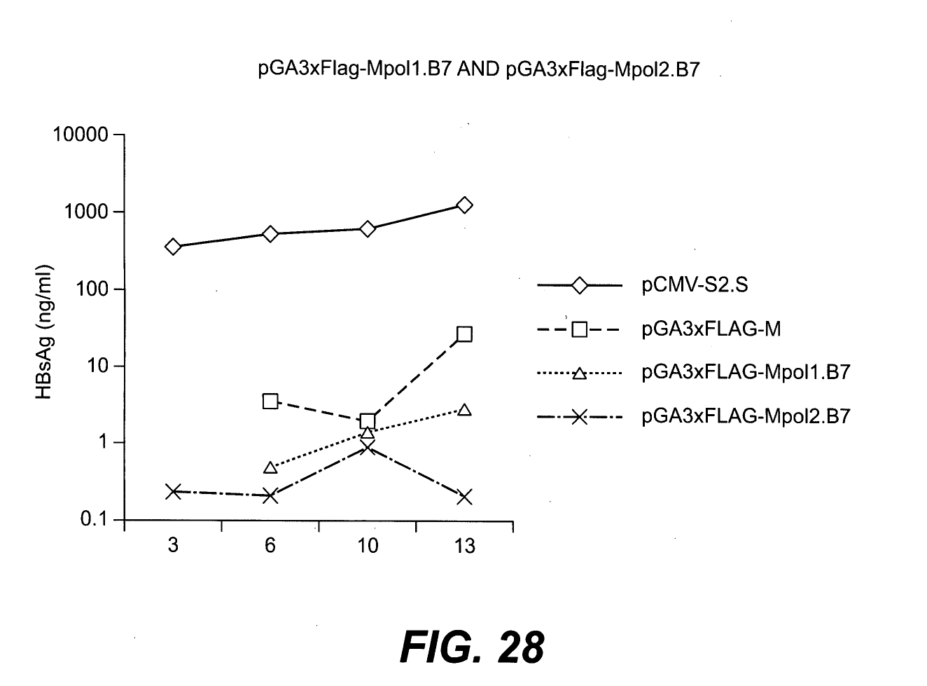

The recombinant HBsAg VLPs secretion kinetics corresponding to pGA1 xFlag-Mpol.opt, pGA3xFlag-Mpol.opt, pGA1 xFlag-M.pol1A2, pGA1xFlag-M.pol2A2, pGA3xFlag-M.pol1A2, pGA3xFlag-M.pol2A2, pGA1xFlag-M.pol1B7, pGA1xFlag-M.pol2B7, pGA3xFlag-M.pol1B7, and pGA3xFlag-M.pol2B7 transfections are shown in FIGS. 24 to 28. All constructions give rise to VLPs secretion from transfected cells. The lowest values are obtained by pol1B7 and pol2B7 bearing constructions. This is due to the fact that HLA-B7 restricted epitopes are more hydrophobic peptides, when compared to HLA-A2.1 restricted ones.

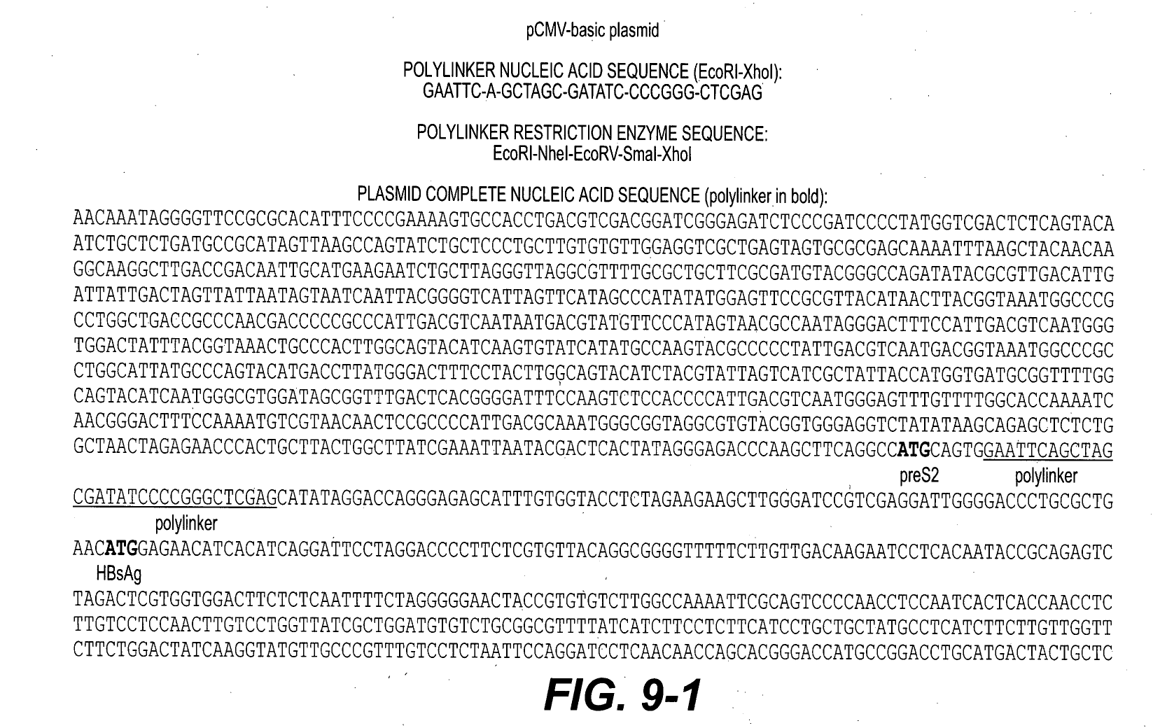

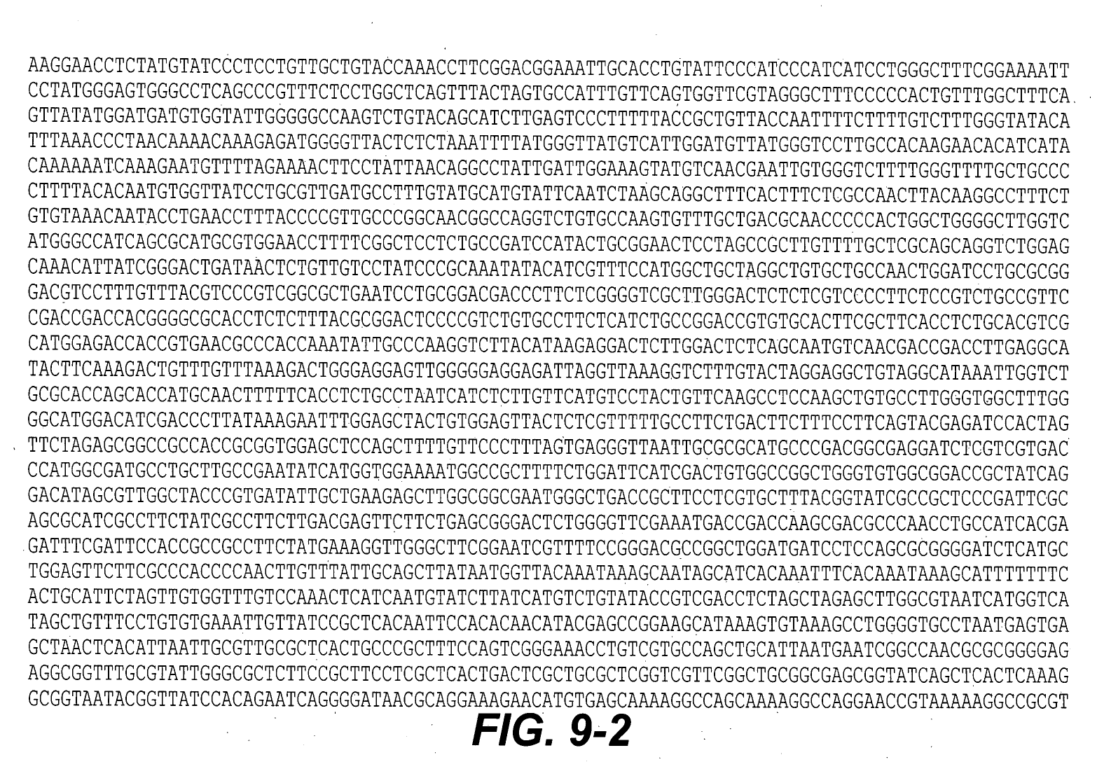

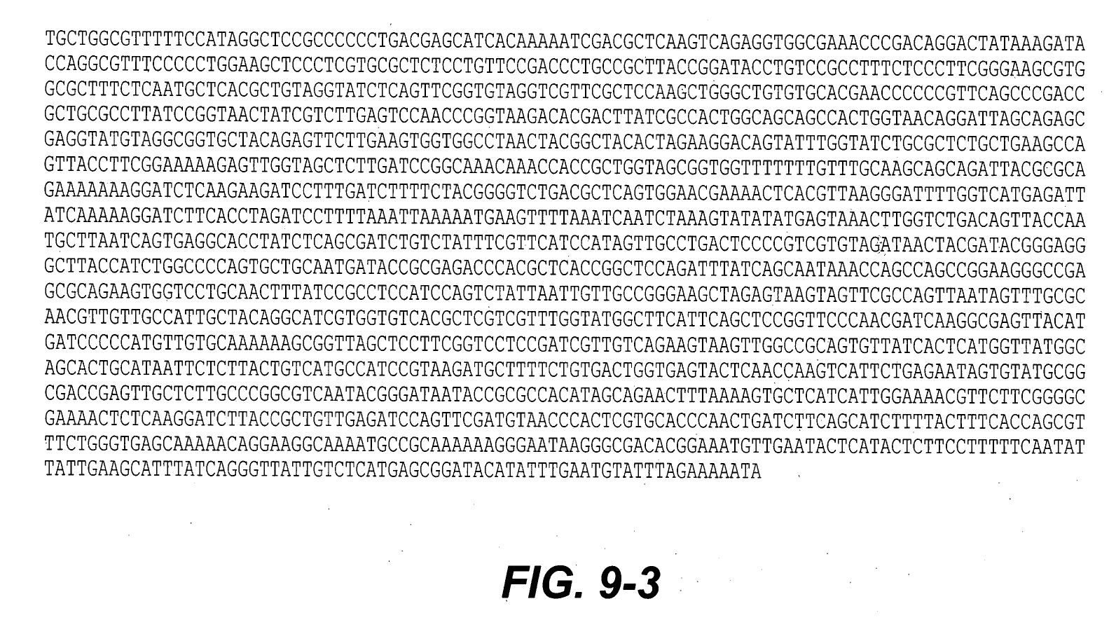

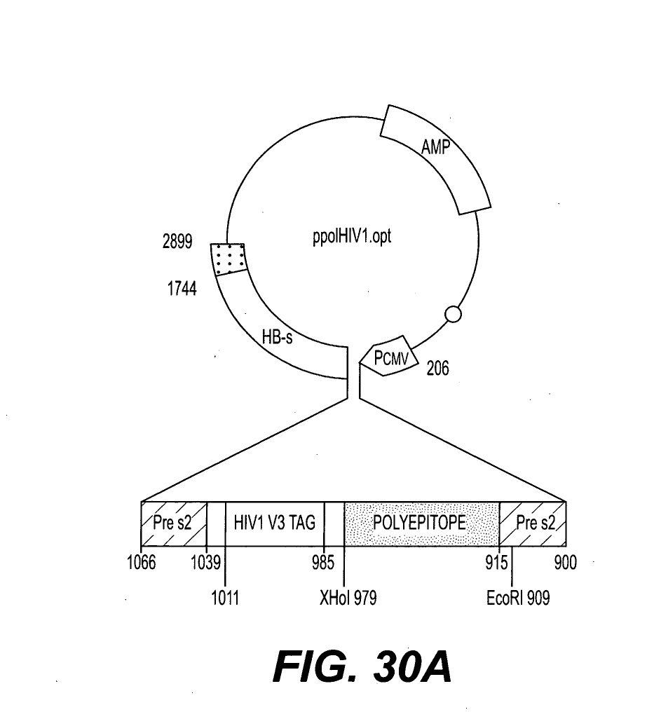

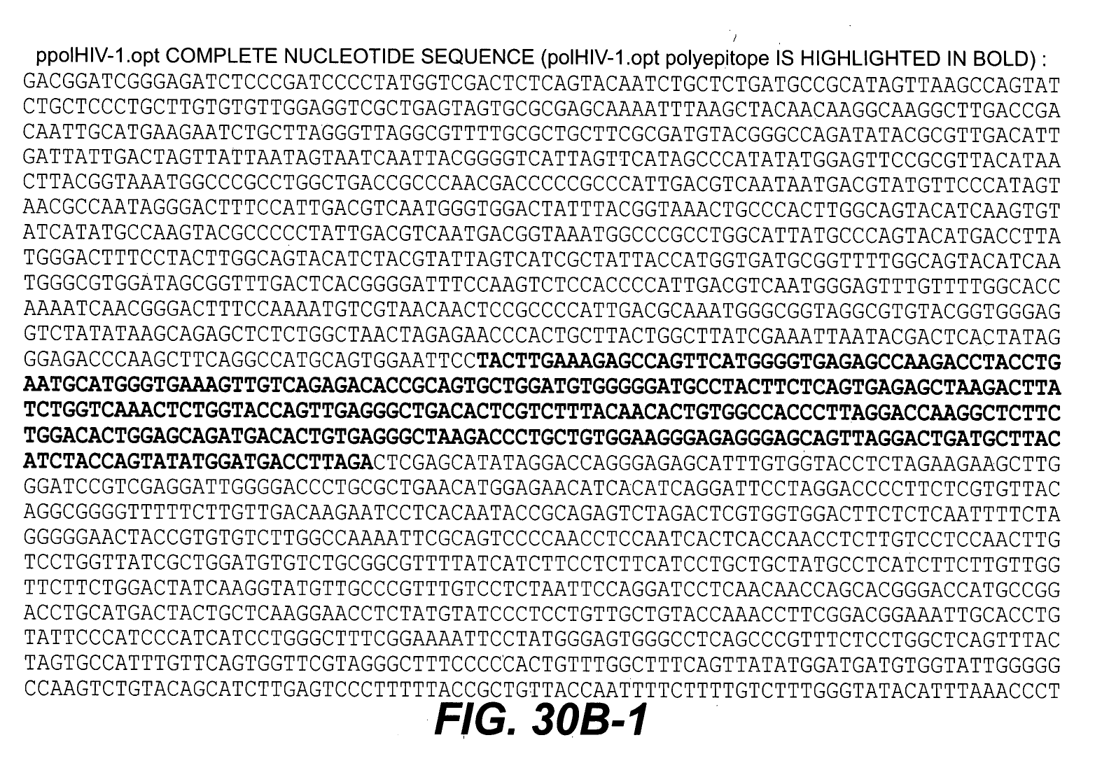

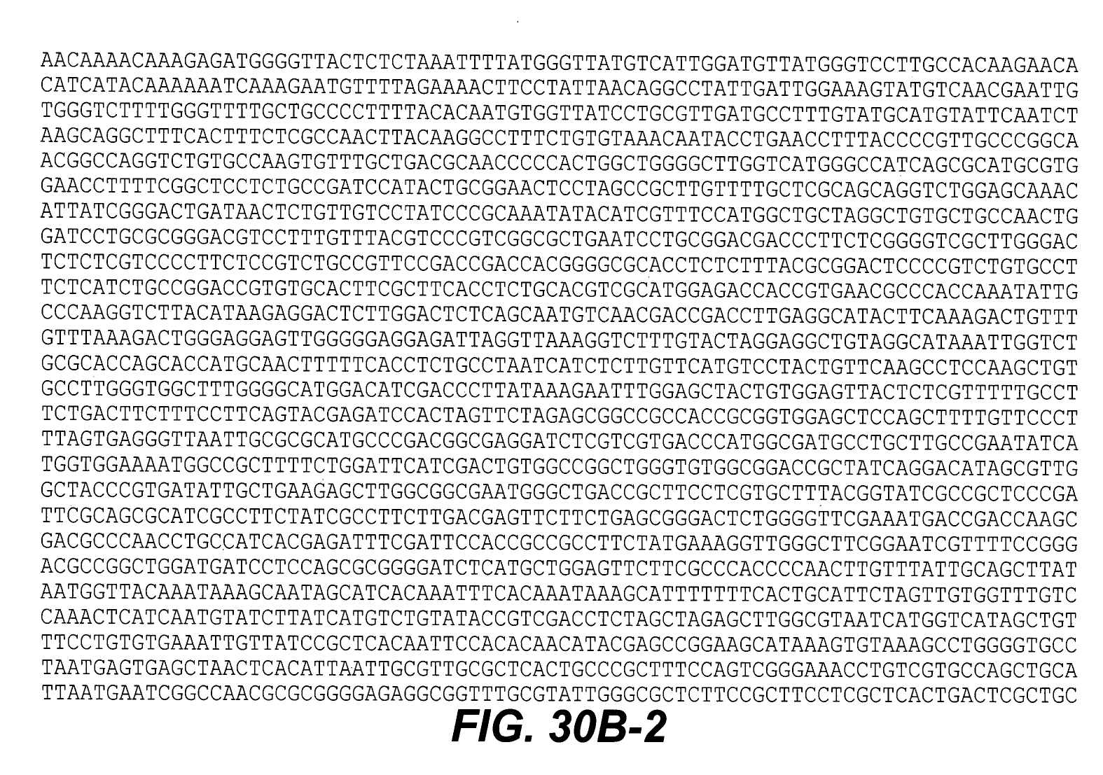

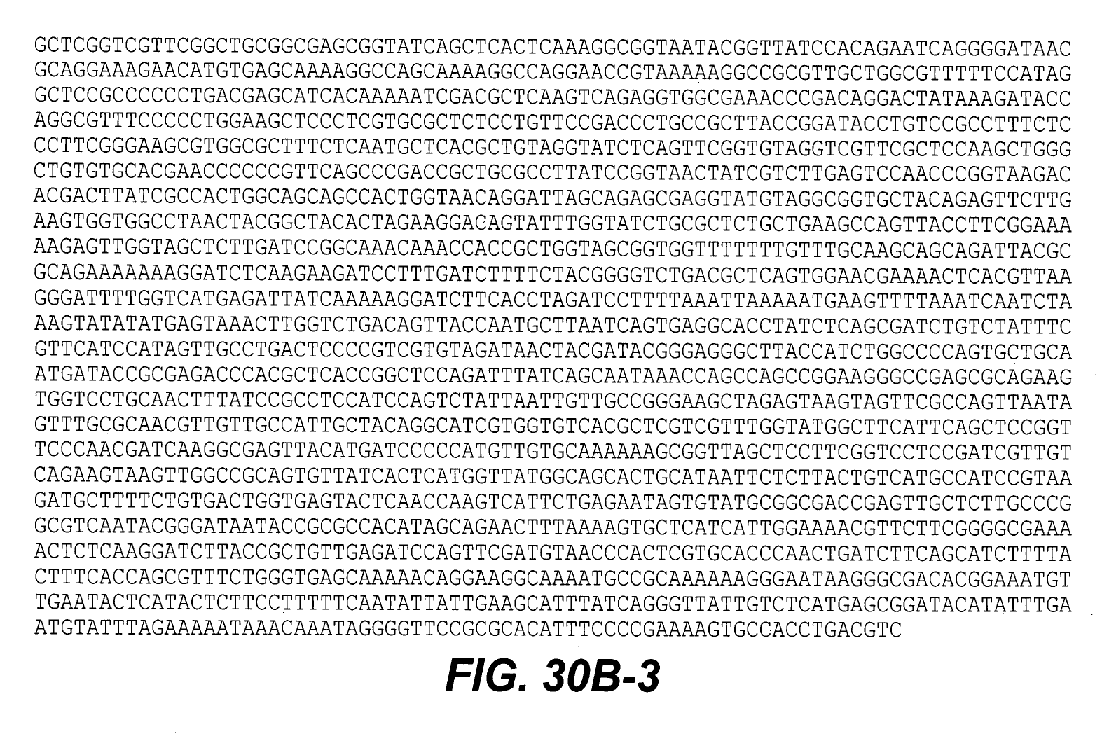

All in vitro analyses employed a control plasmid, the PCMV-basic plasmid (FIG. 9), which is derived from the ppolHIV-1.opt (FIG. 30). In this plasmid, the polHIV-1.opt polyepitope has been substituted by a polylinker where the EcoRI, NheI, EcoRV, SmaI, and XhoI restriction sites follow one the others (FIG. 9).

One embodiment of the invention based on the optimized polyepitope polHIV-1.opt will now be described in still greater detail.

Optimization of the polHIV-1 Polyepitope

An HIV-1 class I polyepitope composed of 13 HLA-A*0201-restricted minimal epitopes derived from different HIV-1 proteins had been engineered (polHIV-1; FIGS. 1A and 1B) and cloned into the preS2 region fused to HBsAg in the pCMV-B10 recombinant expression vector (16, 21), obtaining the ppolHIV-1 plasmid (12). Here, the preS2 and HBsAg ATG start codons preserve their relative strength at transcriptional level from HBV wild type nucleic acid contexts, the HBsAg one being the strongest. Hence, cloning into the preS2 region ensures the expression of two proteins from the same bicistronic mRNA (the polHIV-1/HBsAg recombinant and the HBsAg proteins), with greater production of the HBsAg protein.

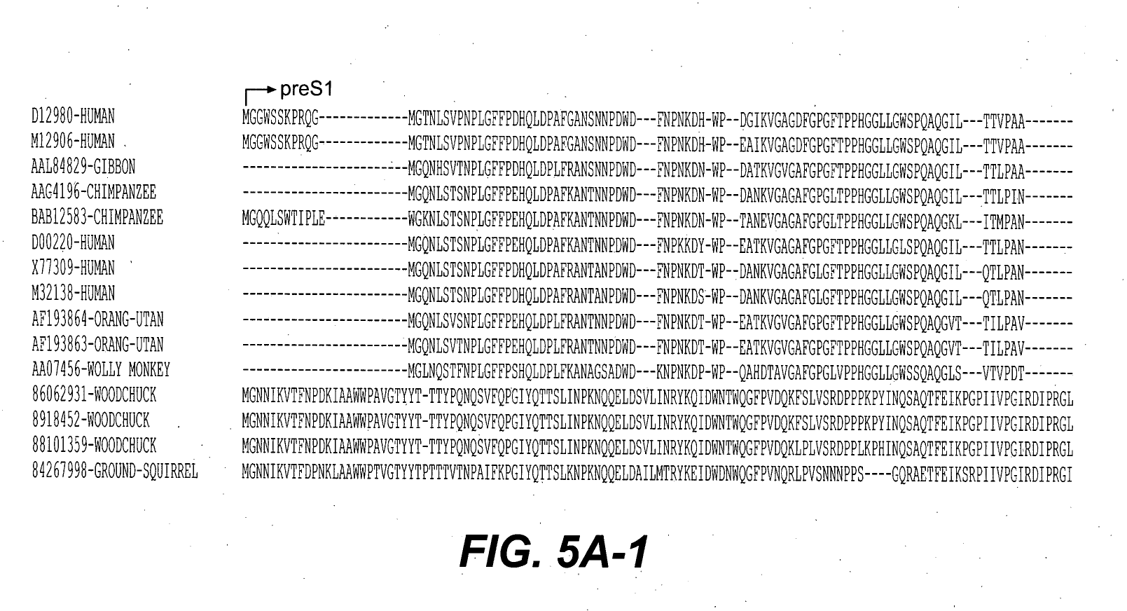

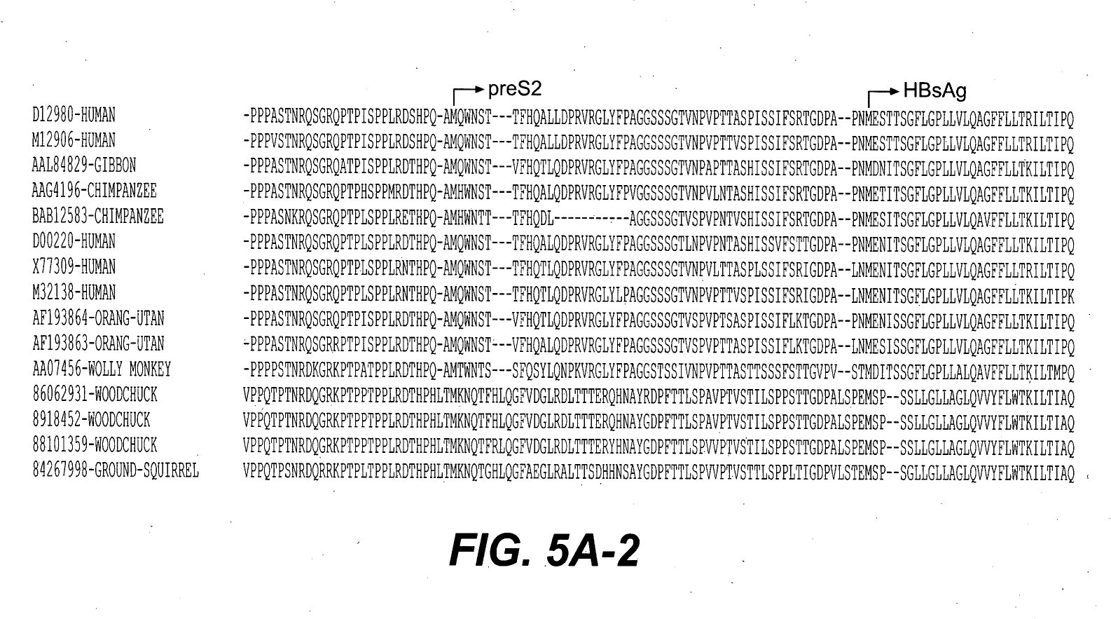

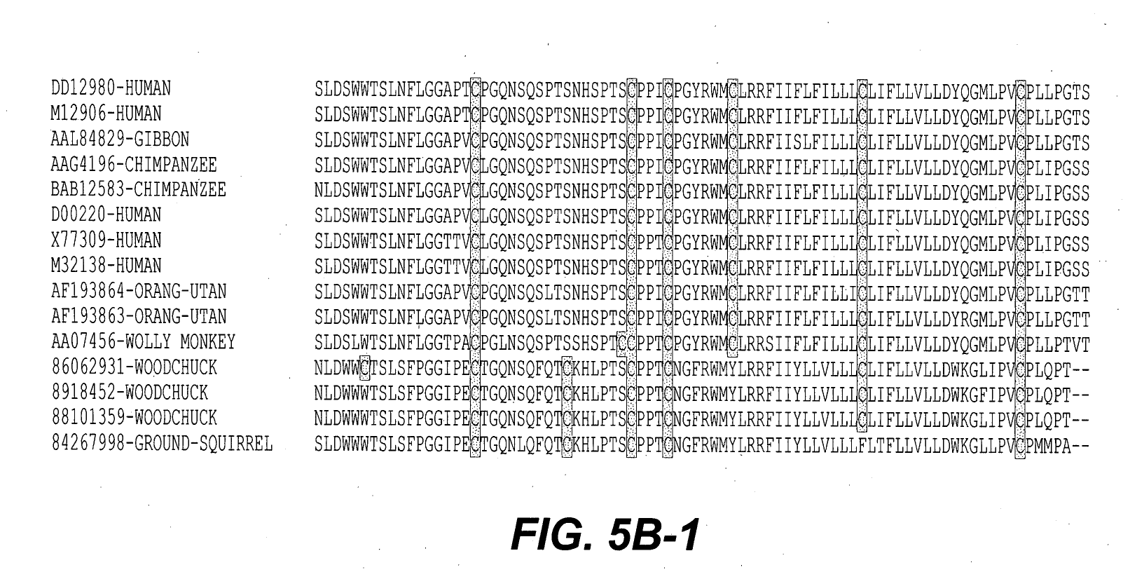

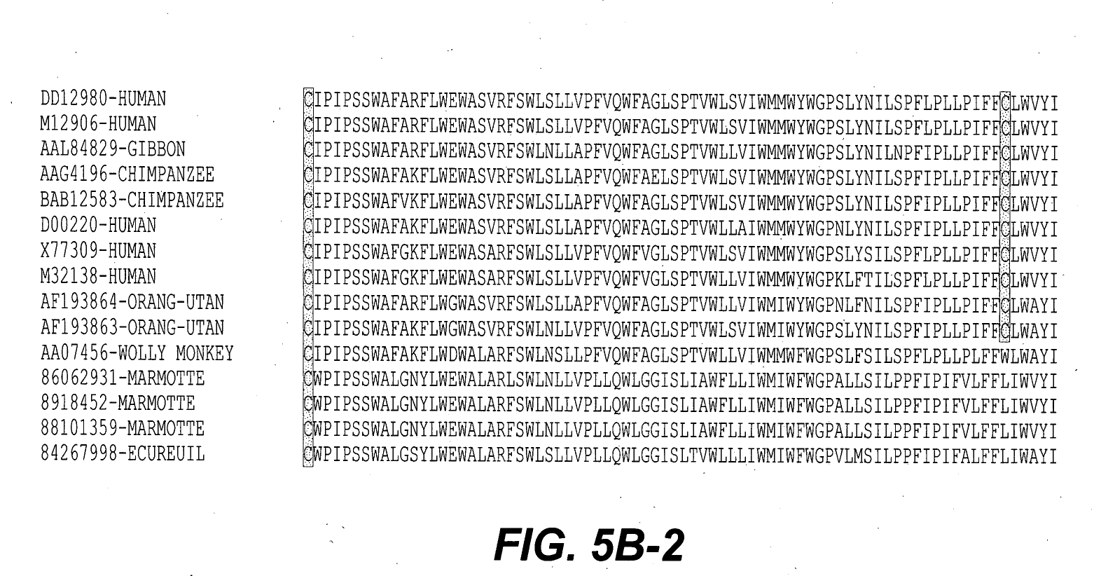

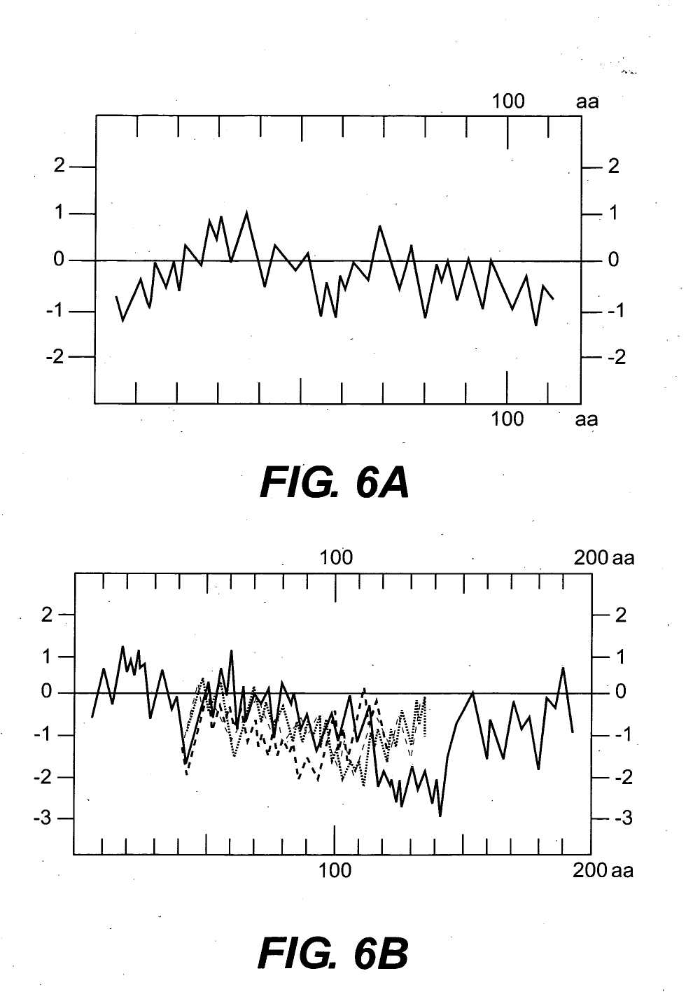

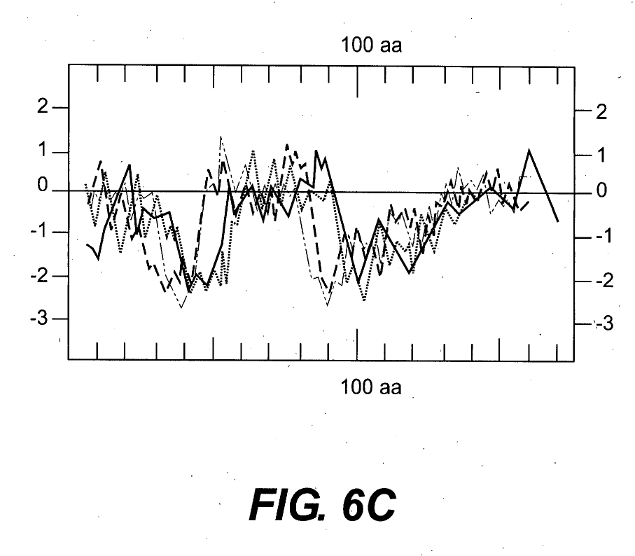

The comparison of both preS1/preS2 peptides from mammalian HBVs strains (FIG. 5: ftp://ftp.pasteur.fr/pub/retromol/Michel2006) showed that these regions are highly hydrophilic and are devoid of cysteine and methionine residues, apart from those necessary to initiate preS1 and preS2 translation. By contrast, the polHIV-1 polyepitope (FIG. 1B) was very hydrophobic (FIG. 1D), on a par with HBsAg itself, which spans the membrane four times. Furthermore, it presented five cysteines and four methionines. Mammalian HBsAgs encode fourteen cysteine residues (FIG. 5: ftp://ftp.pasteur.fr/pub/retromol/Michel2006), and it is possible that an additional five might disturb the correct formation of disulphide bridges. Of the four methionine ATG codons, three are of comparable strength to that of preS2 while a fourth is as strong as that for HBsAg itself and may indeed override it. Thus, the polHIV-1 could give rise to a series of proteins due to multiple initiation from methionine codons positioned downstream the preS2 ATG codon (FIG. 1A).

It was surmised that these features must be addressed in a redesigned polyepitope. Polyepitope optimization was sought at two levels, namely VLPs secretion and HIV-1 epitope processing. Accordingly, HIV-1 class I epitopes with cysteine residues were discarded. The single epitope (Y/V9L) that contains the well-known YMDD motif of reverse transcriptase and encodes a methionine residue was maintained in the optimized polyepitope (polHIV-1.opt; FIG. 1C). In the Y/V9L epitope, the ATG codon from its nucleic acid context would be no stronger than that of preS2. Hence, it was placed at the C-terminal region of the polHIV-1.opt polyepitope.

Class I epitopes are generally rather hydrophobic. To increase the overall hydrophilicity of the polHIV-1.opt polyepitope, small tetra-amino acid spacers were introduced in between epitopes. It has been demonstrated that the C₁-residue can influence class I epitope processing and exert a prominent effect on its immunogenicity (20). Indeed, higher levels of immunogenicity were correlated with the presence of basic, amide or small residues at the epitope C₁-terminus (20). Accordingly an arginine (R) residue was systematically placed in the epitope C₁-position. Four other amino acids were used, namely alanine (A), threonine (T), lysine (K) and aspartic acid (D), and the spacer sequence permutated. Permutation of residues which follow arginine was made to avoid at nucleic acid level repeated homologous sequence along the complete gene which could impair correct gene synthesis by using techniques based on polymerization (like PCR). At the amino acid level, the only aim is to increase hydropilicity, hence residues order is not important in itself. Furthermore, the choice of A, T, K and D is not exclusive. Other amino acids such as serine (S), glutamine (Q), asparagine (N) and histidine (H) might as well be used in their place.

Finally, as it has been extensively shown that “humanised” HIV-1 genes result in more efficient translation (29, 32, 39), codon usage was adapted according to that of Homo sapiens (http://www.kazusa.or.jp/codon). This is relevant as the codon usage of HIV-1 is highly biased in favour of A in the third base (15).

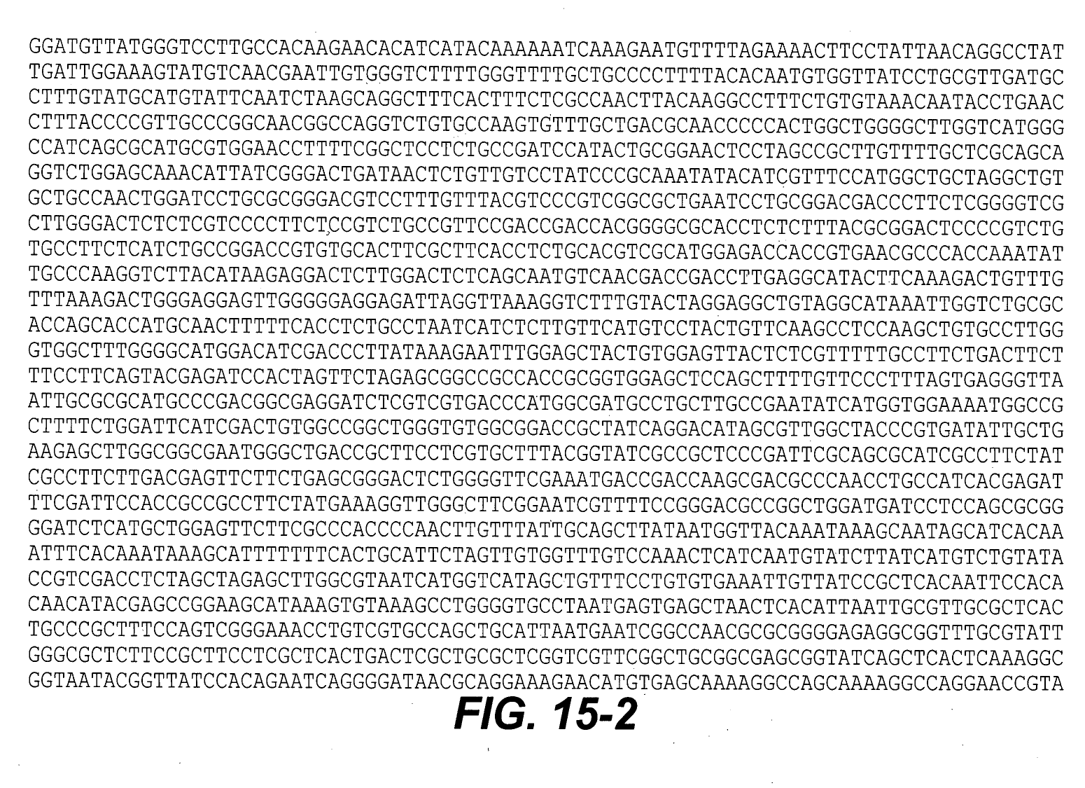

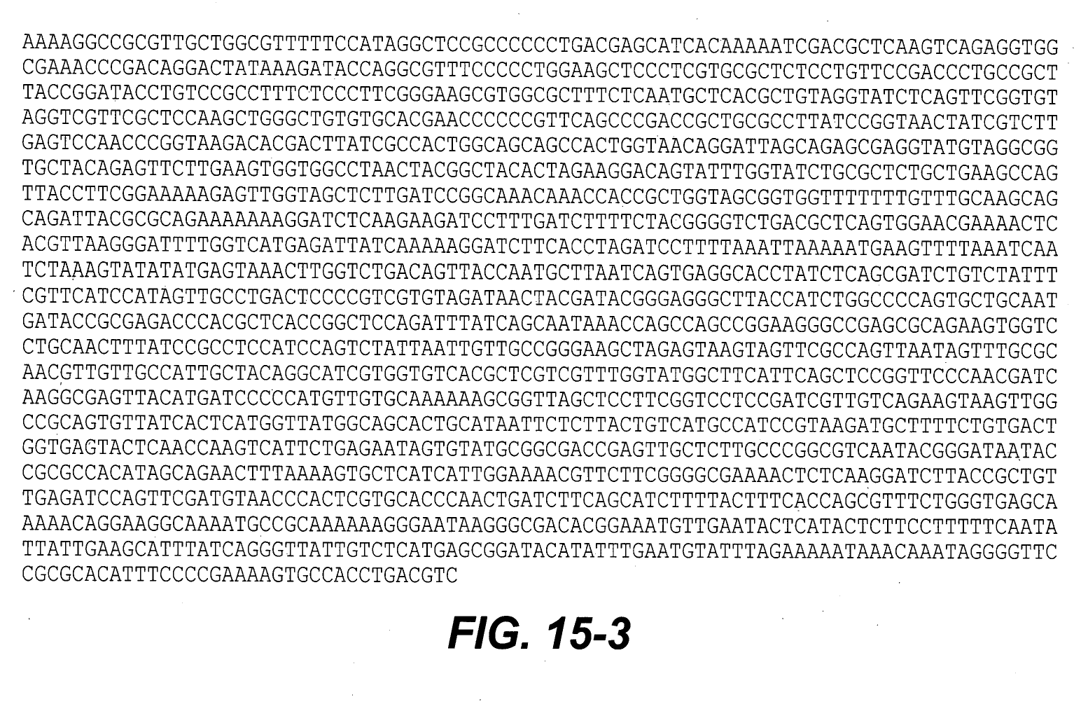

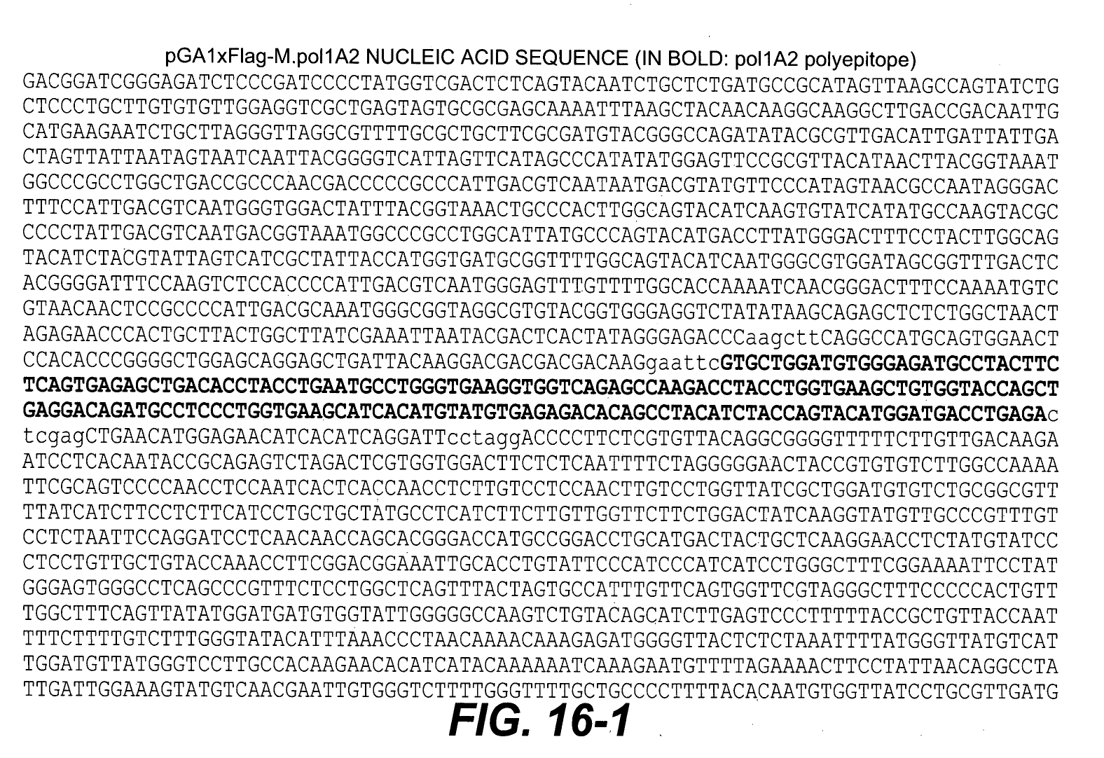

Comparison of the hydropathy profile of the original HIV-1 class I polyepitope sequence (FIG. 1D) to that of the redesigned polHIV-1.opt polyepitope (FIG. 1E) emphasises a clear enhancement of hydrophilicity. Indeed, the new profile is qualitatively closer to those for the preS1/preS2 peptides from the HBV strain used in the present invention (FIG. 1F) and 15 from numerous HBVs from primates and mammals (FIG. 5: ftp:/iftp.pasteur.fr/pub/retromol/Michel2006).

### Optimized Polyepitope VLPs are Secreted

The ppolHIV-1 and ppolHIV-1.opt plasmids were transiently transfected into SW480 cells, along with pCMV-basic and pCMV-S2.S as positive controls for HBsAg VLPs formation and secretion. The pCMV-S2.S plasmid expresses the wild type preS2-HBsAg fusion protein (23), while the pCMV-basic plasmid corresponds to the ppolHIV-1.opt construction, where the polHIV-1.opt polyepitope is substituted by a polylinker of five restriction sites. (FIG. 9.)

The ELISA test used allows detection and quantification of HBsAg antigenic units only if the protein is assembled into VLPs. The pCMV-basic and ppolHIV-1.opt plasmids resulted in VLPs secretion ˜5-50 fold down from the pCMV-S2.S (FIG. 2A). These data clearly show a gradual impact of fusion protein complexity on the inhibition of recombinant VLPs assembly. Nevertheless, over a 14 days period, recombinant HBsAg VLPs could be detected in supernatants from cultures transfected by ppolHIV-1.opt, in sharp contrast to the ppolHIV-1 construct, which failed to result in any detectable secretion whatsoever, on a par with the limits of detection (0.1 ng/ml) (FIG. 2A).

To verify that the ppolHIV-1.opt VLPs presented polyepitopes on their surfaces, an ELISA assay specific for the detection of the HIV-1 V3 loop tag was performed (FIG. 2B). The V3 loop is a linear epitope from the HIV-1 MN isolate inserted between the polyepitope and HBsAg (FIG. 1A). V3 loop ELISA was performed on the equivalent of 1.25 or 2.5 ng HBsAg/ml of supernatants. Results showed that the ppolHIV-1-opt construct did present the V3 loop epitope on the surface of HBsAg VLPs although values were ˜3-5 fold down compared to the pCMV-basic control (FIG. 2B). As to ppolHIV-1, even using as much as a maximum of undiluted supernatant for the ELISA assay (100 μl), no signal could be detected over the limit of detection (0.015 OD^{450nm}). These findings are internally consistent with the data from the anti-HBsAg ELISA assay (FIG. 2A).

Even though ppolHIV-1.opt was efficiently secreted, it was less than either of the control plasmids pCMV-S2.S and pCMV-basic. To explore whether there was an effect of the fusion protein on HBsAg secretion alone, ppolHIV-1.opt was cotransfected with pCMV-S2.S at two different stoichiometries. As can be seen from FIG. 2C, ppolHIV-1.opt exerted a trans-dominant inhibitory effect on HBsAg secretion in a dose dependent manner, indicating that the fusion protein was retaining some HBsAg, presumably in the cytoplasm.

### Optimized Polyepitope Vlps Result in a Diffuse Granular Intracytoplasmic Staining Like the Positive Control

VLPs detection by antibodies (Abs) in the ELISA assays (FIGS. 2A and 2B) might have been impaired by hydrophobic polHIV-1 polyepitope masking antigenic sites, notably in the V3 loop tag and the HBsAg. Alternatively, recombinant HBsAg proteins could be blocked in the secretory pathway. To explore this possibility, confocal immunofluorescence analysis was performed on the SW480 cell line transfected by ppolHIV-1, ppolHIV-1.opt, or pCMV-basic control plasmids. Using an anti-“a” HBV serotype determinant monoclonal antibodies (mAb) for the detection of HBsAg and polyclonal anti-giantin Abs for identifying the Golgi apparatus, this analysis showed a clear localisation of the HBsAg protein within the Golgi apparatus for ppolHIV-1 (FIG. 3A). In sharp contrast, for ppolHIV-1.opt, HBsAg appeared as largely diffused throughout the cytoplasm in punctate spots and HBsAg localisation within the Golgi apparatus was almost non-existent (FIG. 3B). Comparable punctate spots were nearly absent in ppolHIV-1 samples (FIG. 3A). As compared to the pCMV-basic control (FIG. 3C), where diffuse granular staining seems homogeneous in size, ppolHIV-1.opt red spots (FIG. 3B) showed remarkably different dimensions throughout cytoplasm, possibly reflecting sites of partial HBsAg retention sites (8).

ppolHIV-1.opt VLPs Induce Anti-HBsAg Neutralising Antibodies

In human, natural, HBV infection, most of anti-HBsAg neutralizing antibodies recognise conformationally dependent epitopes (22). In other words, they bind to HBsAg only if the antigen is assembled into VLPs. Hence, we sought in vivo the impact of VLPs secretion on anti-HBsAg humoral response was examined in vivo in HLA-A*0201 transgenic mice (HHD mice: HHD+/+ β2m−/− Db−/−; (11)) and both HLA-A*0201/HLADR1 double transgenic mice (HHD+/+ β2m−/− HLA-DR1+/+IAβ−/−; (26)). The choice of these two mice models is due to the fact that they ensure humanised class I and class II epitope presentation (11, 26).

Six HHD mice were immunized with either the ppolHIV-1 or the ppolHIV-1.opt constructions, and a boost was provided at day 11. Following sacrifice at day 23, sera were collected and tested by ELISA assay for the presence of anti-HBsAg conformational antibodies. Anti-HBsAg conformational immunoglobulin G (IgGs) titers in the sera (1:100 diluted) of three positive ppolHIV-1.opt immunized HHD mice were 2 to 2.5 fold higher than the mean value for non-immunized mice controls (FIG. 4A). Of six HHD mice immunized with the ppolHIV-1, all gave negative results. When repeated on groups of three HHD+/+β2m−/−HLA-DR1+/+IAβ−/− mice, comparable results were obtained. Two out of three ppolHIV-1.opt immunized mice presented anti-HBsAg conformational IgGs, showing values 2-fold higher than controls (FIG. 4B).

### Specific CD8⁺ T Cell Activation was Influenced by VLPs Secretion

The polHIV-1 and polHIV-1.opt polyepitopes determined different fates of the respective polyepitope-HBsAg fusion proteins. The polHIV-1 polyepitope impaired VLPs secretion (FIG. 2), leading to accumulation of the fusion protein in the Golgi apparatus (FIG. 3). Intra-cellular retention or secretion of fusion proteins was at the origin of opposite potentiality in eliciting anti-HBsAg humoral immune response. The anti-HBsAg neutralising humoral response has been shown to be CD4⁺ T cell-dependent (26).

To analyse the activation state of CD4⁺ T lymphocytes from ppolHIV-1 and ppolHIV-1.opt immunized mice, an IFN-γ secretion assay was performed on splenocytes from immunized and boosted HHD⁺/⁺ β2m⁻/⁻ HLA-DR1⁺/⁺IAβ⁻/⁻ mice (26). It has been demonstrated that this mouse model is a faithful animal model for epitope prediction and presentation in humans (27). Splenocytes were stimulated in vitro with the newly described Q16S and T15Q peptides corresponding to HLA-DR1-restricted HBsAg epitopes ((27) and unpublished data). Following stimulation with the two peptides, mean values were statistically similar (FIG. 4C). The T15Q stimulation induced a more uniformly positive response for ppolHIV-1.opt than for ppolHIV-1. Nevertheless, this test failed to show a statistically significant difference of CD4+ T cells activation between the two constructions. This might be explained by the fact that antigens released from destroyed transfected muscle cells are captured directly by APCs (9). Hence, myocytes of ppolHIV-1 immunized mice can release antigens at a sufficient level to induce CD4⁺ T cells activation at comparable level to that of ppolHIV-1.opt mice, in the absence of VLPs secretion.

In order to analyse the impact of recombinant HBsAg intra-cellular retention or secretion on eliciting anti-HIV-1 cellular immune response, an IFN-γ secretion assay by CD8⁺ T cells was performed. HHD mice were immunized and boosted with ppolHIV-1 or ppolHIV-1.opt and splenocytes were recovered at sacrifice. Cells were stimulated ex vivo by a combined total of 10 μg/ml of either one (S9L or V9V), two (S9L+L9V or L10V+V11V) or four (pool 1: L9V+L10V+S9L+Y/I9V or pool 2: V11V+Y/P9L+Y/V9L+Y/T9V) relevant peptides. Testing one or two peptides, with two mice per group, and performing the INF-γ release assay at day 0 and day 5, gave no specific secretion above background. This was not too surprising since the total preparation of CD8⁺ T lymphocytes in the HHD transgenic mice is about 1 to 4% of total splenic lymphocytes (in comparison CD8⁺ T cells represent ˜20% in C57BL6 mice, the genetic background where the HHD mice are derived from). In HHD mice, pools of four relevant peptides were needed to stimulate IFN-γ specific releases ex vivo (FIG. 4D). In this case, comparable results were obtained for the pool 1 epitopes, while significantly better IFN-γ secretion for ppolHIV-1.opt immunized mice resulted from the pool 2 stimulation. Globally, the optimized polHIV-1.opt polyepitope could induce higher levels of IFN-γ secreting activated HIV-1 specific CD8⁺ T lymphocytes.

### CTL Activity was Comparable for the Two Constructions

In order to compare the CTL immune response elicited by vaccination with the ppolHIV-1 or the ppolHIV-1.opt, nine mice per group were immunized and boosted with the two constructions. At sacrifice, spleens were collected from survivors for subsequent analyses. Splenocytes were re-stimulated in vitro at day 7 and the CTL specific activities evaluated by a ⁵¹Cr-release assay. The RMA-S HHD cell line stably transfected by the HLA-A*A0201 allele and sensitized with relevant or control peptides were used as target cells (Table 6).

TABLE 6
CTL specific activity directed against HLA-A*0201-restricted HIV-1 epitopes

| | epitope | | ppolHIV-1 | | ppolHIV-1.opt | |
|---|---|---|---|---|---|---|
| | origin | epitope^{a} | R/T^{b} | lysis^{c} | R/T | lysis |
| gag | p17 | S9L | 8/8 | 12; 15; 15 28; 36; 36; 43; 49 | 4/7 | 0; 5; 7; 11; 15; 17; 63 |
| | p24 | Y/T9V | 6/9 | 0; 1; 2; 12; 15; 17; 18; 31; 33 | 3/9 | 0; 0; 0; 1; 2; 2; 12; 27; 51 |
| pol | protease | L10V | 1/8 | 0; 0; 0; 1; 2; 7; 9; 11 | 1/7 | 0; 0; 0; 0; 1; 4; 14 |
| | RT | V11V | 4/8 | 0; 0; 2; 5; 16; 17; 25; 31 | 6/7 | 0; 14; 14; 17; 21; 25; 30 |
| | RT | Y/V9L | 0/9 | 0; 0; 0; 0; 0; 0; 0; 0; 0 | 2/9 | 0; 0; 0; 0; 0; 0; 1; 13; 27 |
| | RT | Y/I9V | 3/9 | 0; 0; 0; 2; 4; 6; 14; 18; 19 | 2/9 | 0; 0; 0; 0; 0; 1; 4; 22; 33 |
| | RT | Y/P9L | 9/9 | 16; 17; 18; 19; 19; 20; 21; 37; 38 | 2/9 | 0; 0; 0; 1; 8; 8; 9; 13; 51 |
| | integrase | L9V | 2/8 | 0; 0; 0; 3; 4; 4; 15; 15 | 0/7 | 0; 0; 0; 0; 0; 0; 0 |

^{a}see table 7 for peptide sequences
^{b}number of responders (R) versus tested (T) mice
^{c}percentage of specific lysis at 100:1 effector to target ratio; for positive values, the cut-off was ≧10

Responses to six out of the eight epitopes (Y/T9V, L10V, V11V, Y/V9L, Y/I9V and L9V) were detected at comparable levels for the ppolHIV-1 and ppolHIV-1.opt immunized mice.

As far as the S9L and Y/P9L epitopes are concerned, responses where less efficient for the ppolHIV-1.opt immunized mice. Nevertheless, some discrepancies were observed among present data and previous published data obtained by ppolHIV-1 vaccination (12). In particular, while immunogenicity of the S9L, Y/T9V, L10V and V11V epitopes was reproduced in the present study, opposite results were obtained for the Y/V9L, here inefficient. The L9V epitope gave slightly better results in the previous analysis (from intermediate to inefficient; (12)), while the Y/P9L from intermediate becomes strong in present data. Comparison is not possible for the Y/I9V, as it was not tested (12).

These discrepancies underline the difficulties to obtain relative reliable data in the HHD mice model by the ⁵¹Cr-release in vitro assay, probably due to the low proportion of CD8⁺ T cells among splenocytes in this transgenic animal model.

All these data taken together show that it is possible to make self-assembling, recombinant, HBsAg VLPs with up to 138 residues of heterologous protein, provided a certain number of features typical of preS1 and preS2 regions are preserved. Preservation of recombinant VLPs assembly was demonstrated to be essential to elicit antibodies directed against conformational HBsAg epitopes, which constitute the major component of humoral, anti-HBV immune responses. Moreover, efficient recombinant VLPs secretion induced higher activation state of HIV-1 specific CD8⁺ T lymphocytes.

This invention will now be further described in the following Examples.

### EXAMPLE 1

### Expression Vector and Constructions

Constructions are based on the expression vector pCMV-B10 (11, 16, 21). The polHIV-1.opt polyepitope was cloned between the EcoRI and XhoI restriction sites. Codon usage was optimized according to the Homo sapiens table (http://www.kazusa.or.jp/codon). Hydrophathy profiles were obtained by DNA Strider™ 1.2 (Kyte-Doolittle option).

The polyepitope was assembled by “atypical PCR.” Briefly, a series of six 70-80-mer oligonucleotides were synthesised corresponding to the plus strand and overlapped one another by ˜20 bases at both 5′ and 3′ ends (The oligonucleotides used in this invention are shown in Table 1: ftp://ftp.pasteur.fr/pub/retromol/Michel2006).

Two separate reactions (A and B) were performed using 50 pmols of HIVPOLY-1, -2 and -3, in reaction A, and HIVPOLY-4, -5 and -6 in B, respectively (Table 1). Then, 25 pmols of 5′conpmodifpc and 3′modifpoly were added in reactions A and B, respectively. Fifteen cycles of PCR were then performed.

PCR products from reactions A and B were assembled as follows: 0.5 μl of each reaction were put in 20 μl of H₂O at 95° C. for 30 seconds and then to room temperature (RT). Five units of Klenow fragment and 1 μl of dNTPs (40 mM) were added and reaction performed for 15 minutes at 37° C.

Then, 25 cycles of classical PCR were performed, adding 100 pmols of the 5′modifpoly and the 3′modifpoly primers.

As a negative control, a derivative of the pCMV-B10 construction was made with a small polylinker (NheI, EcoRV, SmaI; see FIG. 9) replacing the pCMV-B10 polylinker between the EcoRI and XhoI restriction sites. This plasmid was referred to as pCMV-basic.

### EXAMPLE 2

### In Vitro Evaluation of VLPs Secretion

The SW480 human cell line was maintained in Dulbecco medium supplemented with 5% foetal calf serum (FCS) and 1% streptomycin and penicillin, according to recommendations of the manufacturer. The pCMV-S2.S plasmid was kindly provided by Dr. Marie-Louise Michel (23).

Cells were transiently transfected by FuGENE6™ transfection reagent (Roche). Out of 2 ml, 500 μl of supernatant were collected and renewed at each time point. HBsAg concentration in supernatants was estimated by the Monolisa® Ag HBsAg Plus Kit (BIORAD). The anti-HIV-1 V3 loop ELISA was performed using the F5.5 monoclonal antibody (F5.5 mAb; HybridoLab), which recognises a linear epitope. Briefly, 96 well plates were coated with F5.5 mAb, and 1.25 and 2.5 ng/ml of HBsAg positive samples tested per well. Positive wells were revealed by peroxidase reaction and read at 450 nm.

### EXAMPLE 3

### Immunofluorescence Analysis

The SW486 cell line was transfected by plasmids using the FuGENE6™ reagent (Roche). Four days later, cells were transferred to collagen treated coverslips and fixed the following day with 4% paraformaldehyde in PBS for 20 minutes at RT and then permabilized with 0.05% saponin, 0.2% bovine serum albumin (BSA) in PBS for 15 minutes. Cells were sequentially incubated for 1 hour at RT with primary and secondary Ab, diluted 1/100 and 1/2000, respectively, in 0.05% saponin, 0.2% BSA in PBS. Rabbit primary polyclonal Ab anti-giantin (BAbCO: PRB-114C), anti rabbit-alexa 488 secondary Ab (Molecular Probes: A11034), mouse primary mAb anti-HBsAg (DAKO: #3E7, M3506), and anti-mouse-alexa 568 secondary Ab (Molecular Probes: A11019) were used for Golgi and HBsAg labelling. Cellular nucleic acids were counterstained with 0.1 μg/ml of 4′,6-diamidino-2-phenylindole (DAPI: Sigma). Immunostained coverslips were then mounted on slides in Vectashield (Vector: H1000). Images were acquired on the LSM 510 Zeiss AXIOVERT 200 M confocal microscope piloted by a version 3.2 software, using the plan-APOCHROMAT×63 1.4 N.A. Alexa 488 was excited by an argon laser at 488 nm and the fluorescence emission collected through the BP 505-550 filter, alexa 568 by a HeNe laser at 543 nm and a LP 560 filter, and DAPI by a blue Diode laser at 405 nm and a BP 435-485 filter. Images were then exported as TIF files and subsequently treated by Adobe Photoshop CS 8.0.1.

### EXAMPLE 4

### Immunization of Mice

Immunization was performed on 8 to 10 weeks old HLA-A*0201 transgenic mice (HHD mice: HHD⁺/⁺ β2m⁻/⁻ Db−/−; (11)) or both HLA-A*0201 and HLA-DR1 double transgenic mice (HHD⁺/⁺ β2m⁻/⁻ HLA-DR1⁺/⁺ IAβ⁻/*; (26)) mice. Male and female mice were uniformly represented in groups. Plasmid DNA for immunization was prepared by endotoxin-free giga-preparation kit (QIAGEN) and re-suspended in endotoxin-free PBS (Sigma). Five days before DNA injection, an inflammatory reaction was induced by inoculating 1 nmol of cardiotoxin (Latoxan) per hind leg. At day 0, intramuscular immunization was performed by injecting 50 μg of plasmid DNA per hind leg, and at day 11, a 50 μg DNA boost per leg was made. At day 23, mice were sacrificed. Blood was collected by intra-heart puncture, heparinized and centrifuged 5 minutes at 3000 rpm. The overlaying serum was stored at 4° C. Splenocytes were re-suspended in RPMI medium supplemented with 5% foetal calf serum (FCS) and 1% streptomycin and penicillin.

### EXAMPLE 5

### ELISA Detection of Anti-HBsAg Antibodies

Nunc Maxisorp plates (Nunc) were coated with 100 μl of pure HBsAg VLPs (HyTest) at 1 μg/ml for 1 night at RT. HBsAg was of the same subtype (ayw) as that expressed by ppolHIV-1 and ppolHIV-1.opt. After washing with PBS-0.1% Tween-20, 200 μl of carbonate buffer pH 9.6 supplemented with 10% FCS was added per well and left overnight at RT. Serial dilutions of mice serum or the anti-HBsAg mAb (clone NE3, HyTest) were added to wells and incubated overnight at RT. Secondary Ab was the polyclonal anti-mouse IgG (Amersham: NXA931) labelled with peroxidase (Amersham). Following peroxidase reaction, wells were read at 490 nm. Non-immunized mice serum in duplicate gave the cut-off value for each plate. The anti-HBsAg mAb (clone NE3, HyTest) allowed determination of positive control values.

### EXAMPLE 6

### INF-γ Secretion Assay

INF-γ secretion assay was performed following the instructions of the manufacturer (Miltenyi Biotec). Briefly, following sacrifice, mice spleens were collected and re-suspended in RPMI medium. Splenocyte suspensions were transferred onto FicollYL and centrifuged 20 minutes at 2500 rpm. FicollYL was prepared mixing solution 1 (521.14 ml Telebrix 35, Guerbet laboratory, plus 547 ml H₂O) and solution 2 (225 g Ficoll PM400, Pharmacia Amersham Bioscience, plus 2.5 l H₂O), obtaining the final density of 1.076. FicollYL was sterilised and conserved at 4° C. Splenocytes were recovered at interphase, washed in RPMI, counted and resuspended at 10×10⁶ cells in 1 ml of RPMI supplemented with 3% FCS. Cells were then incubated at 37° C. for 16 hours with relevant or irrelevant peptides (10 μg/ml; Table 7: ftp://ftp.pasteur.fr/pub/retromol/Michel2006).

TABLE 7
HIV-1, HBsAg and influenza A peptides
restricted by HLA-A*0201 or HLA-DR1 alleles

| | | | | | |
|---|---|---|---|---|---|
| | | | | % frequencies in | |
| | | | | HIV-1 clade A, B | |
| Name | Sequence | Origin | Position | and C | Reference |

HLA-A′0201

| | | | | | |
|---|---|---|---|---|---|
| SyL | SLYNTVATL | gag (p17) | 77-85 | 56, 46, 33 | [First, 2001 U7] |
| L9V | LLWKGEGAV | pol (integrase) | 19-27 | 89, 100, 93 | [First, 2001 U7] |
| V11V | LLDTGADDTV | pol (protease) | 79-88 | 100, 100, 87 | [First, 2001 U7] |
| Y/V9L | YITOYMDDL | pol (RT) | 203-273 | 89, 90, 93 | [First, 2001 U7] |
| Y/P9L | YLVKLWYQL | pol (RT) | 464-472 | 89, 100, 93 | [First, 2001 U7] |
| Y/I9V | YLKEPVHGV | pol (RT) | 576-584 | 11, 76, 80 | [First, 2001 U7] |
| Y/T9V | YLNAWVKVV | gag (p24) | 956-964 | 11, 83, 20 | [First, 2001 U7] |
| G9L | GILGFVFTL | influenza A M1 | 56-66 | na | [First, 2001 U7] |

HLA-DR1

| | | | | | |
|---|---|---|---|---|---|
| Q165 | QAGFFLLTRILTIPQS | HBsAg | 179-194 | na | [Pajot 2004 N5] |
| T15Q | TSLNFLGGTTVCLGQ | HBsAg | 200-214 | na | ″ |
| P13T | PKYVKQNTLKLAT | influenza A HA1 | 306-318 | [Pajot 2004 No] | |

* Not-applicable
** Personal communication from Lone Yu Chun

The irrelevant G9L and P13T peptides were used as negative controls in INF-γ secretion assay by CD8⁺ T cells and CD4⁺ T cells, respectively. For positive control samples, 12.5 ng/ml phorbol 12-myristate 13-acetate (PMA; Sigma) and 1 μg/ml ionomycin (Sigma) were added to cells. Samples were labelled with INF-γ catch reagent and then with the INF-γ-PE detection Ab, and the CD8a-APC (clone 53-6.7; Miltenyi Biotec) or with the CD4-FITC (clone GK1.5; Miltenyi Biotec) antibodies. Samples were analysed by the flow cytometry analysis using a FACScalibur (BD-Biosciences). Secretion percentages corresponding to the irrelevant peptides were subtracted from values obtained with the relevant peptides. p values were obtained by the StatView F-4.5 using the Mann-Whitney non-parametric test.

### EXAMPLE 7

### CTL Assays on Immunized Mice Splenocytes

Following FicollYL, LPS-blasts from two naïve spleens were cultivated at 37° C. for 3 days in 50 ml RPMI supplemented with 10% FCS, 2% streptomycin and penicillin, 1% glutamine (GIBCO BRL), 0.05 mM β-mercaptoethanol, 25 μg/ml LPS (5 mg/ml; Sigma), 7 μg/ml dextran sulphate (7 mg/ml; Sigma). Splenocytes from immunized HHD mice were cultured at 5×10⁶/ml and stimulated for 7 days by irradiated LPS-blast cells loaded with HLA-A*0201-restricted peptides at effector-presenting cell ratio of 1:1. CTL specific activity of effector cells was tested against HLA-A*0201 stably transfected target cells (RMA-S HHD cell line), (28) pulsed with 10 μg/ml of each of the HLA-A*0201-restricted peptides (Table 2) and previously incubated with ⁵¹Cr (5 mCi/ml Amersham) for 1 hour at 37° C. Effector and target cells were mixed at 100:1, 60:1, and 30:1 ratios and then incubated for 4 hours at 37° C. Fifty microlitres of supernatants were harvested from centrifuged plates, loaded on a Lumaplate (PerkinElmer) and counted with a beta counter following overnight incubation at 37° C. (7). Spontaneous and maximum ⁵¹Cr-release were determined with RMA-S HHD samples supplemented with culture medium or 1% bleach. CTL specific activity was estimated as the mean value of triplicates following the formula: (experimental-spontaneous release)/(maximum-spontaneous release)×100. Results were considered positive if specific lysis was more than 10% and the 100:1 ratio was chosen as the best representative data.

In summary, many bivalent vaccine candidates have been based on the fusion of immunogenic peptides to the HBsAg carrier (2, 12, 19, 21, 27, 30). Data presented here shows that the design of fusion protein must preserve HBsAg-driven VLPs assembly. Numerous parameters were incorporated into the design of the polHIV-1.opt polyepitope and so it is difficult to pinpoint any one as being dominant. As the preS1/preS2 peptides of mammalian HBVs are generally hydrophilic, lack of cysteine and methionine residues, all three parameters are probably important. Once these features were taken into account, VLPs secretion was increased at least 120 fold (FIG. 2A). Adapting the HIV-1 polyepitope codon usage to that of Homo sapiens probably contributed to overall HBsAg translation in line with numerous reports (29, 32, 39). However, this would not impact VLPs assembly.

The confocal immunofluorescence analysis clearly showed that the highly hydrophobic polHIV-1 polyepitope resulted in massive accumulation of HBsAg in the Golgi apparatus (FIG. 3A). In turn, this analysis ruled out masking of the HBsAg serotype “a” determinant or of the V3 loop epitope in the fusion protein by the hydrophobic polypepitope in the ELISA assays (FIG. 2). Notably, in the 22 confocal immunofluorescence analysis, the Golgi apparatus was labelled by polyclonal Abs directed against the giantin protein. Giantin is a membrane-inserted component of the cis and medial Golgi, with a large rod-like cytoplasmic domain. Hence, only the Golgi compartments positioned nearer to the underlying ER are stained, while the trans Golgi network is not visible. In the ppolHIV-1 transfected SW480 cells (FIG. 3A), focal planes corresponding to the cis Golgi network showed the largest HBsAg red granular spots, while the smallest were more distal from the ER (data not shown) and of the same size of the few visualised out of the Golgi apparatus. This seems to indicate major and early retention following HBsAg trafficking trough the ER. HBsAg retention in the Golgi apparatus of ppolHIV-1 transfected cells was comparable to that obtained by the L77R HBsAg mutant (8). In this invention, retention of the ppolHIV-1 HBsAg in the Golgi paralleled reduced levels of extracellular HBsAg detection by ELISA assay.

The present invention shows that residues in the N-terminal region of the recombinant HBsAg protein too strongly impact Golgi retention and VLPs secretion. By confocal microscopic analysis, the polyepitope optimization resulted in HBsAg diffuse cytoplasmic granular staining similar to that obtained with the pCMV-basic control plasmid. The higher frequency of relatively larger red intracytoplasmic punctate spots (FIG. 3B) suggests that some fraction of HBsAg from ppolHIV-1.opt could be further intracellularly retained compared to the control. That ppolHIV-1.opt proved to exert a trans-dominant negative effect on HBsAg secretion (FIG. 2C) is in agreement with the notion that the red punctate spots represent intra-cytoplasmic sites of HBsAg retention.

Ex vivo evaluation of the activation state of HIV-1 specific CD8⁺ T lymphocytes globally showed higher activation in the ppolHIV-1.opt samples than in the ppolHIV-1 (FIG. 4D). Aside from these data, the in vitro analysis of the effector activity of these CD8⁺ T cells gave comparable results for the two constructions (Table 6). This later analysis was based on a ⁵¹Cr-release assay which requires the loading of substantial quantities of peptide on presenting cells (RMA-S HHD) to elicit their lysis by epitope specific CTLs. This could be at the origin of the apparent discrepancy between the two tests used to characterise the Th1 response against HIV-1 epitopes. Over-presentation of epitopes can engage apoptosis events in HIV-1 specific activated CD8⁺ T cells rather than effector activity. Indeed, it has been demonstrated that over-expression of an epitope can adversely affect the quality of T cell response (6). Moreover, the ⁵¹Cr-release test follows one week of in vitro culture of splenocytes, where the subtle equilibrium between quantity and quality of antigen specific CD8⁺ T cells might be altered.

Nevertheless, by using pools of epitopes (which mimics the situation in vivo, being epitopes delivered to mice as polyepitope) and ex vivo analysis of cells, the INF-γ secretion assay gave a reliable picture of the significant better activation state of HIV-1 specific CD8⁺ T cells in ppolHIV-1.opt samples. In combination with MHC binding affinity, epitope density has been demonstrated to influence the amplitude and the quality of CD8⁺ T cells response in vivo (6, 41). Optimization parameters allowing assembly of recombinant VLPs can ensure a better antigen density for uptake and APCs cross-presentation (1, 17, 33-35, 38). This could induce the enhancement of the activation state of the HIV-1 specific CD8⁺ T cell populations in mice immunized with the optimized construction.

The ELISA detection assay for in vivo anti-HBsAg antibody production positively selected for IgGs directed against conformational epitopes (FIGS. 4A and 4B). Most neutralising anti-HBsAg antibodies, an essential component in the immune response against natural infection by human HBVs, recognise conformational epitopes on HBsAg VLPs (23). Hence, B lymphocytes of immunized mice could encounter conformational epitopes only if immunized by the ppolHIV-1.opt. In the ppolHIV-1 immunized mice, HBsAg epitopes eliciting humoral responses might have resulted from the releasing of antigen-producing cell debris (e.g. myocytes) consequent to their destruction (9, 13, 31). Yet, in that case, HBsAg folding in association to ER and Golgi membranes did not allow constitution of conformational epitopes and therefore production of neutralising antibodies. The results obtained according to this invention correlate with previous data showing that the development of humoral responses depends on the location of the antigen and the route of immunization (4, 18, 25). Particularly, in the context of intramuscular immunization, the same antigen (ovalbumin) elicited different immune responses whether it was cytoplasmic, transmembrane or secreted (25). As expected, only the secreted ovalbumin form could induce antibodies production.

In conclusion, the present invention shows that it is possible to make self-assembling recombinant HBsAg VLPs with residues of heterologous protein, provided a certain number of features typical of naturally occurring preS1 and preS2 regions are respected. Preservation of recombinant VLPs assembly was demonstrated to be essential to elicit antibodies directed against conformational HBsAg epitopes, which constitute the major component of humoral anti-HBV immune responses. Moreover, efficient recombinant VLPs secretion induced higher activation state of HIV-1 specific CD8⁺ T lymphocytes.

The following plasmids were deposited at the Collection Nationale de Cultures de Microorganismes (C.N.C.M.), of Institut Pasteur, 25 rue du Docteur Roux, F-75724 Paris, Cedex 15, France, and assigned the following Accession Nos.:

| Plasmid | Accession No. |
|---|---|
| pGA1xFlag-M | CNCM I-3543 filed on Dec. 16, 2005 |
| pGA1xFlag-Mpol.opt | CNCM I-3544 filed on Dec. 16, 2005 |
| pGA3xFlag-M | CNCM I-3545 filed on Dec. 16, 2005 |
| pGA3xFlag-Mpol.opt | CNCM I-3546 filed on Dec. 16, 2005 |
| ppolHIV-1.opt | CNCM I-3547 filed on Dec. 16, 2005 |
| pGA1xFlag-M.pol 1A2 | CNCM I-3579 filed on Feb. 28, 2006 |
| pGA1xFlag-M.pol 2A2 | CNCM I-3580 filed on Feb. 28, 2006 |
| pGA1xFlag-M.pol.1B7 | CNCM I-3581 filed on Feb. 28, 2006 |
| pGA1xFlag-M.pol 2B7 | CNCM I-3582 filed on Feb. 28, 2006. |

### REFERENCES

The following references are cited herein. The entire disclosure of each reference is relied upon and incorporated by reference herein.
1. Albert, M. L., S. F. Pearce, L. M. Francisco, B. Sauter, P. Roy, R. L. Silverstein, and N. Bhardwaj. 1998. Immature dendritic cells phagocytose apoptotic cells via alphavbeta5 and CD36, and cross-present antigens to cytotoxic T lymphocytes. J. Exp. Med. 188:1359-1368.
2. Bisht, H., D. A. Chugh, M. Raje, S. S. Swaminathan, and N. Khanna. 2002. Recombinant dengue virus type 2 envelope/hepatitis B surface antigen hybrid protein expressed in Pichia pastoris can function as a bivalent immunogen. J. Biotechnol. 99:97-110.
3. Boisgerault, F., G. Moron, and C. Leclerc. 2002. Virus-like particles: a new family of delivery systems. Expert Rev. Vaccines 1:101-109.
4. Boyle, J. S., C. Koniaras, and A. M. Lew. 1997. Influence of cellular location of expressed antigen on the efficacy of DNA vaccination: cytotoxic T lymphocyte and antibody responses are suboptimal when antigen is cytoplasmic after intramuscular DNA immunization. Int. Immunol. 9:1897-1906.
5. Bruss, V. 2004. Envelopment of the hepatitis B virus nucleocapsid. Virus Res. 106:199-209.
6. Bullock, T. N., T. A. Colella, and V. H. Engelhard. 2000. The density of peptides displayed by dendritic cells affects immune responses to human tyrosinase and gp100 in HLA-A2 transgenic mice. J. Immunol. 164:2354-2361.
7. Buseyne, F., M. Fevrier, S. Garcia, M. L. Gougeon, and Y. Riviere. 1996. Dual function of a human immunodeficiency virus (HIV)-specific cytotoxic T-lymphocyte clone: inhibition of HIV replication by noncytolytic mechanisms and lysis of HIV-infected CD4+ cells. Virology 225:248-253. 30
8. Chua, P. K., R. Y. Wang, M. H. Lin, T. Masuda, F. M. Suk, and C. Shih. 2005. Reduced secretion of virions and hepatitis B virus (HBV) surface antigen of a naturally occurring HBV variant correlates with the accumulation of the small s envelope protein in the endoplasmic reticulum and Golgi apparatus. J. Virol. 79:13483-13496.
9. Davis, H. L., C. L. Millan, and S. C. Watkins. 1997. Immune-mediated destruction of transfected muscle fibers after direct gene transfer with antigen-expressing plasmid DNA. Gene Ther. 4:181-188.
10. Doan, L. X., M. Li, C. Chen, and Q. Yao. 2005. Virus-like particles as HIV-1 vaccines. Rev. Med. Virol. 15:75-88.
11. Firat, H., F. Garcia-Pons, S. Tourdot, S. Pascolo, A. Scardino, Z. Garcia, M. L. Michel, R. W. Jack, G. Jung, K. Kosmatopoulos, L. Mateo, A. Suhrbier, F. A. Lemonnier, and P. Langlade-Demoyen. 1999. H-2 class I knockout, HLA-A2.1-transgenic mice: a versatile animal model for preclinical evaluation of antitumor immunotherapeutic strategies. Eur. J. Immunol. 29:3112-3121.
12. Firat, H., S. Tourdot, A. Ureta-Vidal, A. Scardino, A. Suhrbier, F. Buseyne, Y. Riviere, O. Danos, M. L. Michel, K. Kosmatopoulos, and F. A. Lemonnier. 2001. Design of a polyepitope construct for the induction of HLA-A0201-restricted HIV 1-specific CTL responses using HLA-A*0201 transgenic, H-2 class I KO mice. Eur. J. Immunol. 31:3064-3074.
13. Inaba, K., S. Turley, F. Yamaide, T. Iyoda, K. Mahnke, M. Inaba, M. Pack, M. Subklewe, B. Sauter, D. Sheff, M. Albert, N. Bhardwaj, I. Mellman, and R. M. Steinman. 1998. Efficient presentation of phagocytosed cellular fragments on the major histocompatibility complex class II products of dendritic cells. J. Exp. Med. 188:2163-2173.
14. Kozak, M. 2002. Pushing the limits of the scanning mechanism for initiation of translation. Gene 299:1-34.
15. Kypr, J., and J. Mrazek. 1987. Unusual codon usage of HIV. Nature 327:20.
16. Le Borgne, S., M. Mancini, R. Le Grand, M. Schleef, D. Dormont, P. Tiollais, Y. Riviere, and M. L. Michel. 1998. In vivo induction of specific cytotoxic T lymphocytes in mice and rhesus macaques immunized with DNA vector encoding an HIV epitope fused with hepatitis B surface antigen. Virology 240:304-315.
17. Lenz, P., C. D. Thompson, P. M. Day, S. M. Bacot, D. R. Lowy, and J. T. Schiller. 2003. Interaction of papillomavirus virus-like particles with human myeloid antigen presenting cells. Clin. Immunol. 106:231-237.
18. Lewis, P. J., H. van Drunen Littel-van den, and L. A. Babiuk. 1999. Altering the cellular location of an antigen expressed by a DNA-based vaccine modulates the immune response. J. Virol. 73:10214-10223.
19. Li, H. Z., H. Y. Gang, Q. M. Sun, X. Liu, Y. B. Ma, M. S. Sun, and C. B. Dai. 2004. Production in Pichia pastoris and characterization of genetic engineered chimeric HBV/HEV virus-like particles. Chin. Med. Sci. J. 19:78-83.
20. Livingston, B. D., M. Newman, C. Crimi, D. McKinney, R. Chesnut, and A. Sette. 2001. Optimization of epitope processing enhances immunogenicity of multiepitope DNA vaccines. Vaccine 19:4652-4660.
21. Marsac, D., A.-L. Puaux, Y. Riviere, and M. L. Michel. 2005. In vivo induction of cellular and humoral immune response by hybrid DNA vectors encoding simian/human immunodeficiency virus/hepatitis B surface antigen virus particles in BALB/c and HLA-A2-transgenic mice. Immunobiology 210:305-319.
22. Mathet, V. L., M. Feld, L. Espinola, D. O, Sanchez, V. Ruiz, O. Mando, G. Carballal, J. F. Quarleri, F. D'Mello, C. R. Howard, and J. R. Oubina. 2003. Hepatitis B virus S gene mutants in a patient with chronic active hepatitis with circulating Anti-HBs antibodies. J Med Virol 69:18-26.
23. Michel, M. L., H. L. Davis, M. Schleef, M. Mancini, P. Tiollais, and R. G. Whalen. 1995. DNA-mediated immunization to the hepatitis B surface antigen in mice: aspects of the humoral response mimic hepatitis B viral infection in humans. Proc. Natl. Acad. Sci. USA 92:5307-5311.
24. Michel, M. L., and D. Loirat. 2001. DNA vaccines for prophylactic or therapeutic immunization against hepatitis B. Intervirology 44:78-87.
25. Morel, P. A., D. Falkner, J. Plowey, A. T. Larregina, and L. D. Falo. 2004. DNA immunization: altering the cellular localisation of expressed protein and the immunization route allows manipulation of the immune response. Vaccine 22:447-456.
26. Pajot, A., M. L. Michel, N. Fazilleau, V. Pancre, C. Auriault, D. M. Ojcius, F. A. Lemonnier, and Y. C. Lone. 2004. A mouse model of human adaptive immune functions: HLA-A2.1-/HLA-DR1-transgenic H-2 class I-/class II-knockout mice. Eur. J. Immunol. 34:3060-3069.
27. Pajot, A., V. Pancre, N. Fazilleau, M. L. Michel, G. Angyalosi, D. M. Ojcius, C. Auriault, F. A. Lemonnier, and Y. C. Lone. 2004. Comparison of HLA-DR1-restricted T cell response induced in HLA-DR1 transgenic mice deficient for murine MHC class II and HLA-DR1 transgenic mice expressing endogenous murine MHC class II molecules. Int. Immunol. 16:1275-1282.
28. Pascolo, S., N. Bervas, J. M. Ure, A. G. Smith, F. A. Lemonnier, and B. Perarnau. 1997. HLA-A2.1-restricted education and cytolytic activity of CD8(+) T lymphocytes from beta2 microglobulin (beta2m) HLA-A2.1 monochain transgenic H-2Db beta2m double knockout mice. J. Exp. Med. 185:2043-2051.
29. Peixoto, L., A. Zavala, H. Romero, and H. Musto. 2003. The strength of translational selection for codon usage varies in the three replicons of Sinorhizobium meliloti. Gene 320:109-116.
30. Pumpens, P., R. Razanskas, P. Pushko, R. Renhof, I. Gusars, D. Skrastina, V. Ose, G. Borisova, I. Sominskaya, I. Petrovskis, J. Jansons, and K. Sasnauskas. 2002. Evaluation of HBs, HBc, and frCP virus-like particles for expression of human papillomavirus 16 E7 oncoprotein epitopes. Intervirology 45:24-32.
31. Rajcani, J., T. Mosko, and 1. Rezuchova. 2005. Current developments in viral DNA vaccines: shall they solve the unsolved? Rev. Med. Virol. 15:303-325.
32. Romero, H., A. Zavala, H. Musto, and G. Bernardi. 2003. The influence of translational selection on codon usage in fishes from the family Cyprinidae. Gene 317:141-147.
33. Rudolf, M. P., S. C. Fausch, D. M. Da Silva, and W. M. Kast. 2001. Human dendritic cells are activated by chimeric human papillomavirus type-16 virus-like particles and induce epitope-specific human T cell responses in vitro. J. Immunol. 166:5917-5924.
34. Rudolf, M. P., J. D. Nieland, D. M. DaSilva, M. P. Velders, M. Muller, H. L. Greenstone, J. T. Schiller, and W. M. Kast. 1999. Induction of HPV16 capsid protein-specific human T cell responses by virus-like particles. Biol. Chem. 380:335-340.
35. Ruedl, C., T. Storni, F. Lechner, T. Bachi, and M. F. Bachmann. 2002. Cross-presentation of virus-like particles by skin-derived CD8(−) dendritic cells: a dispensable role for TAP. Eur. J. Immunol. 32:818-825.
36. Schreckenberger, C., and A. M. Kaufmann. 2004. Vaccination strategies for the treatment and prevention of cervical cancer. Curr. Opin. Oncol. 16:485-491.
37. Stern, P. L. 2005. Immune control of human papillomavirus (HPV) associated anogenital disease and potential for vaccination. J. Clin. Virol. 32 Suppl 1:S72-81.
38. Subklewe, M., C. Paludan, M. L. Tsang, K. Mahnke, R. M. Steinman, and C. Munz. 2001. Dendritic cells cross-present latency gene products from Epstein-Barr virus-transformed B cells and expand tumor-reactive CD8(+) killer T cells. J. Exp. Med. 193:405-411.
39. Wang, S., D. J. Farfan-Arribas, S. Shen, T. H. Chou, A. Hirsch, F. He, and S. Lu. 2005. Relative contributions of codon usage, promoter efficiency and leader sequence to the antigen expression and immunogenicity of HIV-1 Env DNA vaccine. Vaccine. In Press.
40. Wang, Y., J. A. Smith, T. Kamradt, M. L. Gefter, and D. L. Perkins. 1992. Silencing of immunodominant epitopes by contiguous sequences in complex synthetic peptides. Cell. Immunol. 143:284-297.
41. Wherry, E. J., M. J. McElhaugh, and L. C. Eisenlohr. 2002. Generation of CD8(+) T cell memory in response to low, high, and excessive levels of epitope. J. Immunol. 168:4455-4461.
42. Yan, M., J. Peng, I. A. Jabbar, X. Liu, L. Filgueira, I. H. Frazer, and R. Thomas. 2004. Despite differences between dendritic cells and Langerhans cells in the mechanism of papillomavirus-like particle antigen uptake, both cells cross-prime T cells. Virology 324:297-310.
43. Buck C B, Pastrana D V, Lowy D R, Schiller J T. Generation of HPV pseudovirions using transfection and their use in neutralization assays. Methods Mol Med 2005; 119:445-62)
44. Mason H S, Ball J M, Shi J J, Jiang X, Estes M K, Arntzen C J. Expression of Norwalk virus capsid protein in transgenic tobacco and potato and its oral immunogenicity in mice. Proc Natl Acad Sci USA. 1996 May 28; 93(11):5335-40.
45. Roth J F. The yeast Ty virus-like particles. Yeast 2000; 16(9):785-95),
46. Sedlik C, Saron M, Sarraseca J, Casal I, Leclerc C. Recombinant parvovirus-like particles as an antigen carrier: a novel nonreplicative exogenous antigen to elicit protective antiviral cytotoxic T cells. Proc Natl Acad Sci USA 1997; 94(14):7503-8),
47. Yang H J, Chen M, Cheng T, He S Z, Li S W, Guan B Q, et al. Expression and immunoactivity of chimeric particulate antigens of receptor binding site-core antigen of hepatitis B virus. World J Gastroenterol 2005; 11 (4):492-97),

What is claimed is:

1. An expression vector for the production of virus-like particles comprising fusion proteins and S proteins of hepatitis B virus (HBV), wherein the proteins are encoded by the preS2+S regions and S region of the HBV genome, respectively, and wherein the expression vector comprises
a polynucleotide that encodes a polypeptide comprising a heterologous polyepitopic sequence of interest, wherein epitopes in the polyepitopic sequence are in head to tail position, wherein the polynucleotide sequence is positioned in the preS2 region downstream of the preS2 ATG codon, and wherein the polynucleotide sequence is free of codons for cysteine and contains as few codon for methionine as possible;
polynucleotides encoding tetra-amino acid spacers between the head to tail epitopes in the polyepitopic sequence, wherein each spacer comprises an arginine (R) residue placed in the epitope C₁-position directly linked to a sequence of three different amino acids independently selected from alanine (A), threonine (T), lysine (K), and aspartic acid (D);
wherein preS2 translation initiation codon and S translation initiation codon are preserved so that S protein and the fusion protein comprised of M protein and the polypeptide comprising the polyepitopic sequence are translated, such that the S proteins and the fusion proteins assemble into virus-like particles after expression of the vector in a host cell.

2. The vector as claimed in claim 1, wherein the polyepitopic sequence of interest is from a pathogen

3. The vector as claimed in claim 2, wherein the pathogen is human immunodeficiency virus.

4. The vector as claimed in claim 1, wherein the polynucleotide sequence is free of methionine codons.

5. The vector as claimed in claim 1, wherein the polynucleotide sequence encodes polHIV-1.opt.

6. A host cell comprising the vector as claimed in claim 1.

7. A method of producing virus-like particles, wherein the method comprises:
providing a host cell as claimed in claim 6; and
expressing the fusion protein and the S protein under conditions in which the proteins assemble into virus-like particles, which are released from the host cell into extracellular space.

8. Virus-like particles comprising
fusion proteins and S proteins of hepatitis B virus, wherein the proteins are encoded by modified-preS2+S regions and S region, respectively, of the HBV genome;
a polypeptide fused in-frame in the M protein downstream of the preS2 translation initiation methionine residue, wherein the polypeptide is free of cysteine residues and contains 0 or 1 methionine residues, and wherein the polypeptide comprises a polyepitopic sequence of interest, wherein epitopes in the polyepitopic sequence are in head to tail position;
tetra-amino acid spacers between the head to tail epitopes in the polypeptide sequence, wherein each spacer comprises an arginine (R) residue placed in the epitope C₁-position followed by three different amino acids independently selected from alanine (A), threonine (T), lysine (K), and aspartic acid (D);
wherein the S proteins and the fusion proteins are assembled into the virus-like particles.

9. The virus-like particles as claimed in claim 8, wherein the polypepitopic sequence of interest comes from a human immunodeficiency virus.

10. The virus-like particles as claimed in claim 8, wherein the polyepitopic sequence is free of methionine codons.

11. The virus-like particles as claimed in claim 8, wherein the polyepitopic sequence of interest is polHIV-1.opt.

12. A composition comprising the virus-like particles as claimed in claim 7 and a pharmaceutically acceptable carrier therefor.

13. A method for optimizing the immunogenicity of a polyepitopic sequence of interest for incorporation in a virus-like particle, wherein the method comprises:
providing a polynucleotide sequence encoding a polyepitopic sequence of interest, wherein the polyepitopic sequence is comprised of epitopes in head-to-tail position;
removing the codons for cysteine and the codons for methionine from the polynucleotide sequence if the epitopes contain cysteine and methionine; and
providing polynucleotides encoding tetra-amino acid spacers between the epitopes in the polyepitopic sequence, wherein each spacer comprises an arginine residue placed in the epitope C₁-position directly linked to a sequence of three different amino acids independently selected from alanine, threonine, lysine, and aspartic acid.

14. The method as claimed in claim 13, which further comprises optimizing codon usage in the polyepitopic sequence based on preferred codon usage patterns in the human genome.

15. A polynucleotide sequence obtained according to the method as claimed in claim 13.

16. An expression vector comprising the polynucleotide sequence as claimed in claim 15.

17. A polyepitopic sequence encoded by the polynucleotide sequence as claimed in claim 15.

18. Virus-like particles comprising the polyepitopic sequence as claimed in claim 17.

19. Virus-like particles as claimed in claim 18, which comprise, as a carrier for the polyepitopic sequence, a VLP chosen from HBsAg, HBc, frCP, HBV/HEV chimeras, yeast Ty, HPV, HCV, and parvovirus.

20. A fusion protein comprising the polyepitopic sequence as claimed in claim 17 positioned within the preS2 region of an M protein of HBV.

21. A polyepitopic amino acid molecule as claimed in claim 17 selected from polHIV-1.opt, pol1A2, pol2A2, pol1B7, and pol2B7.

22. An expression vector for the production of virus-like particles comprising fusion proteins and S proteins of hepatitis B virus (HBV), wherein the proteins are encoded by the preS2+S regions and S region of the HBV genome, respectively, and wherein the expression vector comprises
a polynucleotide sequence that encodes a polypeptide comprising a polyepitopic sequence, wherein epitopes in the polyepitopic sequence are in head to tail position, wherein the polynucleotide sequence is positioned in the preS2 region downstream of the preS2 ATG codon, and wherein the polynucleotide sequence is free of codons for cysteine and contains 0 or 1 codon for methionine apart from a methionine codon necessary to initiate preS2 translation;
polynucleotides encoding tetra-amino acid spacers between the head to tail epitopes in the polyepitopic sequence, wherein each spacer comprises an amino acid residue placed in the epitope C₁-position directly linked to a sequence of three different amino acid residues, wherein the amino acid residues are independently selected from alanine (A), threonine (T), lysine (K), aspartic acid (D), serine (S), glutamine (Q), asparagine (N), and histidine (H);
wherein translation from preS2 and S ATG codons is preserved so that hepatitis B S protein and a fusion protein comprised of M protein and the polypeptide comprising the polyepitopic sequence are expressed, such that the HBsAg proteins and the fusion protein assemble into virus-like particles after expression of the vector in a host cell.

23. The vector as claimed in claim 22, wherein the pathogen is human immunodeficiency virus.

24. The vector as claimed in claim 22, wherein the polyepitopic sequence is free of methionine codons.

25. The vector as claimed in claim 22, wherein the polyepitopic sequence encodes polHIV-1.opt.

26. A host cell comprising the vector as claimed in claim 22.

27. A method of producing virus-like particles, wherein the method comprises:
providing a host cell as claimed in claim 26; and
expressing the fusion protein and the S protein under conditions in which the proteins assemble into virus-like particles, which are released from the host cell into extracellular space.

28. Virus-like particles comprising
fusion protein and HBsAg proteins of hepatitis B virus, wherein the proteins are encoded by preS2+S region and the S region, respectively, of the HBV genome;
a polypeptide fused in-frame in the M protein downstream of the preS2 initiation methionine residue, wherein the polypeptide is free of cysteine residues and contains 0 or 1 methionine residues apart from methionine at the initiation site of preS2 translation, and wherein the polypeptide comprises a polyepitopic sequence of interest, wherein epitopes in the polyepitopic sequence are in head to tail position;
tetra-amino acid spacers between the head to tail epitopes in the polypeptide sequence, wherein each spacer comprises an amino acid residue placed in the epitope C₁-position directly linked to a sequence of three different amino acid residues, wherein the amino acid residues are independently selected from alanine (A), threonine (T), lysine (K), aspartic acid (D), serine (S), glutamine (Q), asparagine (N), and histidine (H);
wherein the HBsAg proteins and the fusion proteins are assembled into the virus-like particles.

29. The virus-like particles as claimed in claim 28, wherein the polypepitopic sequence of interest comes from a human immunodeficiency virus.

30. The virus-like particles as claimed in claim 28, wherein the polyepitopic sequence is free of methionine codons.

31. The virus-like particles as claimed in claim 28, wherein the heterologous polyepitopic sequence is polHIV-1.opt.

32. A composition comprising the virus-like particles as claimed in claim 28 and a pharmaceutically acceptable carrier therefore.

33. A method for optimizing the immunogenicity of a polyepitopic sequence of interest for incorporation in a virus-like particle, wherein the method comprises:
providing a polynucleotide sequence encoding a polyepitopic sequence of interest, wherein the polyepitopic sequence is comprised of epitopes in head-to-tail position;
removing the codons for cysteine and the codons for methionine from the polynucleotide sequence if the epitope contains cysteine and methionine; and
providing polynucleotides encoding tetra-amino acid spacers between the epitopes in the polyepitopic sequence, wherein each spacer comprises an amino acid residue placed in the epitope C₁-position directly linked to a sequence of three different amino acid residues, wherein the amino acid residues are independently selected from alanine (A), threonine (T), lysine (K), aspartic acid (D), serine (S), glutamine (Q), asparagine (N), and histidine (H).

34. The method as claimed in claim 33, which further comprises optimizing codon usage in the polyepitopic sequence based on preferred codon usage patterns in the human genome.

35. A polynucleotide sequence obtained according to the method as claimed in claim 33.

36. An expression vector comprising the polynucleotide sequence as claimed in claim 35.

37. A polyepitopic sequence encoded by the polynucleotide sequence as claimed in claim 35.

38. Virus-like particles comprising the polyepitopic sequence as claimed in claim 37.

39. Virus-like particles as claimed in claim 38, which comprise, as a carrier for the polyepitopic sequence, a VLP chosen from HBsAg, HBc, frCP, HBV/HEV chimeras, yeast Ty, HPV, and parvovirus.

40. A fusion protein comprising the polyepitopic sequence as claimed in claim 37 positioned within the preS2 region of an M protein of HBV.

41. A polyepitopic amino acid molecule selected from polHIV-1.opt, pol1A2, pol2A2, pol1B7, and pol2B7.

42. A bacteria carrying the recombinant vector ppolHIV-1.opt (CNCM I-3547), pGA1xFlagMpol.opt (CNCM I-3544), pGA3xFlagMpol.opt (CNCM I-3546), pGA1xFlagM.pol1A2 (CNCM I-3579), pGA1xFlagM.pol2A2 (CNCM I-3580), pGA1xFlagM.pol1B7 CNCM (I-3581), or pGA1xFlagM.pol2B7 (CNCM I-3582).

43. An expression vector comprising a polynucleotide in a vector in a bacterium as claimed in claim 42, wherein the polynucleotide encodes a recombinant HBSAg virus-like particle.

44. A polyepitope encoded by the polynucleotide inserted in recombinant vectors of claim 42 encoding recombinant HBSAg virus-like particle.


SEQUENCE LISTING (147 sequences)

SEQ ID NO: 1 - protein, 9 aa, Human immunodeficiency virus type 1
SLYNTVATL

SEQ ID NO: 2 - protein, 8 aa, Human immunodeficiency virus type 1
TLNAWVKW

SEQ ID NO: 3 - protein, 10 aa, Human immunodeficiency virus type 1
LLDTGADDTV

SEQ ID NO: 4 - protein, 11 aa, Human immunodeficiency virus type 1
VLDVGDAYFSV

SEQ ID NO: 5 - protein, 9 aa, Human immunodeficiency virus type 1
VIYQYMDDL

SEQ ID NO: 6 - protein, 9 aa, Human immunodeficiency virus type 1
ILKEPVHGV

SEQ ID NO: 7 - protein, 9 aa, Human immunodeficiency virus type 1
PLVKLWYQL

SEQ ID NO: 8 - protein, 11 aa, Human immunodeficiency virus type 1
ESELVNQIIEQ

SEQ ID NO: 9 - protein, 10 aa, Human immunodeficiency virus type 1
ELVNQIIEQL

SEQ ID NO: 10 - protein, 9 aa, Human immunodeficiency virus type 1
LLWKGEGAV

SEQ ID NO: 11 - protein, 9 aa, Human immunodeficiency virus type 1
RLRDLLLIV

SEQ ID NO: 12 - protein, 9 aa, Human immunodeficiency virus type 1
AFHHVAREL

SEQ ID NO: 13 - protein, 6 aa, Artificial Sequence
  Description of Artificial Sequence Synthetic peptide
MQWNST

SEQ ID NO: 14 - DNA, 71 bp, Artificial Sequence
  Description of Artificial Sequence Synthetic primer
gaattcctacttgaaagagccagttcatggggtgagagccaagacctacctgaatgcatgggtgaaagttg

SEQ ID NO: 15 - DNA, 73 bp, Artificial Sequence
  Description of Artificial Sequence Synthetic primer
ctgaatgcatgggtgaaagttgtcagagacaccgcagtgctggatgtgggggatgcctacttctcagtgagag

SEQ ID NO: 16 - DNA, 70 bp, Artificial Sequence
  Description of Artificial Sequence Synthetic primer
atgcctacttctcagtgagagctaagacttatctggtcaaactctggtaccagttgagggctgacactcg

SEQ ID NO: 17 - DNA, 79 bp, Artificial Sequence
  Description of Artificial Sequence Synthetic primer
cagttgagggctgacactcgtctttacaacactgtggccacccttaggaccaaggctcttctggacactggagcagatg

SEQ ID NO: 18 - DNA, 76 bp, Artificial Sequence
  Description of Artificial Sequence Synthetic primer
cttctggacactggagcagatgacactgtgagggctaagaccctgctgtggaagggagagggagcagttaggactg

SEQ ID NO: 19 - DNA, 66 bp, Artificial Sequence
  Description of Artificial Sequence Synthetic primer
aagggagagggagcagttaggactgatgcttacatctaccagtatatggatgaccttagactcgag

SEQ ID NO: 20 - DNA, 34 bp, Artificial Sequence
  Description of Artificial Sequence Synthetic primer
catgaactggctctttcaagtaggaattccactg

SEQ ID NO: 21 - DNA, 35 bp, Artificial Sequence
  Description of Artificial Sequence Synthetic primer
gcagtggaattcctacttgaaagagccagttcatg

SEQ ID NO: 22 - DNA, 34 bp, Artificial Sequence
  Description of Artificial Sequence Synthetic primer
ctatatgctcgagtctaaggtcatccatatactg

SEQ ID NO: 23 - DNA, 312 bp, Artificial Sequence
  Description of Artificial Sequence Synthetic polynucleotide
tacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttaga

SEQ ID NO: 24 - DNA, 27 bp, Artificial Sequence
  Description of Artificial Sequence Synthetic oligonucleotide
tacttgaaagagccagttcatggggtg

SEQ ID NO: 25 - DNA, 27 bp, Artificial Sequence
  Description of Artificial Sequence Synthetic oligonucleotide
tacctgaatgcatgggtgaaagttgtc

SEQ ID NO: 26 - DNA, 33 bp, Artificial Sequence
  Description of Artificial Sequence Synthetic oligonucleotide
gtgctggatgtgggggatgcctacttctcagtg

SEQ ID NO: 27 - DNA, 27 bp, Artificial Sequence
  Description of Artificial Sequence Synthetic oligonucleotide
tatctggtcaaactctggtaccagttg

SEQ ID NO: 28 - DNA, 27 bp, Artificial Sequence
  Description of Artificial Sequence Synthetic oligonucleotide
cgtctttacaacactgtggccaccctt

SEQ ID NO: 29 - DNA, 30 bp, Artificial Sequence
  Description of Artificial Sequence Synthetic oligonucleotide
cttctggacactggagcagatgacactgtg

SEQ ID NO: 30 - DNA, 27 bp, Artificial Sequence
  Description of Artificial Sequence Synthetic oligonucleotide
ctgctgtggaagggagagggagcagtt

SEQ ID NO: 31 - DNA, 27 bp, Artificial Sequence
  Description of Artificial Sequence Synthetic oligonucleotide
tacatctaccagtatatggatgacctt

SEQ ID NO: 32 - protein, 104 aa, Artificial Sequence
  Description of Artificial Sequence Synthetic polypeptide
YLKEPVHGVRAKTYLNAWVKVVRDTAVLDVGDAYFSVRAKTYLVKLWYQLRADTRLYNTVATLRTKALLDTGADDTVRAKTLLWKGEGAVRTDAYIYQYMDDLR

SEQ ID NO: 33 - protein, 9 aa, Human immunodeficiency virus type 1
RLYNTVATL

SEQ ID NO: 34 - protein, 9 aa, Human immunodeficiency virus type 1
YIYQYMDDL

SEQ ID NO: 35 - protein, 9 aa, Human immunodeficiency virus type 1
YLVKLWYQL

SEQ ID NO: 36 - protein, 9 aa, Human immunodeficiency virus type 1
YLKEPVHGV

SEQ ID NO: 37 - protein, 9 aa, Human immunodeficiency virus type 1
YLNAWVKVV

SEQ ID NO: 38 - protein, 9 aa, Artificial Sequence
  Description of Artificial Sequence Synthetic peptide
SLYNTVATL

SEQ ID NO: 39 - protein, 9 aa, Artificial Sequence
  Description of Artificial Sequence Synthetic peptide
YLNAWVKVV

SEQ ID NO: 40 - protein, 10 aa, Artificial Sequence
  Description of Artificial Sequence Synthetic peptide
LLDTGADDTV

SEQ ID NO: 41 - protein, 11 aa, Artificial Sequence
  Description of Artificial Sequence Synthetic peptide
VLDVGDAYFSV

SEQ ID NO: 42 - protein, 9 aa, Artificial Sequence
  Description of Artificial Sequence Synthetic peptide
YIYQYMDDL

SEQ ID NO: 43 - protein, 9 aa, Artificial Sequence
  Description of Artificial Sequence Synthetic peptide
YLKEPVHGV

SEQ ID NO: 44 - protein, 9 aa, Artificial Sequence
  Description of Artificial Sequence Synthetic peptide
YLVKLWYQL

SEQ ID NO: 45 - protein, 9 aa, Artificial Sequence
  Description of Artificial Sequence Synthetic peptide
LLWKGEGAV

SEQ ID NO: 46 - protein, 9 aa, Artificial Sequence
  Description of Artificial Sequence Synthetic peptide
VLAEAMSQV

SEQ ID NO: 47 - protein, 9 aa, Artificial Sequence
  Description of Artificial Sequence Synthetic peptide
SLVKHHMYV

SEQ ID NO: 48 - protein, 9 aa, Artificial Sequence
  Description of Artificial Sequence Synthetic peptide
SPRTLNAWV

SEQ ID NO: 49 - protein, 9 aa, Artificial Sequence
  Description of Artificial Sequence Synthetic peptide
TPQDLNTML

SEQ ID NO: 50 - protein, 9 aa, Artificial Sequence
  Description of Artificial Sequence Synthetic peptide
QPRSDTHVF

SEQ ID NO: 51 - protein, 9 aa, Artificial Sequence
  Description of Artificial Sequence Synthetic peptide
HPVHAGPIA

SEQ ID NO: 52 - protein, 10 aa, Artificial Sequence
  Description of Artificial Sequence Synthetic peptide
RPNNNTRKSI

SEQ ID NO: 53 - protein, 10 aa, Artificial Sequence
  Description of Artificial Sequence Synthetic peptide
APTKAKRRVV

SEQ ID NO: 54 - protein, 9 aa, Artificial Sequence
  Description of Artificial Sequence Synthetic peptide
IPRRIRQGL

SEQ ID NO: 55 - protein, 10 aa, Artificial Sequence
  Description of Artificial Sequence Synthetic peptide
KPVVSTQLLL

SEQ ID NO: 56 - protein, 10 aa, Artificial Sequence
  Description of Artificial Sequence Synthetic peptide
FPVRPQVPLR

SEQ ID NO: 57 - DNA, 192 bp, Artificial Sequence
  Description of Artificial Sequence Synthetic polynucleotide
gtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgaga

SEQ ID NO: 58 - protein, 64 aa, Artificial Sequence
  Description of Artificial Sequence Synthetic peptide
VLDVGDAYFSVRADTYLNAWVKVVRAKTYLVKLWYQLRTDASLVKHHMYVRDTAYIYQYMDDLR

SEQ ID NO: 59 - DNA, 33 bp, Artificial Sequence
  Description of Artificial Sequence Synthetic oligonucleotide
gtgctggatgtgggagatgcctacttctcagtg

SEQ ID NO: 60 - DNA, 27 bp, Artificial Sequence
  Description of Artificial Sequence Synthetic oligonucleotide
tacctgaatgcctgggtgaaggtggtc

SEQ ID NO: 61 - DNA, 27 bp, Artificial Sequence
  Description of Artificial Sequence Synthetic oligonucleotide
tacctggtgaagctgtggtaccagctg

SEQ ID NO: 62 - DNA, 27 bp, Artificial Sequence
  Description of Artificial Sequence Synthetic oligonucleotide
tccctggtgaagcatcacatgtatgtg

SEQ ID NO: 63 - DNA, 27 bp, Artificial Sequence
  Description of Artificial Sequence Synthetic oligonucleotide
tacatctaccagtacatggatgacctg

SEQ ID NO: 64 - DNA, 189 bp, Artificial Sequence
  Description of Artificial Sequence Synthetic polynucleotide
ctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgaga

SEQ ID NO: 65 - protein, 63 aa, Artificial Sequence
  Description of Artificial Sequence Synthetic peptide
LLDTGADDTVRTDASLYNTVATLRADTYLKEPVHGVRAKTLLWKGEGAVRTKAVLAEAMSQVR

SEQ ID NO: 66 - DNA, 30 bp, Artificial Sequence
  Description of Artificial Sequence Synthetic oligonucleotide
ctgcttgacacaggagctgatgacacagtg

SEQ ID NO: 67 - DNA, 27 bp, Artificial Sequence
  Description of Artificial Sequence Synthetic oligonucleotide
agcctgtataacacagtggccaccctg

SEQ ID NO: 68 - DNA, 27 bp, Artificial Sequence
  Description of Artificial Sequence Synthetic oligonucleotide
tacctgaaggagcctgtgcatggagtg

SEQ ID NO: 69 - DNA, 27 bp, Artificial Sequence
  Description of Artificial Sequence Synthetic oligonucleotide
ctcctgtggaagggagagggagcagtg

SEQ ID NO: 70 - DNA, 27 bp, Artificial Sequence
  Description of Artificial Sequence Synthetic oligonucleotide
gtgctggctgaggccatgtcccaggtg

SEQ ID NO: 71 - DNA, 195 bp, Artificial Sequence
  Description of Artificial Sequence Synthetic polynucleotide
tcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcagg

SEQ ID NO: 72 - protein, 65 aa, Artificial Sequence
  Description of Artificial Sequence Synthetic peptide
SPRTLNAWVRAKTRPNNNTRKSIRDTAFPVRPQVPLRRTKAHPVHAGPIARADTAPTKAKRRVVR

SEQ ID NO: 73 - DNA, 27 bp, Artificial Sequence
  Description of Artificial Sequence Synthetic oligonucleotide
tcccctaggaccctgaatgcctgggtg

SEQ ID NO: 74 - DNA, 30 bp, Artificial Sequence
  Description of Artificial Sequence Synthetic oligonucleotide
agacctaacaataacacaaggaagtccatc

SEQ ID NO: 75 - DNA, 30 bp, Artificial Sequence
  Description of Artificial Sequence Synthetic oligonucleotide
ttccctgtgagaccacaggtgcctctgagg

SEQ ID NO: 76 - DNA, 27 bp, Artificial Sequence
  Description of Artificial Sequence Synthetic oligonucleotide
caccctgtgcatgctggccctattgcc

SEQ ID NO: 77 - DNA, 30 bp, Artificial Sequence
  Description of Artificial Sequence Synthetic oligonucleotide
gcacccactaaggccaaaaggagagtggtc

SEQ ID NO: 78 - DNA, 192 bp, Artificial Sequence
  Description of Artificial Sequence Synthetic polynucleotide
aagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgaga

SEQ ID NO: 79 - protein, 64 aa, Artificial Sequence
  Description of Artificial Sequence Synthetic peptide
KPVVSTQLLLRAKTFPVRPQVPLRRADTQPRSDTHVFRTKAIPRRIRQGLRDTATPQDLNTMLR

SEQ ID NO: 80 - DNA, 30 bp, Artificial Sequence
  Description of Artificial Sequence Synthetic oligonucleotide
aagcctgtggtctccacacagctgcttctc

SEQ ID NO: 81 - DNA, 30 bp, Artificial Sequence
  Description of Artificial Sequence Synthetic oligonucleotide
ttccctgtgagaccccaagtgccactgaga

SEQ ID NO: 82 - DNA, 27 bp, Artificial Sequence
  Description of Artificial Sequence Synthetic oligonucleotide
cagcccaggagtgacacccatgtgttc

SEQ ID NO: 83 - DNA, 27 bp, Artificial Sequence
  Description of Artificial Sequence Synthetic oligonucleotide
attcctaggagaattaggcagggcctg

SEQ ID NO: 84 - DNA, 27 bp, Artificial Sequence
  Description of Artificial Sequence Synthetic oligonucleotide
acacctcaggacctgaacaccatgctg

SEQ ID NO: 85 - protein, 4 aa, Artificial Sequence
  Description of Artificial Sequence Synthetic peptide
YMDD

SEQ ID NO: 86 - protein, 9 aa, Influenza A virus
GILGFVFTL

SEQ ID NO: 87 - protein, 16 aa, Hepatitis B virus
QAGFFLLTRILTIPQS

SEQ ID NO: 88 - protein, 15 aa, Hepatitis B virus
TSLNFLGGTTVCLGQ

SEQ ID NO: 89 - protein, 13 aa, Influenza A virus
PKYVKQNTLKLAT

SEQ ID NO: 90 - protein, 121 aa, Artificial Sequence
  Description of Artificial Sequence Synthetic polypeptide
ALVEICTEMYLKEPVHGVYLTPLCVSLYLNAWVKVVYIYQYMDDLYLTFGWCFKLVLDVGDAYFSVYLVKLWYQLSLYNTVATLYMMTACQGVLLDTGADDTVLLWKGEGAVYLTPLCVTL

SEQ ID NO: 91 - protein, 103 aa, Artificial Sequence
  Description of Artificial Sequence Synthetic polypeptide
YLKEPVHGVRAKTYLNAWVKVVRDTAVLDVGDAYFSVRAKTYLVKLWYQLRADTSLYNTVATLRTKALLDTGADDTVRAKTLLWKGEGAVRTDAYIYQYMDDL

SEQ ID NO: 92 - protein, 288 aa, Hepatitis B virus
MGGWSSKPRQGMGTNLSVPNPLGFFPDHQLDPAFGANSNNPDWDFNPNKDHWPDGIKVGAGDFGPGFTPPHGGLLGWSPQAQGILTTVPAAPPPASTNRQSGRQPTPISPPLRDSHPQAMQWNSTTFHQALLDPRVRGLYFPAGGSSSGTVNPVPTTASPISSIFSRTGDPAPNMESTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTS

SEQ ID NO: 93 - protein, 288 aa, Hepatitis B virus
MGGWSSKPRQGMGTNLSVPNPLGFFPDHQLDPAFGANSNNPDWDFNPNKDHWPEAIKVGAGDFGPGFTPPHGGLLGWSPQAQGILTTVPAAPPPVSTNRQSGRQPTPISPPLRDSHPQAMQWNSTTFHQALLDPRVRGLYFPAGGSSSGTVNPVPTTVSPISSIFSRTGDPAPNMESTTSGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYQGMLPVCPLLPGTS

SEQ ID NO: 94 - protein, 277 aa, Hepatitis B virus
MGQNHSVTNPLGFFPDHQLDPLFRANSNNPDWDFNPNKDNWPDATKVGVGAFGPGFTPPHGGLLGWSPQAQGILTTLPAAPPPASTNRQSGRQATPISPPLRDTHPQAMQWNSTVFHQTLQDPRVRGLYFPAGGSSSGTVNPAPTTASHISSIFSRTGDPAPNMDNITSGFLGPLLVLQAGFFLLTKILTIPQSLDSWWTSLNFLGGAPVCPGQNSQSPTSNHSPTSCPPICPGYRWMCLRRFIISLFILLLCLIFLLVLLDYQGMLPVCPLLPGTS

SEQ ID NO: 95 - protein, 277 aa, Hepatitis B virus
MGQNLSTSNPLGFFPEHQLDPAFKANTNNPDWDFNPNKDNWPDANKVGAGAFGPGLTPPHGGLLGWSPQAQGILTTLPINPPPASTNRQSGRQPTPHSPPMRDTHPQAMHWNSTTFHQALQDPRVRGLYFPVGGSSSGTVNPVLNTASHISSIFSRTGDPAPNMETITSGFLGPLLVLQAGFFLLTKILTIPQSLDSWWTSLNFLGGAPVCLGQNSQSPTSNHSPTSCPPICPGYRWMCLRRFIIFLFILLLCLIFLLVLLDYQGMLPVCPLIPGSS

SEQ ID NO: 96 - protein, 278 aa, Hepatitis B virus
MGQQLSWTIPLEWGKNLSTSNPLGFFPEHQLDPAFKANTNNPDWDFNPNKDNWPTANEVGAGAFGPGLTPPHGGLLGWSPQAQGKLITMPANPPPASNKRQSGRQPTPLSPPLRETHPQAMHWNTTTFHQDLAGGSSSGTVSPVPNTVSHISSIFSRTGDPAPNMESITSGFLGPLLVLQAVFFLLTKILTIPQNLDSWWTSLNFLGGAPVCLGQNSQSPTSNHSPTSCPPICPGYRWMCLRRFIIFLFILLLCLIFLLVLLDYQGMLPVCPLIPGSS

SEQ ID NO: 97 - protein, 277 aa, Hepatitis B virus
MGQNLSTSNPLGFFPEHQLDPAFKANTNNPDWDFNPKKDYWPEATKVGAGAFGPGFTPPHGGLLGLSPQAQGILITLPANPPPASTNRQSGRQPTPLSPPLRDTHPQAMQWNSTTFHQALQDPRVRGLYFPAGGSSSGTLNPVPNTASHISSVFSTTGDPAPNMENITSGFLGPLLVLQAGFFLLTKILTIPQSLDSWWTSLNFLGGAPVCLGQNSQSPTSNHSPTSCPPICPGYRWMCLRRFIIFLFILLLCLIFLLVLLDYQGMLPVCPLIPGSS

SEQ ID NO: 98 - protein, 277 aa, Hepatitis B virus
MGQNLSTSNPLGFFPDHQLDPAFRANTANPDWDFNPNKDTWPDANKVGAGAFGLGFTPPHGGLLGWSPQAQGILQTLPANPPPASTNRQSGRQPTPLSPPLRNTHPQAMQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSS

SEQ ID NO: 99 - protein, 277 aa, Hepatitis B virus
MGQNLSTSNPLGFFPDHQLDPAFRANTANPDWDFNPNKDSWPDANKVGAGAFGLGFTPPHGGLLGWSPQAQGILQTLPANPPPASTNRQSGRQPTPLSPPLRNTHPQAMQWNSTTFHQTLQDPRVRGLYLPAGGSSSGTVNPVPTTVSPISSIFSRIGDPALNMENITSGFLGPLLVLQAGFFLLTKILTIPKSLDSWWTSLNFLGGTTVCLGQNSQSPTSNHSPTSCPPTCPGYRWMCLRRFIIFLFILLLCLIFLLVLLDYQGMLPVCPLIPGSS

SEQ ID NO: 100 - protein, 277 aa, Hepatitis B virus
MGQNLSVSNPLGFFPEHQLDPLFRANTNNPDWDFNPNKDTWPEATKVGVGAFGPGFTPPHGGLLGWSPQAQGVTTILPAVPPPASTNRQSGRQPTPISPPLRDTHPQAMQWNSTVFHQTLQDPRVRGLYFPAGGSSSGTVSPVPTSASPISSIFLKTGDPAPNMENISSGFLGPLLVLQAGFFLLTKILTIPQSLDSWWTSLNFLGGAPVCPGQNSQSLTSNHSPTSCPPICPGYRWMCLRRFIIFLFILLLCLIFLLVLLDYQGMLPVCPLLPGTT

SEQ ID NO: 101 - protein, 277 aa, Hepatitis B virus
MGQNLSVTNPLGFFPEHQLDPLFRANTNNPDWDFNPNKDTWPEATKVGVGAFGPGFTPPHGGLLGWSPQAQGVTTILPAVPPPASTNRQSGRRPTPISPPLRDTHPQAMQWNSTVFHQALQDPRVRGLYFPAGGSSSGTVSPVPTTASPISSIFLKTGDPALNMESISSGFLGPLLVLQAGFFLLTKILTIPQSLDSWWTSLNFLGGAPVCPGQNSQSLTSNHSPTSCPPICPGYRWMCLRRFIIFLFILLLCLIFLLVLLDYRGMLPVCPLLPGTT

SEQ ID NO: 102 - protein, 277 aa, Hepatitis B virus
MGLNQSTFNPLGFFPSHQLDPLFKANAGSADWDKNPNKDPWPQAHDTAVGAFGPGLVPPHGGLLGWSSQAQGLSVTVPDTPPPPSTNRDKGRKPTPATPPLRDTHPQAMTWNTSSFQSYLQNPKVRGLYFPAGGSTSSIVNPVPTTASTISSSFSTTGVPVSTMDITSSGFLGPLLALQAVFFLLTKILTMPQSLDSLWTSLNFLGGTPACPGLNSQSPTSSHSPTCCPPTCPGYRWMCLRRSIIFLFILLLCLIFLLVLLDYQGMLPVCPLLPTVT

SEQ ID NO: 103 - protein, 319 aa, Hepatitis B virus
MGNNIKVTFNPDKIAAWWPAVGTYYTTTYPQNQSVFQPGIYQTTSLINPKNQQELDSVLINRYKQIDWNTWQGFPVDQKFSLVSRDPPPKPYINQSAQTFEIKPGPIIVPGIRDIPRGLVPPQTPTNRDQGRKPTPPTPPLRDTHPHLTMKNQTFHLQGFVDGLRDLTTTERQHNAYRDPFITLSPAVPTVSTILSPPSITGDPALSPEMSPSSLLGLLAGLQVVYFLWTKILTIAQNLDWWCTSLSFPGGIPECTGQNSQFQTCKHLPTSCPPTCNGFRWMYLRRFIIYLLVLLLCLIFLLVLLDWKGLIPVCPLQPT

SEQ ID NO: 104 - protein, 319 aa, Hepatitis B virus
MGNNIKVTFNPDKIAAWWPAVGTYYTTTYPQNQSVFQPGIYQTTSLINPKNQQELDSVLINRYKQIDWNTWQGFPVDQKFSLVSRDPPPKPYINQSAQTFEIKPGPIIVPGIRDIPRGLVPPQTPTNRDQGRKPTPPTPPLRDTHPHLTMKNQTFHLQGFVDGLRDLTTTERQHNAYGDPFTTLSPAVPTVSTILSPPSTTGDPALSPEMSPSSLLGLLAGLQVVYFLWTKILTIAQNLDWWWTSLSFPGGIPECTGQNSQFQTCKHLPTSCPPTCNGFRWMYLRRFIIYLLVLLLCLIFLLVLLDWKGFIPVCPLQPT

SEQ ID NO: 105 - protein, 319 aa, Hepatitis B virus
MGNNIKVTFNPDKIAAWWPAVGTYYTTTYPQNQSVFQPGIYQTTSLINPKNQQELDSVLINRYKQIDWNTWQGFPVDQKLPLVSRDPPLKPHINQSAQTFEIKPGPIIVPGIRDIPRGLVPPQTPTNRDQGRKPTPPTPPLRDTHPHLTMKNQTFRLQGFVDGLRDLTTTERYHNAYGDPFITLSPVVPTVSTILSPPSITGDPALSPEMSPSSLLGLLAGLQVVYFLWTKILTIAQNLDWWWTSLSFPGGIPECTGQNSQFQTCKHLPTSCPPTCNGFRWMYLRRFIIYLLVLLLCLIFLLVLLDWKGLIPVCPLQPT

SEQ ID NO: 106 - protein, 316 aa, Hepatitis B virus
MGNNIKVTFDPNKLAAWWPTVGTYYTPTTTVINPAIFKPGIYQTTSLKNPKNQQELDAILMTRYKEIDWDNWQGFPVNQRLPVSNNNPPSGQRAETFEIKSRPIIVPGIRDIPRGIVPPQTPSNRDQRRKPTPLTPPLRDTHPHLTMKNQTGHLQGFAEGLRALTTSDHHNSAYGDPFITLSPVVPTVSTILSPPLTIGDPVLSTEMSPSGLLGLLAGLQVVYFLWTKILTIAQSLDWWWTSLSFPGGIPECTGQNLQFQTCKHLPTSCPPTCNGFRWMYLRRFIIYLLVLLLFLTFLLVLLDWKGLLPVCPMMPA

SEQ ID NO: 107 - protein, 78 aa, Hepatitis B virus
CIPIPSSWAFARFLWEWASVRFSWLSLLVPFVQWFAGLSPTVWLSVIWMMWYWGPSLYNILSPFLPLLPIFFCLWVYI

SEQ ID NO: 108 - protein, 78 aa, Hepatitis B virus; identical to SEQ ID NO: 107

SEQ ID NO: 109 - protein, 78 aa, Hepatitis B virus
CIPIPSSWAFARFLWEWASVRFSWLNLLAPFVQWFAGLSPTVWLLVIWMMWYWGPSLYNILNPFIPLLPIFFCLWVYI

SEQ ID NO: 110 - protein, 78 aa, Hepatitis B virus
CIPIPSSWAFAKFLWEWASVRFSWLSLLAPFVQWFAELSPTVWLSVIWMMWYWGPSLYNILSPFIPLLPIFFCLWVYI

SEQ ID NO: 111 - protein, 78 aa, Hepatitis B virus
CIPIPSSWAFVKFLWEWASVRFSWLSLLAPFVQWFAGLSPTVWLSVIWMMWYWGPSLYNILSPFIPLLPIFFCLWVYI

SEQ ID NO: 112 - protein, 78 aa, Hepatitis B virus
CIPIPSSWAFAKFLWEWASVRFSWLSLLAPFVQWFAGLSPTVWLLAIWMMWYWGPNLYNILSPFIPLLPIFFCLWVYI

SEQ ID NO: 113 - protein, 78 aa, Hepatitis B virus
CIPIPSSWAFGKFLWEWASARFSWLSLLVPFVQWFVGLSPTVWLSVIWMMWYWGPSLYSILSPFLPLLPIFFCLWVYI

SEQ ID NO: 114 - protein, 78 aa, Hepatitis B virus
CIPIPSSWAFGKFLWEWASARFSWLSLLVPFVQWFVGLSPTVWLLVIWMMWYWGPKLFTILSPFLPLLPIFFCLWVYI

SEQ ID NO: 115 - protein, 78 aa, Hepatitis B virus
CIPIPSSWAFARFLWGWASVRFSWLSLLAPFVQWFAGLSPTVWLLVIWMIWYWGPNLFNILSPFIPLLPIFFCLWAYI

SEQ ID NO: 116 - protein, 78 aa, Hepatitis B virus
CIPIPPSWAFAKFLWGWASVRFSWLNLLVPFVQWFAGLSPTVWLSVIWMIWYWGPSLYNILSPFIPLLPIFFCLWAYI

SEQ ID NO: 117 - protein, 78 aa, Hepatitis B virus
CIPIPSSWAFAKFLWDWALARFSWLNSLLPFVQWFAGLSPTVWLLVIWMMWFWGPSLFSILSPFLPLLPLFFWLWAYI

SEQ ID NO: 118 - protein, 78 aa, Hepatitis B virus
CWPIPSSWALGNYLWEWALARLSWLNLLVPLLQWLGGISLIAWFLLIWMIWFWGPALLSILPPFIPIFVLFFLIWVYI

SEQ ID NO: 119 - protein, 78 aa, Hepatitis B virus
CWPIPSSWALGNYLWEWALARFSWLNLLVPLLQWLGGISLIAWFLLIWMIWFWGPALLSILPPFIPIFVLFFLIWVYI

SEQ ID NO: 120 - protein, 78 aa, Hepatitis B virus; identical to SEQ ID NO: 119

SEQ ID NO: 121 - protein, 78 aa, Hepatitis B virus
CWPIPSSWALGSYLWEWALARFSWLSLLVPLLQWLGGISLTVWLLLIWMIWFWGPVLMSILPPFIPIFALFFLIWAYI

SEQ ID NO: 122 - DNA, 313 bp, Artificial Sequence
  Description of Artificial Sequence Synthetic polynucleotide
ctacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttaga

SEQ ID NO: 123 - protein, 105 aa, Artificial Sequence
  Description of Artificial Sequence Synthetic polypeptide
SYLKEPVHGVRAKTYLNAWVKVVRDTAVLDVGDAYFSVRAKTYLVKLWYQLRADTRLYNTVATLRTKALLDTGADDTVRAKTLLWKGEGAVRTDAYIYQYMDDLR

SEQ ID NO: 124 - DNA, 31 bp, Artificial Sequence
  Description of Artificial Sequence Synthetic oligonucleotide
gaattcagctagcgatatccccgggctcgag

SEQ ID NO: 125 - DNA, 5693 bp, Artificial Sequence
  Description of Artificial Sequence Synthetic polynucleotide
aacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaattcagctagcgatatccccgggctcgagcatataggaccagggagagcatttgtggtacctctagaagaagcttgggatccgtcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaata

SEQ ID NO: 126 - DNA, 399 bp, Artificial Sequence
  Description of Artificial Sequence Synthetic polynucleotide
atgcagtggaactccacacccggggctggagcaggagctgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatg

SEQ ID NO: 127 - DNA, 441 bp, Artificial Sequence
  Description of Artificial Sequence Synthetic polynucleotide
atgcagtggaactccacacccggggctggagcaggagctgactacaaagaccacgacggtgattataaagatcacgacattgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatg

SEQ ID NO: 128 - DNA, 5969 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 129 - DNA, 6011 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcgactacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 130 - DNA, 5846 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcgtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 131 - DNA, 5843 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 132 - DNA, 5888 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcgtgctggatgtgggagatgcctacttctcagtgagagctgacacctacctgaatgcctgggtgaaggtggtcagagccaagacctacctggtgaagctgtggtaccagctgaggacagatgcctccctggtgaagcatcacatgtatgtgagagacacagcctacatctaccagtacatggatgacctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 133 - DNA, 5885 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcctgcttgacacaggagctgatgacacagtgaggacagatgccagcctgtataacacagtggccaccctgagagctgacacctacctgaaggagcctgtgcatggagtgagagctaagaccctcctgtggaagggagagggagcagtgagaaccaaggcagtgctggctgaggccatgtcccaggtgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 134 - DNA, 5849 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattctcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcaggctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 135 - DNA, 5846 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgattacaaggacgacgacgacaaggaattcaagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 136 - DNA, 5891 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattctcccctaggaccctgaatgcctgggtgagagctaagaccagacctaacaataacacaaggaagtccatcagagacacagccttccctgtgagaccacaggtgcctctgaggagaaccaaggcccaccctgtgcatgctggccctattgccagagctgatacagcacccactaaggccaaaaggagagtggtcaggctcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 137 - DNA, 5810 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaactccacacccggggctggagcaggagctgactacaaagaccacgacggtgattataaagatcacgacattgattacaaggacgacgacgacaaggaattcaagcctgtggtctccacacagctgcttctcagggccaagaccttccctgtgagaccccaagtgccactgagaagggctgatacacagcccaggagtgacacccatgtgttcagaaccaaggccattcctaggagaattaggcagggcctgagagatacagctacacctcaggacctgaacaccatgctgagactcgagctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcgga

SEQ ID NO: 138 - protein, 104 aa, Artificial Sequence
  Description of Artificial Sequence Synthetic polypeptide
YLNAWVKVVRDTAVLDVGDAYFSVRAKTYLVKLWYQLRADTRLYNTVATLRTKALLDTGADDTVRAKTLLWKGEGAVRTDASYLKEPVHGVRAKTYIYQYMDDL

SEQ ID NO: 139 - protein, 104 aa, Artificial Sequence
  Description of Artificial Sequence Synthetic polypeptide
VLDVGDAYFSVRAKTYLVKLWYQLRADTRLYNTVATLRTKALLDTGADDTVRAKTLLWKGEGAVRTDASYLKEPVHGVRAKTYLNAWVKVVRDTAYIYQYMDDL

SEQ ID NO: 140 - protein, 104 aa, Artificial Sequence
  Description of Artificial Sequence Synthetic polypeptide
YLVKLWYQLRADTRLYNTVATLRTKALLDTGADDTVRAKTLLWKGEGAVRTDASYLKEPVHGVRAKTYLNAWVKVVRDTAVLDVGDAYFSVRAKTYIYQYMDDL

SEQ ID NO: 141 - protein, 104 aa, Artificial Sequence
  Description of Artificial Sequence Synthetic polypeptide
RLYNTVATLRTKALLDTGADDTVRAKTLLWKGEGAVRTDASYLKEPVHGVRAKTYLNAWVKVVRDTAVLDVGDAYFSVRAKTYLVKLWYQLRADTYIYQYMDDL

SEQ ID NO: 142 - protein, 104 aa, Artificial Sequence
  Description of Artificial Sequence Synthetic polypeptide
LLDTGADDTVRAKTLLWKGEGAVRTDASYLKEPVHGVRAKTYLNAWVKVVRDTAVLDVGDAYFSVRAKTYLVKLWYQLRADTRLYNTVATLRTKAYIYQYMDDL

SEQ ID NO: 143 - protein, 104 aa, Artificial Sequence
  Description of Artificial Sequence Synthetic polypeptide
LLWKGEGAVRTDASYLKEPVHGVRAKTYLNAWVKVVRDTAVLDVGDAYFSVRAKTYLVKLWYQLRADTRLYNTVATLRTKALLDTGADDTVRAKTYIYQYMDDL

SEQ ID NO: 144 - protein, 104 aa, Artificial Sequence
  Description of Artificial Sequence Synthetic polypeptide
LLWKGEGAVRTDALLDTGADDTVRAKTRLYNTVATLRTKAYLVKLWYQLRADTVLDVGDAYFSVRAKTYLNAWVKVVRDTASYLKEPVHGVRAKTYIYQYMDDL

SEQ ID NO: 145 - protein, 104 aa, Artificial Sequence
  Description of Artificial Sequence Synthetic polypeptide
LLDTGADDTVRAKTRLYNTVATLRTKAYLVKLWYQLRADTVLDVGDAYFSVRAKTYLNAWVKVVRDTASYLKEPVHGVRAKTLLWKGEGAVRTDAYIYQYMDDL

SEQ ID NO: 146 - protein, 104 aa, Artificial Sequence
  Description of Artificial Sequence Synthetic polypeptide
RLYNTVATLRTKAYLVKLWYQLRADTVLDVGDAYFSVRAKTYLNAWVKVVRDTASYLKEPVHGVLLDTGADDTVRAKTRAKTLLWKGEGAVRTDAYIYQYMDDL

SEQ ID NO: 147 - DNA, 5987 bp, Artificial Sequence
  Description of Artificial Sequence Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaggccatgcagtggaattcctacttgaaagagccagttcatggggtgagagccaagacctacctgaatgcatgggtgaaagttgtcagagacaccgcagtgctggatgtgggggatgcctacttctcagtgagagctaagacttatctggtcaaactctggtaccagttgagggctgacactcgtctttacaacactgtggccacccttaggaccaaggctcttctggacactggagcagatgacactgtgagggctaagaccctgctgtggaagggagagggagcagttaggactgatgcttacatctaccagtatatggatgaccttagactcgagcatataggaccagggagagcatttgtggtacctctagaagaagcttgggatccgtcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtatgcatgtattcaatctaagcaggctttcactttctcgccaacttacaaggcctttctgtgtaaacaatacctgaacctttaccccgttgcccggcaacggccaggtctgtgccaagtgtttgctgacgcaacccccactggctggggcttggtcatgggccatcagcgcatgcgtggaaccttttcggctcctctgccgatccatactgcggaactcctagccgcttgttttgctcgcagcaggtctggagcaaacattatcgggactgataactctgttgtcctatcccgcaaatatacatcgtttccatggctgctaggctgtgctgccaactggatcctgcgcgggacgtcctttgtttacgtcccgtcggcgctgaatcctgcggacgacccttctcggggtcgcttgggactctctcgtccccttctccgtctgccgttccgaccgaccacggggcgcacctctctttacgcggactccccgtctgtgccttctcatctgccggaccgtgtgcacttcgcttcacctctgcacgtcgcatggagaccaccgtgaacgcccaccaaatattgcccaaggtcttacataagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgcgcaccagcaccatgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatccactagttctagagcggccgccaccgcggtggagctccagcttttgttccctttagtgagggttaattgcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc